(19) United States
(12) Patent Application Publication
(10) Pub. No.: US 2024/0124575 A1
(43) Pub. Date: Apr. 18, 2024

(54) HUMAN CD33 ANTIBODY AND USE THEREOF

(71) Applicant: SIMCERE ZAIMING PHARMACEUTICAL CO., LTD., Haikou (CN)

(72) Inventors: Lian XIN, Shanghai (CN); Qiong WANG, Shanghai (CN); Zhuoxiao CAO, Shanghai (CN); Renhong TANG, Shanghai (CN); Jinsheng REN, Nanjing (CN)

(21) Appl. No.: 18/264,486

(22) PCT Filed: Feb. 9, 2022
(86) PCT No.: PCT/CN2022/075621

§ 371 Date: Aug. 7, 2023

(30) Foreign Application Priority Data
Feb. 10, 2021 (CN) 202110182902.1

(51) Int. Cl. C07K 16/28; A61K 47/68; A61K 49/00; A61K 51/10

(52) U.S. Cl. CPC C07K 16/2803; A61K 47/6803; A61K 47/6809; A61K 47/6849; A61K 49/0002; A61K 51/1027; C07K 2317/21; C07K 2317/24; C07K 2317/31; C07K 2317/569

(57) ABSTRACT

An anti-CD33 antibody and a preparation method therefor and an application thereof. The anti-CD33 antibody has high affinity with CD33 protein, and therefore, can be used for preparation of a drug for treating tumor and the like.

The present application claims priority to the Chinese Patent Application No. 202110182902.1 entitled “HUMAN CD33 ANTIBODY AND USE THEREOF”, filed with the China National Intellectual Property Administration on Feb. 10, 2021, which is incorporated herein by reference in its entirety.

### TECHNICAL FIELD

The present invention belongs to the field of bioengineering and biomedicine, and relates to a human CD33 antibody, a nucleic acid for encoding the antibody, a method for preparing the antibody, a pharmaceutical composition comprising the antibody, and related use of the pharmaceutical composition in treating tumors.

### BACKGROUND

In the early 1980s, Andrews et al. identified CD33 as a marker for myeloid leukemia (Blood 62, 24-132, 1983). CD33 is a cell surface antigen specifically expressed on myeloid cells, including myeloid leukemia cells. It is the smallest member of the sialic acid-binding immunoglobulin-like lectin (Siglec) family. CD33 has a molecular weight of 67 kDa and is a type I transmembrane receptor protein consisting of 364 amino acids. The N-terminus of CD33 is positioned outside a cell, and the terminal amino acids constitute a conserved V-set immunoglobulin-like domain and a variable C2-set domain, wherein the V-set specifically recognizes and binds to sialic acid; the cytoplasmic tail has an immunoreceptor tyrosine-based inhibitory motif (ITIM) and an ITIM-like structure, which bind to tyrosine phosphatases to transmit inhibitory signals into cells, so as to achieve the purpose of regulating cell growth. The ITIM sequence in the CD33 molecule differs from other Siglecs in that the hydrophobic amino acids preceding its tyrosine are replaced by leucine and threonine. An analysis of the primary structure of the CD33 molecule in various organisms shows that it is highly conserved.

CD33 is expressed on early multilineage hematopoietic progenitor cells and myelomonocyte precursors. CD33 is not expressed in pluripotent hematopoietic stem cells (Andrews et al., Journal of Experimental Medicine 169, 1721-1731, 1989). The expression of CD33 is down-regulated on mature granulocytes but retained on macrophages, monocytes, and dendritic cells (Andrews et al., Blood 62, 24-132, 1983). In addition to myelomonocytic cells, Valent et al. found that CD33 is expressed on human mast cells and blood basophils (Blood 15, 73(7): 1778-85, 1989).

The extracellular domain of CD33 binding to the sialic acid is involved in cell-to-cell adhesion. Intracellular immunoreceptor tyrosine-based inhibitory motifs (ITIMs) confer cytostatic signals, thereby affecting proliferation and cell survival. The actual signaling pathway for CD33 is still unknown, but von Gunten et al. believed that it involves ITIM and ITIM-like motifs and the recruitment of tyrosine phosphatases (Ann. N.Y. Acad. Sci. 1143: 61-82, 2008). The murine CD33 homologous gene has been defined by Brinkman-Van der Linden et al., but its functional comparability to human CD33 is still questioned (Mol Cell biol., 23(12): 4199-206, 2003). The functional role of human CD33 on normal and malignant leukocytes is still unknown.

Several publications have described that CD33 is a stable cell surface marker expressed on primary AML and CML cells in 70% to 100% of patients tested (Plesa et al., Cancer 112(3), 572-80, 2007; Hauswirt et al., Eur J Clininvest. January 73-82, 2007; Scheinberg et al., Leukemia, Vol. 3, 440-445, 1989). CD33 is expressed on malignant myeloid blast cells (which represent the majority of malignant cells in the peripheral blood and bone marrow of leukemia patients) and on leukemia stem cells (i.e., a relatively small amount of poorly differentiated cells in the bone marrow, which are characterized by their ability to self-renew and maintain the pure line hierarchy of leukemia), while CD33 is expressed only at a low level on some normal hematopoietic stem cells. CD33 can regulate the function of leukocytes during inflammation and immune responses, and thus CD33 is an ideal target for the treatment of acute myeloid leukemia.

A nanobody (Nb) is a genetically engineered antibody comprising only a single domain. In 1993, a Belgian scientist Hamers-Casterman C found a natural heavy chain antibody comprising only heavy chains and no light chains in camel blood (Hamers-Casterman C, Atarhouch T, Muyldermans S, Robinson G, Hamers C, Songa E B, et al. Naturally occurring antibodies devoid of light chains. Nature 363(6428): 446-8 (1993)), wherein the heavy chain antibody, although lacks the light chains, still retains the ability to bind an antigen, compared to a normal antibody. The variable region of the heavy chain antibody in camel body was cloned to obtain a single domain antibody (sdAb) consisting of a heavy chain variable region only; the single domain antibody is called a nanobody or a VHH antibody (variable heavy chain domain of a heavy chain antibody). The molecular weight of a nanobody is only one-tenth of that of a common antibody, moreover, the nanobody has the advantages of more flexible chemical properties, good stability, high solubility, easy expression, high tumor tissue penetrability, and easy coupling with other molecules. Therefore, the nanobody technology has a wide application prospect in the field of therapeutic antibodies.

### SUMMARY

The present invention provides an antibody or an antigen-binding fragment capable of binding to human CD33, a nucleic acid encoding the same, a vector, a cell, a method for preparing the antibody or the antigen-binding fragment, a pharmaceutical composition comprising the antibody or the antigen-binding fragment, and related use of the pharmaceutical composition in treating tumors.

In a first aspect, the present invention provides an antibody or an antigen-binding fragment specifically binding to CD33, comprising: a CDR1, a CDR2, and a CDR3, wherein the CDR1, the CDR2, and the CDR3 have a sequence combination selected from any one of the following sequence combinations or a sequence combination with 1, 2, 3, or more amino acid insertions, deletions, and/or substitutions compared with the sequence combinations;
(1) the CDR1 may be selected from SEQ ID NOs: 29, 32, 35, 38, 41, 44, 47, 50, 53, 56, 59, 62, 65, 68, 71, 74, 77, 80, 83, 86, 89, 92, 95, 98, 101, 104, 107, 110, 113, 116, 119, 122, 125, 128, 131, 134, 137, 140, 143, 146, 149, 152, 155, 158, 161, 164, 167, 170, 173, 176, 179, 182, 185, 188, 226, and 227;
(2) the CDR2 may be selected from SEQ ID NOs: 30, 33, 36, 39, 42, 45, 48, 51, 54, 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150, 153, 156, 159, 162, 165, 168, 171, 174, 177, 180, 183, 186, 189, 229, and 230;
(3) the CDR3 may be selected from SEQ ID NOs: 31, 34, 37, 40, 43, 46, 49, 52, 55, 58, 61, 64, 67, 70, 73, 76, 79, 82, 85, 88, 91, 94, 97, 100, 103, 106, 109, 112, 115, 118, 121, 124, 127, 130, 133, 136, 139, 142, 145, 148, 151, 154, 157, 160, 163, 166, 169, 172, 175, 178, 181, 184, 187, 190, and 228;
each of the CDR1, the CDR2, and the CDR3 is encoded according to a universal analysis method of KABAT, Chothia, or IMGT;
preferably, the substitutions are conservative amino acid substitutions.

In some embodiments, the CDR1, the CDR2, and the CDR3 comprise a CDR1, a CDR2, and a CDR3 in a VHH domain set forth in any one of SEQ ID NOs: 11 to 28 and 193 to 223, respectively;
preferably, according to the KABAT, Chothia, or IMGT numbering scheme, the antibody or the antigen-binding fragment comprises a CDR1, a CDR2, and a CDR3:
(1) the CDR1 is selected from SEQ ID NOs: 29, 83, and 137, the CDR2 is selected from SEQ ID NOs: 30, 84, and 138, and the CDR3 is selected from SEQ ID NOs: 31, 85, and 139;
(2) the CDR1 is selected from SEQ ID NOs: 32, 86, and 140, the CDR2 is selected from SEQ ID NOs: 33, 87, and 141, and the CDR3 is selected from SEQ ID NOs: 34, 88, and 142;
(3) the CDR1 is selected from SEQ ID NOs: 35, 89, 143, 226, and 227, the CDR2 is selected from SEQ ID NOs: 36, 90, and 144, and the CDR3 is selected from SEQ ID NOs: 37, 91, and 145;
(4) the CDR1 is selected from SEQ ID NOs: 38, 92, and 146, the CDR2 is selected from SEQ ID NOs: 39, 93, and 147, and the CDR3 is selected from SEQ ID NOs: 40, 94, and 148;
(5) the CDR1 is selected from SEQ ID NOs: 41, 95, and 149, the CDR2 is selected from SEQ ID NOs: 42, 96, 150, and 229, and the CDR3 is selected from SEQ ID NOs: 43, 97, 151, and 228;
(6) the CDR1 is selected from SEQ ID NOs: 44, 98, and 152, the CDR2 is selected from SEQ ID NOs: 45, 99, and 153, and the CDR3 is selected from SEQ ID NOs: 46, 100, and 154;
(7) the CDR1 is selected from SEQ ID NOs: 47, 101, and 155, the CDR2 is selected from SEQ ID NOs: 48, 102, and 156, and the CDR3 is selected from SEQ ID NOs: 49, 103, and 157;
(8) the CDR1 is selected from SEQ ID NOs: 50, 104, and 158, the CDR2 is selected from SEQ ID NOs: 51, 105, and 159, and the CDR3 is selected from SEQ ID NOs: 52, 106, and 160:
(9) the CDR1 is selected from SEQ ID NOs: 53, 107, and 161, the CDR2 is selected from SEQ ID NOs: 54, 108, 162, and 230, and the CDR3 is selected from SEQ ID NOs: 55, 109, and 163;
(10) the CDR1 is selected from SEQ ID NOs: 56, 110, and 164, the CDR2 is selected from SEQ ID NOs: 57, 111, and 165, and the CDR3 is selected from SEQ ID NOs: 58, 112, and 166;
(11) the CDR1 is selected from SEQ ID NOs: 59, 113, and 167, the CDR2 is selected from SEQ ID NOs: 60, 114, and 168, and the CDR3 is selected from SEQ ID NOs: 61, 115, and 169:
(12) the CDR1 is selected from SEQ ID NOs: 62, 116, and 170, the CDR2 is selected from SEQ ID NOs: 63, 117, and 171, and the CDR3 is selected from SEQ ID NOs: 64, 118, and 172;
(13) the CDR1 is selected from SEQ ID NOs: 65, 119, and 173, the CDR2 is selected from SEQ ID NOs: 66, 120, and 174, and the CDR3 is selected from SEQ ID NOs: 67, 121, and 175;
(14) the CDR1 is selected from SEQ ID NOs: 68, 122, and 176, the CDR2 is selected from SEQ ID NOs: 69, 123, and 177, and the CDR3 is selected from SEQ ID NOs: 70, 124, and 178;
(15) the CDR1 is selected from SEQ ID NOs: 71, 125, and 179, the CDR2 is selected from SEQ ID NOs: 72, 126, and 180, and the CDR3 is selected from SEQ ID NOs: 73, 127, and 181;
(16) the CDR1 is selected from SEQ ID NOs: 74, 128, and 182, the CDR2 is selected from SEQ ID NOs: 75, 129, and 183, and the CDR3 is selected from SEQ ID NOs: 76, 130, and 184,
(17) the CDR1 is selected from SEQ ID NOs: 77, 131, and 185, the CDR2 is selected from SEQ ID NOs: 78, 132, and 186, and the CDR3 is selected from SEQ ID NOs: 79, 133, and 187;
(18) the CDR1 is selected from SEQ ID NOs: 80, 134, and 188, the CDR2 is selected from SEQ ID NOs: 81, 135, and 189, and the CDR3 is selected from SEQ ID NOs: 82, 136, and 190; or
(19) the CDR1, the CDR2, and the CDR3 have a sequence combination with 1, 2, 3, or more amino acid insertions, deletions, and/or substitutions compared with the sequence combinations (1) to (18) described above; and preferably, the substitutions are conservative amino acid substitutions.

In some embodiments, preferably, the antibody or the antigen-binding fragment comprises a sequence combination of CDR1, CDR2, and CDR3 selected from SEQ ID NOs: 11 to 28 and 193 to 223, or comprises sequences having at least 80%, 85%, 90%, 91%, 92%, 93%, 94%, 95%, 96%, 97%, 98%, 99%, or 100% identity to the CDR1, the CDR2, and/or the CDR3 described above.

In some embodiments, preferably, the antibody or the antigen-binding fragment comprises an FR region in a VHH domain set forth in any one of SEQ ID NOs: 11 to 28 and 193 to 223; optionally, the antibody or the antigen-binding fragment comprises a sequence having at least 80%, 85%, 90%, 91%, 92%, 93%, 94%, 95%, 96%, 97%, 98%, 99%, or 100% identity to the FR region in the VHH domain set forth in any one of SEQ ID NOs: 11 to 28 and 193 to 223; or, optionally, the antibody or the antigen-binding fragment comprises a sequence having at most 15, 14, 13, 12, 11, 10, 9, 8, 7, 6, 5, 4, 3, 2, or 1 mutation compared with the FR region in the VHH domain set forth in any one of SEQ ID NOs: 11 to 28 and 193 to 223; the mutation may be selected from an insertion, a deletion, and/or a substitution; the substitution is preferably a conservative amino acid substitution.

In some embodiments, preferably, the antibody or the antigen-binding fragment comprises a sequence set forth in any one of SEQ ID NOs: 11 to 28 and 193 to 223; optionally, the antibody or the antigen-binding fragment comprises a sequence having at least 80%, 85%, 90%, 91%, 92%, 93%, 94%, 95%, 96%, 97%, 98%, 99%, or 100/c identity to the sequence set forth in any one of SEQ ID NOs: 11 to 28 and 193 to 223; or, optionally, the antibody or the antigen-binding fragment comprises a sequence having at most 20, 19, 18, 17, 16, 15, 14, 13, 12, 11, 10, 9, 8, 7, 6, 5, 4, 3, 2, or 1 mutation compared with the sequence set forth in any one of SEQ ID NOs: 11 to 28 and 193 to 223; the mutation may be selected from an insertion, a deletion, and/or a substitution; the substitution is preferably a conservative amino acid substitution.

In some embodiments, preferably, the antibody or the antigen-binding fragment binds to human CD33 with a dissociation constant (KD) of not greater than 100 nM, and binds to monkey CD33 with a KD of not greater than 100 nM.

Further, in some embodiments, the antibody or the antigen-binding fragment comprises or does not comprise an antibody heavy chain constant region; optionally, the antibody heavy chain constant region may be selected from human, Vicugna pacos, mouse, rat, rabbit, and sheep; optionally, the antibody heavy chain constant region may be selected from IgG, IgM, IgA, IgE, and IgD, and the IgG may be selected from IgG1, IgG2, IgG3, and IgG4; optionally, the heavy chain constant region may be selected from an Fc region, a CH3 region, a heavy chain constant region without a CH1 fragment, and an intact heavy chain constant region; preferably, the heavy chain constant region is a human Fc region, more preferably having an amino acid sequence set forth in SEQ ID NO: 191; preferably, the antibody or the antigen-binding fragment is a heavy chain antibody.

Further, in some embodiments, the antibody or the antigen-binding fragment is: (1) a chimeric antibody or a fragment thereof; (2) a humanized antibody or a fragment thereof; or (3) a full human antibody or a fragment thereof.

Further, in some embodiments, the antibody or the antigen-binding fragment is further conjugated to a therapeutic agent or a tracer; preferably, the therapeutic agent is selected from a radioisotope, a cytotoxic agent, and an immunomodulator, and the tracer is selected from a radiocontrast medium, a paramagnetic ion, a metal, a fluorescent label, a chemiluminescent label, an ultrasound contrast agent, and a photosensitizer; more preferably, the cytotoxic agent is selected from the group consisting of alkaloids, methotrexate, doxorubicin, and taxanes.

Further, in some embodiments, the antibody or the antigen-binding fragment is further conjugated to another functional molecule, wherein the functional molecule may be selected from one or more of a signal peptide, a protein tag, and a cytokine.

In a second aspect, the present invention provides a multispecific antibody, wherein the multispecific antibody comprises the antibody or the antigen-binding fragment according to the first aspect; preferably, the multispecific antibody further comprises an antibody or an antigen-binding fragment specifically binding to an antigen other than CD33 or binding to an epitope of CD33 different from that of the antibody or the antigen-binding fragment according to the first aspect.

In some embodiments, preferably, the antigen other than CD33 may be selected from: CD3, preferably CD3ε; CD16, preferably CD16A; CS32B; PD-1; PD-2; PD-L1; NKG2D; CD19; CD20; CD40; CD47; 4-1BB; CD137; EGFR; EGFRvIII; TNF-alpha; MSLN; HER2; HER3; HSA; CD5; CD27; EphA2; EpCAM; MUC1; MUC16; CEA; Claudin18.2; folate receptor; Claudin6; WT1; NY-ESO-1; MAGE3; ASGPR1; and CDH16.

In some embodiments, preferably, the multispecific antibody may be a bispecific antibody, a trispecific antibody, or a tetraspecific antibody, and may be bivalent, tetravalent, or hexavalent.

In a third aspect, the present invention provides a chimeric antigen receptor (CAR), at least comprising an extracellular antigen-binding domain, a transmembrane domain, and an intracellular signaling domain, wherein the extracellular antigen-binding domain comprises the antibody or the antigen-binding fragment according to the first aspect.

In a fourth aspect, the present invention provides an immune effector cell, wherein the immune effector cell expresses the chimeric antigen receptor according to the third aspect or comprises a nucleic acid fragment encoding the chimeric antigen receptor according to the third aspect; preferably, the immune effector cell is selected from a T cell, a natural killer (NK) cell, a natural killer T (NKT) cell, a double negative T (DNT) cell, a monocyte, a macrophage, a dendritic cell, and a mast cell; the T cell is preferably selected from a cytotoxic T cell, a regulatory T cell, and a helper T cell; preferably, the immune effector cell is an autoimmune effector cell or an allogeneic immune effector cell.

In a fifth aspect, the present invention provides an isolated nucleic acid fragment capable of encoding the antibody or the antigen-binding fragment according to the first aspect, the multispecific antibody according to the second aspect, or the chimeric antigen receptor according to the third aspect described above.

In a sixth aspect, the present invention provides a vector comprising the isolated nucleic acid fragment according to the fifth aspect.

In a seventh aspect, the present invention provides a host cell, wherein the host cell comprises the vector according to the sixth aspect described above; preferably, the cell is a prokaryotic cell or a eukaryotic cell, e.g., a bacterial (E. coli) cell, a fungal (yeast) cell, an insect cell, or a mammalian cell (CHO cell line or 293T cell line).

In an eighth aspect, the present invention further provides a method for preparing an antibody or an antigen-binding fragment or a multispecific antibody, wherein the method comprises: culturing the cell according to the seventh aspect described above, and isolating an antibody or an antigen-binding fragment expressed by the cell or a multispecific antibody expressed by the cell in a suitable condition.

In a ninth aspect, the present invention further provides a method for preparing an immune effector cell, wherein the method comprises introducing a nucleic acid fragment encoding the CAR according to the third aspect into the immune effector cell; optionally, the method further comprises initiating expression of the CAR according to the third aspect in the immune effector cell.

In a tenth aspect, the present invention further provides a pharmaceutical composition, wherein the pharmaceutical composition comprises the antibody or the antigen-binding fragment according to the first aspect, the multispecific antibody according to the second aspect, the immune effector cell according to the fourth aspect, the nucleic acid fragment according to the fifth aspect, the vector according to the sixth aspect, or a product prepared by the method according to the eighth or ninth aspect; optionally, the pharmaceutical composition further comprises a pharmaceutically acceptable carrier, diluent, or adjuvant; optionally, the pharmaceutical composition further comprises an additional antineoplastic agent.

In an eleventh aspect, the present invention further provides a method for preventing and/or treating a tumor, comprising: administering to a patient in need thereof an effective amount of the antibody or the antigen-binding fragment according to the first aspect, the multispecific antibody according to the second aspect, the immune effector cell according to the fourth aspect, the nucleic acid fragment according to the fifth aspect, the vector according to the sixth aspect, a product prepared by the method according to the eighth or ninth aspect, or the pharmaceutical composition according to the tenth aspect, wherein the tumor is preferably selected from myelodysplastic syndrome (MDS), acute myelogenous leukemia (AML), chronic myelogenous leukemia (CML), and promyelocytic leukemia (PML).

In a twelfth aspect, the present invention provides use of the antibody or the antigen-binding fragment according to the first aspect, the multispecific antibody according to the second aspect, the immune effector cell according to the fourth aspect, the nucleic acid fragment according to the fifth aspect, the vector according to the sixth aspect, a product prepared by the method according to the eighth or ninth aspect, or the pharmaceutical composition according to the tenth aspect in the manufacture of a medicament for preventing and/or treating a tumor, wherein the tumor is preferably selected from myelodysplastic syndrome (MDS), acute myelogenous leukemia (AML), chronic myelogenous leukemia (CML), and promyelocytic leukemia (PML).

In a thirteenth aspect, the present invention provides a kit, comprising the antibody or the antigen-binding fragment according to the first aspect, the multispecific antibody according to the second aspect, the immune effector cell according to the fourth aspect, the nucleic acid fragment according to the fifth aspect, the vector according to the sixth aspect, or a product prepared by the method according to the eighth or ninth aspect.

In a fourteenth aspect, the present invention provides a method for inhibiting the proliferation or migration of a cell expressing CD33 in vitro, comprising: contacting the cell with the antibody or the antigen-binding fragment according to the first aspect in a condition allowing formation of a complex by the antibody or the antigen-binding fragment according to the first aspect and CD33.

### Terms and Definitions

Unless otherwise stated, the terms used in the present invention have the meanings that are commonly understood by those of ordinary skill in the art. For a term explicitly defined in the present invention, the meaning of the term shall be subject to the stated definition.

Furthermore, unless otherwise stated herein, terms used in the singular form herein shall include the plural form, and vice versa. More specifically, as used in this specification and the appended claims, unless otherwise clearly indicated, the singular forms “a”, “an”, and “the” include referents in the plural form.

The terms “including”, “comprising”, and “having” herein are used interchangeably and are intended to indicate the inclusion of a solution, implying that there may be elements other than those listed in the solution. Meanwhile, it should be understood that the descriptions “including”, “comprising”, and “having” as used herein also provide the solution of “consisting of . . . ”.

The term “and/or” as used herein includes the meanings of “and”, “or”, and “all or any other combination of elements linked by the term”.

The term “optional” or “optionally” herein means that the event or circumstance subsequently described may, but does not necessarily, occur, and that the description includes instances where the event or circumstance occurs or does not occur. For example, “optionally comprising 1 to 3 antibody heavy chain variable regions” means that the antibody heavy chain variable regions may, but do not have to, be present, and if present, in an amount of 1, 2, or 3.

The term “CD33” herein means the smallest member of the sialic acid-binding immunoglobulin-like lectin (Siglec) family. CD33 has a molecular weight of 67 kDa and is a type I transmembrane receptor protein consisting of 364 amino acids. The N-terminus of CD33 is positioned outside a cell, and the terminal amino acids constitute a conserved V-set immunoglobulin-like domain and a variable C2-set domain, wherein the V-set specifically recognizes and binds to sialic acid; the cytoplasmic tail has an immunoreceptor tyrosine-based inhibitory motif (ITIM) and an ITIM-like structure, which bind with tyrosine phosphatases to transmit inhibitory signals into cells, so as to achieve the purpose of regulating cell growth. The ITIM sequence in the CD33 molecule differs from other Siglecs in that the hydrophobic amino acids preceding its tyrosine are replaced by leucine and threonine. An analysis of the primary structure of the CD33 molecule in various organisms shows that it is highly conserved. The term “CD33” includes CD33 proteins of any human and non-human animal species, and specifically includes human CD33 as well as CD33 of non-human mammals.

The term “specific binding” herein means that an antigen-binding molecule (e.g., an antibody) specifically binds to an antigen and substantially identical antigens, generally with high affinity, but does not bind to unrelated antigens with high affinity. Affinity is generally reflected in an equilibrium dissociation constant (KD), where a low KD indicates a high affinity. In the case of antibodies, a high affinity generally means having a KD of about 10⁻⁷ M or less, about 10⁻⁸ M or less, about 1×10⁻⁹ M or less, about 1×10⁻¹⁰ M or less, 1×10⁻¹¹ M or less, or 1×10⁻¹² M or less. KD is calculated as follows: KD=Kd/Ka, where Kd represents the dissociation rate and Ka represents the association rate. The equilibrium dissociation constant KD may be measured using a method well known in the art, such as surface plasmon resonance (e.g., Biacore) or equilibrium dialysis.

The term “antigen-binding molecule” herein is used in its broadest sense and refers to a molecule that specifically binds to an antigen. Exemplarily, antigen-binding molecules include, but are not limited to, antibodies or antibody mimetics. “Antibody mimetic” refers to an organic compound or a binding domain that is capable of specifically binding to an antigen, but is not structurally related to an antibody. Exemplarily, the antibody mimetic includes, but is not limited to, affibody, affitin, affilin, a designed ankyrin repeat protein (DARPin), a nucleic acid aptamer, and a Kunitz domain peptide.

The term “antibody” herein is used in its broadest sense and refers to a polypeptide or a combination of polypeptides that comprises sufficient sequence from an immunoglobulin heavy chain variable region and/or sufficient sequence from an immunoglobulin light chain variable region to be capable of specifically binding to an antigen. “Antibody” herein encompasses various forms and various structures as long as they exhibit the desired antigen-binding activity. “Antibody” herein includes alternative protein scaffolds or artificial scaffolds having grafted complementarity determining regions (CDRs) or CDR derivatives. Such scaffolds include antibody-derived scaffolds comprising mutations introduced to, e.g., stabilize the three-dimensional structure of the antibody, and fully synthetic scaffolds comprising, e.g., biocompatible polymers. See, e.g., Korndorfer et al., 2003, Proteins: Structure, Function, and Bioinformatics, 53(1): 121-129 (2003); and Roque el al., Biotechnol. Prog. 20: 639-654 (2004). Such scaffolds may also include non-antibody derived scaffolds, such as scaffold proteins known in the art to be useful for grafting CDRs, including, but not limited to tenascin, fibronectin, peptide aptamers, and the like.

The term “antibody” herein includes a typical “four-chain antibody”, which is an immunoglobulin consisting of two heavy chains (HCs) and two light chains (LCs). The heavy chain refers to a polypeptide chain consisting of, from the N-terminus to the C-terminus, a heavy chain variable region (VH), a heavy chain constant region CH1 domain, a hinge region (HR), a heavy chain constant region CH2 domain, a heavy chain constant region CH3 domain; moreover, when the full-length antibody is of IgE isoform, the heavy chain optionally further comprises a heavy chain constant region CH4 domain. The light chain is a polypeptide chain consisting of, from the N-terminus to the C-terminus, a light chain variable region (VL) and a light chain constant region (CL). The heavy chains are connected to each other and to the light chains through disulfide bonds to form a Y-shaped structure. The heavy chain constant regions of immunoglobulins differ in their amino acid composition and arrangement, and thus in their antigenicity. Accordingly, “immunoglobulin” herein may be divided into five classes, or isotypes of immunoglobulins, i.e., IgM, IgD, IgG, IgA, and IgE, with their corresponding heavy chains being μ, δ, γ, α, and ε chains, respectively. The Ig of the same class may be divided into different subclasses according to the differences in the amino acid composition of the hinge regions and the number and location of disulfide bonds in the heavy chains. For example, IgG may be divided into IgG1, IgG2, IgG3, and IgG4; and IgA may be divided into IgA1 and IgA2. Light chains are divided into κ or λ chains according to differences in the constant regions. Each of the five classes of Ig may have a κ chain or a λ chain.

The term “antibody” herein also includes antibodies exclusive of a light chain, e.g., heavy chain antibodies (HCAbs) produced by Camelus dromedarius, Camelus baciriamis, Lama glama, Lama guanicoe, Vicugna pacos, and the like, as well as immunoglobulin new antigen receptors (IgNARs) found in Chondrichthyes, e.g., shark.

The term “heavy chain antibody” herein refers to a second type of antibody known to occur naturally in species in the family Camelidae, and the heavy chain antibody naturally lacks a light chain and a CH1 constant region. The heavy chain antibody (HCAb) consists of two heavy chains which are covalently linked by disulfide bonds. One end of each heavy chain in the HCAb has a variable domain. In order to distinguish it from the variable domains (VHs) of heavy chains of a “conventional” camelid antibody, the variable domains of the HCAb are referred to as “VHHs”. The VHH domains are completely different from the VH domains, and they are encoded by different gene segments in the camelid genome.

The terms “VHH domain”, “nanobody”, and “single domain antibody (sdAb)” herein have the same meaning and can be used interchangeably, and refer to a single domain antibody consisting of only one heavy chain variable region constructed by cloning a variable region of a heavy chain antibody, which is the smallest antigen-binding fragment having the complete function. Generally, a single domain antibody consisting of only one heavy chain variable region is constructed by obtaining a heavy chain antibody naturally lacking a light chain and a heavy chain constant region 1 (CH1) and then cloning a variable region of an antibody heavy chain.

For further description of “heavy chain antibody”, “single domain antibody”, “VHH domain” and “nanobody”, see. Hamers-Casterman et al., Nature. 1993; 363; 446-8; a review article (Reviews in Molecular Biotechnology 74: 277-302, 2001) by Muyldermans; and the following patent applications mentioned as general background art: WO 94/04678, WO 95/04079, and WO 96/34103; WO94/25591, WO 99/37681, WO 00/40968, WO 00/43507, WO 00/65057, WO 01/40310, WO 01/44301, EP 1134231, and WO 02/48193; WO97/49805, WO 01/21817, WO 03/035694, WO 03/054016, and WO 03/055527; WO 03/050531; WO 01/90190, WO03/025020; and WO 04/041867, WO 04/041862, WO 04/041865, WO 04/041863, WO 04/062551, WO 05/044858, WO 06/40153, WO 06/079372, WO 06/122786, WO 06/122787, and WO 06/122825 as well as other prior art mentioned in these applications.

“Antibody” herein may be derived from any animal, including, but not limited to, human and non-human animals which may be selected from primates, mammals, rodents, and vertebrates, such as Camelidae species, Lama glama, Lama guanicoe, Vicugna pacos, sheep, rabbits, mice, rats, or Chondrichthves (e.g., shark). As used herein, the term “antigen-binding fragment” refers to one or more fragments of an antibody that retain the ability to specifically bind to a target antigen. The antigen-binding function of an antibody may be performed by fragments of a full-length antibody. An antibody fragment may be an Fab, F(ab′)₂, scFv, SMIP, diabody, triabody, affibody, nanobody, aptamer, or domain antibody. Examples of binding fragments encompassing the term “antigen-binding fragment” of an antibody include, but are not limited to: (i) an Fab fragment, i.e., a monovalent fragment consisting of VL, VH, CL, and CH1 domains; (ii) an F(ab)₂ fragment, i.e., a bivalent fragment comprising two Fab fragments linked by a disulfide bond at the hinge region; (iii) an Fd fragment consisting of VH and CH1 domains; (iv) an Fv fragment consisting of VL and VH domains of a single arm of an antibody; (v) a dAb comprising VH and VL domains; (vi) a dAb fragment consisting of a VH domain (Ward et al., Nature 341: 544-546, 1989); (vii) a dAb consisting of a VH or VL domain; (viii) an isolated complementarity determining region (CDR); (ix) a heavy chain antibody fragment consisting of VHH, CH2, and CH3; and (x) a combination of two or more isolated CDRs, which may optionally be joined by a synthetic linker. Furthermore, although the two domains (VL and VH) of the Fv fragment are encoded by separate genes, these two domains may be joined using a recombination method through a linker that enables them to be made into a single protein chain in which the VL and VH regions are paired to form a monovalent molecule (known as single chain Fv (scFv); see, e.g., Bird et al., Science 242: 423-426, 1988 and Huston et al., Proc. Natl. Acad. Sci. USA 85: 5879-5883, 1988). These antibody fragments may be obtained using a conventional technique known to those skilled in the art, and these fragments are screened for use in the same manner as intact antibodies. Antigen-binding fragments may be produced by a recombinant DNA technique or enzymatic or chemical cleavage of intact immunoglobulins or, in some embodiments, by a chemical peptide synthesis procedure known in the art.

The term “monoclonal antibody” herein refers to an antibody derived from a single clone (including any eukaryotic, prokaryotic, or phage clone), and is not limited to the production method of the antibody.

The term “multispecific” herein means having at least two antigen-binding sites, each of which binds to a different epitope of the same antigen or a different epitope of a different antigen. Thus, the terms such as “bispecific”, “trispecific”, and “tetraspecific” refer to the number of different epitopes to which an antibody/antigen-binding molecule can bind.

The term “valent” herein refers to the presence of a specified number of binding sites in an antibody/antigen-binding molecule. Thus, the terms “monovalent”, “divalent”, “tetravalent”, and “hexavalent” refer to the presence of one binding site, two binding sites, four binding sites, and six binding sites, respectively, in an antibody/antigen-binding molecule.

“Full-length antibody”, “complete antibody”, and “intact antibody” herein are used interchangeably and refer to an antibody having a structure substantially similar to that of a natural antibody.

“Antigen-binding fragment” and “antibody fragment” herein are used interchangeably and do not have the entire structure of an intact antibody, but comprise only a portion of the intact antibody or a variant of the portion that has the ability to bind to an antigen. Exemplarily, the “antigen-binding fragment” and “antibody fragment” may be single domain antibodies.

The term “chimeric antibody” herein refers to an antibody having a variable sequence of an immunoglobulin derived from a microbial source (e.g., rat, mouse, rabbit, or Vicugna pacos) and a constant region of an immunoglobulin derived from a different organism (e.g., human). Methods for producing the chimeric antibody are known in the art. See, e.g., Morrison, 1985, Science 229(4719): 1202-7; Oi et al., 1986, Bio Techniques 4: 214-221; Gillies et al., 1985 J Immunol Methods 125: 191-202, which are incorporated herein by reference.

The term “humanized antibody” herein refers to a genetically engineered non-human antibody that has an amino acid sequence modified to increase homology to the sequence of a human antibody. Generally, all or part of the CDR regions of a humanized antibody are derived from a non-human antibody (donor antibody), and all or part of the non-CDR regions (e.g., variable region FRs and/or constant regions) are derived from a humanized immunoglobulin (receptor antibody). The humanized antibody generally retains or partially retains the desired properties of the donor antibody, including, but not limited to, antigen specificity, affinity, reactivity, the ability to increase the activity of immune cells, the ability to enhance immune response, and the like.

The term “fully human antibody” herein refers to an antibody having variable regions in which both the FRs and CDRs are derived from human germline immunoglobulin sequences. Furthermore, if the antibody comprises constant regions, the constant regions are also derived from human germline immunoglobulin sequences. The fully human antibody herein may include amino acid residues not encoded by human germline immunoglobulin sequences (e.g., mutations introduced by random or site-specific mutagenesis in vitro or by somatic mutations in vivo). However, “fully human antibody” herein does not include antibodies in which CDR sequences derived from the germline of another mammalian species (e.g., mouse) have been grafted onto human framework sequences.

The term “variable region” herein refers to a region of a heavy or light chain of an antibody involved in the binding of the antibody to an antigen. “Heavy chain variable region” is used interchangeably with “VH” and “HCVR”, and “light chain variable region” is used interchangeably with “VL” and “LCVR”. Heavy and light chain variable domains (VH and VL, respectively) of natural antibodies generally have similar structures, each of which contains four conserved framework regions (FRs) and three hypervariable regions (HVRs). See, e.g., Kindt et al., Kuby Immunology, 6th ed., W. H. Freeman and Co., p. 91 (2007). A single VH or VL domain may be sufficient to provide antigen-binding specificity. The terms “complementarity determining region” and “CDR” herein are used interchangeably and generally refer to a hypervariable region (HVR) of a heavy chain variable region (VH) or a light chain variable region (VL), which is also known as the complementarity determining region because it is precisely complementary to an epitope in a spatial structure, wherein the heavy chain variable chain CDR may be abbreviated as HCDR and the light chain variable chain CDR may be abbreviated as LCDR. The terms “framework region” or “FR region” are used interchangeably and refer to those amino acid residues of an antibody heavy chain variable region or light chain variable region, other than the CDRs. In general, a typical antibody variable region consists of 4 FR regions and 3 CDR regions in the following order. FR1-CDR1-FR2-CDR2-FR3-CDR3-FR4 (see Kabat et al., Sequences of Proteins of Immunological Interest, National Institute of Health, Bethesda, Md. 1987; which is incorporated herein by reference). For example, herein, CDR1-VH, CDR2-VH, and CDR3-VH refer to the first CDR, second CDR, and third CDR, respectively, of the heavy chain variable region (VH), which constitute a CDR combination (VHCDR combination) of the heavy chain (or the variable region thereof); CDR1-VL, CDR2-VL, and CDR3-VL refer to the first CDR, second CDR, and third CDR, respectively, of the light chain variable region (VL), which constitute a CDR combination (VLCDR combination) of the light chain (or the variable region thereof).

For further description of the CDRs, see Kabat et al., J. Biol. Chem., 252: 6609-6616 (1977); Kabat et al., United States Department of Health and Human Services, “Sequences of proteins of immunological interest” (1991); Chothia et al., J. Mol. Biol. 196: 901-917 (1987); Al-Lazikani B. et al., J. Mol. Biol., 273: 927-948 (1997); MacCallum et al., J. Mol. Biol. 262: 732-745 (1996); Abhinandan and Martin, Mol. Immunol., 45: 3832-3839 (2008); Lefranc M. P. et al., Dev. Comp. Immunol., 27: 55-77 (2003); and Honegger and Plückthun, J. Mol. Biol., 309: 657-670 (2001). “CDR” herein may be labeled and defined in a manner well known in the art, including, but not limited to, Kabat numbering scheme, Chothia numbering scheme, or IMGT numbering scheme; the tool sites used include, but are not limited to, AbRSA site (website: cao.labshare.cn/AbRSA/cdrs.php), abYsis site (website: abysis.org/abysis/sequence_input/key_annotation/key_annotation.cgi), and IMGT site (website:.imgt.org/3Dstructure-DB/cgi/DomainGapAlign.cgi#results). The CDR herein includes overlaps and subsets of amino acid residues defined in different ways.

The term “Kabat numbering scheme” herein generally refers to the immunoglobulin alignment and numbering scheme proposed by Elvin A. Kabat (see, e.g., Kabat et al., Sequences of Proteins of Immunological Interest, 5th Ed. Public Health Service, National Institutes of Health, Bethesda, Md., 1991).

The term “Chothia numbering scheme” herein generally refers to the immunoglobulin numbering scheme proposed by Chothia et al., which is a classical rule for identifying CDR region boundaries based on the position of structural loop regions (see, e.g., Chothia & Lesk (1987) J. Mol. Biol. 196: 901-917; Chothia et al., (1989) Nature 342: 878-883).

The term “IMGT numbering scheme” herein generally refers to a numbering scheme based on the international ImMunoGeneTics information system (IMGT) initiated by Lefranc et al., see Lefranc et al., Dev. Comparat. Immunol. 27: 55-77, 2003.

The term “heavy chain constant region” herein refers to the carboxyl-terminal portion of an antibody heavy chain that is not directly involved in the binding of the antibody to an antigen, but exhibits effector functions, such as interaction with an Fc receptor, which has a more conserved amino acid sequence relative to the variable domain of the antibody. The “heavy chain constant region” at least comprises: a CH1 domain, a hinge region, a CH2 domain, a CH3 domain, or a variant or fragment thereof. The “heavy chain constant region” includes a “full-length heavy chain constant region” having a structure substantially similar to that of a natural antibody constant region, and a “heavy chain constant region fragment” including only a portion of the full-length heavy chain constant region. Exemplarily, a typical “full-length antibody heavy chain constant region” consists of the CH1 domain-hinge region-CH2 domain-CH3 domain. When the antibody is IgE, it further comprises a CH4 domain; and when the antibody is a heavy chain antibody, it does not comprise a CH1 domain. Exemplarily, a typical “heavy chain constant region fragment” may be selected from an Fc domain and a CH3 domain.

The term “light chain constant region” herein refers to the carboxyl-terminal portion of an antibody light chain that is not directly involved in the binding of the antibody to an antigen. The light chain constant region may be selected from a constant κ domain and a constant λ domain.

The term “Fc region” herein is used to define the C-terminal region of an antibody heavy chain comprising at least a portion of the constant region. The “Fc region” includes Fc regions of native sequences and variant Fc regions. Exemplarily, the human IgG heavy chain Fc region may extend from Cys226 or Pro230 to the carboxyl-terminus of the heavy chain. However, an antibody produced by the host cell may undergo post-translational cleavage, cleaving off one or more (particularly one or two) amino acids from the C-terminus of the heavy chain. Therefore, the antibody generated by a host cell by the expression of a specific nucleic acid molecule encoding a full-length heavy chain may include a full-length heavy chain, or may include cleaved variants of the full-length heavy chain. It may be such a situation when the final two C-terminal amino acids of the heavy chain are glycine (G446) and lysine (K447, numbered according to the Kabat EU index). Therefore, the C-terminal lysine (Lys447), or the C-terminal glycine (Gly446) and lysine (Lys447) of the Fc region may be present or absent.

The IgG Fc region comprises IgG CH2 and IgG CH3 domains, and optionally, may further comprise an intact or partial hinge region, but exclude a CH1 domain. The “CH2 domain” of the human IgG Fc region typically extends from an amino acid residue at about position 231 to an amino acid residue at about position 340. In one embodiment, a carbohydrate chain is attached to the CH2 domain. The CH2 domain herein may be a CH2 domain of a native sequence or a variant CH2 domain. The “CH3 domain” comprises the residues in the Fc region at the C-terminus of the CH2 domain (i.e., from an amino acid residue at about position 341 to an amino acid residue at about position 447 of the IgG). The CH3 region herein may be a CH3 domain of a native sequence or a variant CH3 domain (e.g., a CH3 domain having a “bulge” (“knob”) introduced in one strand and a “cavity” (“hole”) correspondingly introduced in the other strand; see U.S. Pat. No. 5,821,333, which is explicitly incorporated herein by reference). As described herein, such variant CH3 domain may be used to promote the heterodimerization of two non-identical antibody heavy chains.

Unless otherwise specified herein, the numbering of amino acid residues in the Fc region or constant region conforms to the EU numbering scheme, also known as the EU index, as described in Kabat et al., Sequences of Proteins of Immunological Interest, 5th Ed., Public Health Service, National Institutes of Health, Bethesda, M D, 1991.

The term “conservative amino acid” herein generally refers to amino acids that belong to the same class or have similar characteristics (e.g., charge, side chain size, hydrophobicity, hydrophilicity, backbone conformation, and rigidity). Exemplarily, the amino acids in each of the following groups are conservative amino acid residues of each other, and substitutions of amino acid residues within the groups are conservative amino acid substitutions:
1) alanine (A), serine (S), and threonine (T);
2) aspartic acid (D) and glutamic acid (E);
3) asparagine (N) and glutamine (Q);
4) arginine (R), lysine (K), and histidine (H);
5) isoleucine (I), leucine (L), methionine (M), and valine (V); and
6) phenylalanine (F), tyrosine (Y), and tryptophan (W).

The terms “percent (%) sequence identity” and “percent (%) identity” herein are used interchangeably, referring to the percentage of identity between amino acid (or nucleotide) residues of a candidate sequence and amino acid (or nucleotide) residues of a reference sequence after aligning the sequences and introducing gaps, if necessary, to achieve maximum percent sequence identity (e.g., gaps may be introduced in one or both of the candidate sequence and the reference sequence for optimal alignment, and non-homologous sequences may be omitted for the purpose of comparison). Alignment may be carried out in a variety of ways well known to those skilled in the art for the purpose of determining percent sequence identity, for example, using publicly available computer software such as BLAST, ALIGN, or Megalign (DNASTAli) software. Those skilled in the art can determine appropriate parameters for measuring alignment, including any algorithms required to achieve maximum alignment of the full length of the aligned sequences. For example, a reference sequence aligned for comparison with a candidate sequence may show that the candidate sequence exhibits 50% to 100/6 sequence identity over the full length of the candidate sequence or a selected portion of contiguous amino acid (or nucleotide) residues of the candidate sequence. The length of the candidate sequence aligned for the purpose of comparison may be, e.g., at least 30% (e.g., 30%, 40%, 50%, 60%, 70%, 80%, 90%, or 100%) of the length of the reference sequence. When a position in the candidate sequence is occupied by the same amino acid (or nucleotide) residue at the corresponding position in the reference sequence, then the molecules are identical at that position.

The term “chimeric antigen receptor (CAR)” herein refers to an artificial cell surface receptor engineered to be expressed on an immune effector cell and specifically bound to an antigen, which comprises at least (1) an extracellular antigen-binding domain, e.g., a variable heavy or light chain of an antibody, (2) a transmembrane domain that anchors the CAR into the immune effector cell, and (3) an intracellular signaling domain. The CAR is capable of redirecting T cells and other immune effector cells to a selected target, e.g., a cancer cell, in a non-MHC-restricted manner using the extracellular antigen-binding domain.

The term “nucleic acid” herein includes any compound and/or substance that comprises a polymer of nucleotides. Each nucleotide consists of a base, in particular a purine or pyrimidine base (i.e., cytosine (C), guanine (G), adenine (A), thymine (T), or uracil (U)), a sugar (i.e., deoxyribose or ribose), and a phosphate group. Generally, a nucleic acid molecule is described as a sequence of bases, whereby the bases represent the primary structure (linear structure) of the nucleic acid molecule. The sequence of bases is generally expressed as 5′ to 3′. In this context, the term “nucleic acid molecule” encompasses deoxyribonucleic acid (DNA), including, e.g., complementary DNA (cDNA) and genomic DNA; ribonucleic acid (RNA), in particular in the synthetic form of messenger RNA (mRNA), DNA or RNA; and polymers comprising a mixture of two or more of these molecules. The nucleic acid molecule may be linear or cyclic. Furthermore, the term “nucleic acid molecule” includes both sense and antisense strands, as well as single- and double-stranded forms. Moreover, the nucleic acid molecules described herein may contain naturally occurring or non-naturally occurring nucleotides. Examples of non-naturally occurring nucleotides include modified nucleotide bases having derived sugar or phosphate backbone linkages or chemically modified residues. The nucleic acid molecule also encompasses DNA and RNA molecules suitable for use as vectors for direct expression of the antibodies of the present invention in vitro and/or in vivo, e.g., in a host or patient. Such DNA (e.g., cDNA) or RNA (e.g., mRNA) vectors may be unmodified or modified. For example, mRNA can be chemically modified to enhance the stability of the RNA vector and/or the expression of the encoded molecule such that the mRNA can be injected into a subject to produce antibodies in vivo (see, e.g., Stadler el al., Nature Medicine 2017, published online, Jun. 12, 2017, doi: 10.1038/nm.4356 or EP 2 101 823 B1). An “isolated” nucleic acid herein refers to a nucleic acid molecule that has been separated from components of its natural environment. The isolated nucleic acid includes a nucleic acid molecule contained in a cell that generally contains the nucleic acid molecule, but the nucleic acid molecule is present extrachromosomally or at a chromosomal location different from its natural chromosomal location.

The term “vector” herein includes nucleic acid vectors, e.g., DNA vectors (e.g., plasmids), RNA vectors, viruses, or other suitable replicons (e.g., viral vectors). Various vectors have been developed for the delivery of polynucleotides encoding foreign proteins into prokaryotic or eukaryotic cells. The expression vector of the present invention contains polynucleotide sequences as well as additional sequence elements, e.g., for expressing proteins and/or integrating these polynucleotide sequences into the genome of mammalian cells. Some vectors that may be used to express the antibody and antibody fragment of the present invention include plasmids comprising regulatory sequences (e.g., promoter and enhancer regions) that direct gene transcription. Other useful vectors for expressing the antibody and antibody fragment contain polynucleotide sequences that enhance the rate of translation of these genes or improve the stability or nuclear export of mRNA produced by gene transcription. These sequence elements include, for example, 5′ and 3′ untranslated regions, internal ribosome entry sites (IRESs), and polyadenylation signal sites, so as to direct the effective transcription of the gene carried on the expression vector. The expression vector of the present invention may also contain a polynucleotide encoding a marker for selecting cells comprising such a vector. Examples of suitable markers include genes encoding resistance to antibiotics (e.g., ampicillin, chloramphenicol, kanamycin, or nourseothricin).

The term “host cell” herein refers to a cell into which an exogenous nucleic acid has been introduced, including the progeny of such a cell. Host cells include “transformants” and “transformed cells”, which include primary transformed cells and progenies derived therefrom, regardless of the number of passages. Progenies may not be exactly the same as parent cells in terms of nucleic acid content, and may contain mutations. Mutant progenies having the same function or biological activity that are screened or selected from the primary transformed cells are included herein.

The step of transforming host cells with recombinant DNA described in the present invention may be performed using a conventional technique well known to those skilled in the art. The obtained transformants may be cultured by a conventional method, and express the polypeptide encoded by the gene of the present invention. The medium used in culture may be selected from various conventional media depending on the host cells used. The host cells are cultured under conditions suitable for their growth.

The term “pharmaceutical composition” herein refers to a formulation that exists in a form allowing the biological activity of the active ingredient contained therein to be effective, and does not contain additional ingredients having unacceptable toxicity to a subject to which the pharmaceutical composition is administered.

The terms “subject” and “patient” herein refer to an organism that receives treatment for a particular disease or disorder (e.g., a cancer or an infectious disease) as described herein. Examples of the subject and the patient include mammals, such as human, primate, pig, goat, rabbit, hamster, cat, dog, guinea pig, members of the bovine family (such as cattle, bison, buffalo, elk, yak, etc.), cattle, sheep, horse, bison, etc., that are receives treatment for a disease or disorder (e.g., a cell proliferative disorder, such as a cancer or an infectious disease).

The term “treatment” herein refers to a surgical or therapeutic treatment for the purpose of preventing or slowing (reducing) the progression of an undesirable physiological or pathological change, such as a cell proliferative disorder (such as a cancer or an infectious disease), in a subject being treated. Beneficial or desired clinical results include, but are not limited to, alleviation of symptoms, decrease of severity of disease, stabilization (i.e., not worsening) of state of disease, delay or slowing of disease progression, amelioration or palliation of state of disease, and remission (whether partial or total), whether detectable or undetectable. Subjects in need of treatment include those already with a disorder or disease, as well as those who are susceptible to a disorder or disease or those who intend to prevent a disorder or disease. When referring to terms such as slowing, alleviation, decrease, palliation, and remission, their meanings also include elimination, disappearance, nonoccurrence, etc.

The term “effective amount” herein refers to an amount of a therapeutic agent that is effective to prevent or alleviate symptoms of a disease or the progression of the disease when administered to a cell, tissue, or subject alone or in combination with another therapeutic agent. “Effective amount” also refers to an amount of a compound that is sufficient to alleviate symptoms, e.g., to treat, cure, prevent, or alleviate a related medical disorder, or to increase the rate at which such disorder is treated, cured, prevented, or alleviated. When the active ingredient is administered alone to an individual, a therapeutically effective dose refers to the amount of the ingredient alone. When a combination is used, a therapeutically effective dose refers to the combined amounts of the active ingredients that produce the therapeutic effect, whether administered in combination, sequentially, or simultaneously.

The term “cancer” herein refers to or describes a physiological condition in mammals that is typically characterized by unregulated cell growth. Included in this definition are benign and malignant cancers. The term “tumor” or “neoplasm” herein refers to all neoplastic cell growth and proliferation, whether malignant or benign, and all pre-cancerous and cancerous cells and tissues. The terms “cancer” and “tumor” are not mutually exclusive when referred to herein.

### BRIEF DESCRIPTION OF THE DRAWINGS

The foregoing and other aspects of the present invention will be clearly illustrated by the following detailed description of the present invention and the accompanying drawings. The accompanying drawings herein are intended to illustrate certain preferred embodiments of the present invention. However, it should be understood that the present invention is not limited to the specific embodiments disclosed.

FIG. 1 shows assay results for human CD33-V-his and CD33-C2-his protein samples by SDS-PAGE reducing gel and non-reducing gel: 1-hCD33-V-his (non-reducing); 2-hCD33-V-his (reducing); 3-hCD33-C2-his (non-reducing); 4-hCD33-C2-his (reducing); and M-marker.

FIG. 2A shows assay results for binding reactions of control antibodies with a human CD33-his protein by ELISA.

FIG. 2B shows assay results for binding reactions of control antibodies with a human CD33-V-his protein by ELISA.

FIG. 2C shows assay results for binding reactions of control antibodies with a human CD33-C2-his protein by ELISA.

FIG. 3 shows FACS results of CD33 expression levels of in U937 cells using antibody SGN-33.

FIG. 4 shows FACS screening results of CHO-K1 cells transfected with human CD33 protein.

FIG. 5 shows FACS screening results of HEK293T cells transfected with monkey CD33 protein.

FIG. 6A shows serum antibody titers of llamas after immunization with human CD33 protein assayed by ELISA.

FIG. 6B shows serum antibody titers of llamas after immunization with human CD33 protein assayed by FACS.

FIG. 7A and FIG. 7B show assay results for binding reactions of VHH antibodies with a human CD33-his protein by ELISA.

FIG. 8A and FIG. 8B show assay results for binding reactions of VHH antibodies with CHO-K1-human CD33 by FACS.

FIG. 8C and FIG. 8D show assay results for binding reactions of VHH antibodies with U937 by FACS.

FIG. 9A and FIG. 9B show assay results for binding reactions of VHH antibodies with a murine CD33-His protein by ELISA.

FIG. 9C and FIG. 9D show assay results for binding reactions of VHH antibodies with a monkey CD33-His protein by ELISA.

FIG. 10A and FIG. 10B show assay results for binding reactions of VHH antibodies with HEK293T-monkey CD33 by FACS.

FIG. 11A and FIG. 11C show assay results for binding reactions of VHH antibodies with a human CD33-V-his protein by ELISA.

FIG. 11B and FIG. 11D show assay results for binding reactions of VHH antibodies with a human CD33-C2-his protein by ELISA.

FIG. 12A and FIG. 12B show the inhibition rate of VHH antibodies by a competitive ELISA method.

FIG. 13A and FIG. 13B show results of ELISA competition between VHH antibodies and Biotin-C33B904.

FIG. 14 shows classification of epitopes of VHH antibodies.

FIG. 15A to FIG. 15E show assay results for binding reactions of VHH humanized antibodies with a human CD33-his protein by ELISA.

FIG. 16A to FIG. 16E show assay results for binding reactions of VHH humanized antibodies with CHO-K1-human CD33 by FACS.

FIG. 17A to FIG. 17E show assay results for binding reactions of VHH humanized antibodies with U937 by FACS.

FIG. 18A to FIG. 18E show assay results for binding reactions of VHH humanized antibodies with a murine CD33-his protein by ELISA.

FIG. 18F to FIG. 18J show assay results for binding reactions of VHH humanized antibodies with a monkey CD33-his protein by ELISA.

FIG. 19A to FIG. 19E show assay results for binding reactions of VHH humanized antibodies with HEK293T-monkey CD33 by FACS.

FIG. 20A to FIG. 20E show assay results for binding reactions of VHH humanized antibodies with a human CD33-V-his protein by ELISA.

FIG. 20F to FIG. 20J show assay results for binding reactions of VHH humanized antibodies with a human CD33-C2-his protein by ELISA.

### DETAILED DESCRIPTION

The present invention will be described in detail below with reference to examples and the accompanying drawings. The accompanying drawings herein are intended to illustrate some preferred embodiments of the present invention. However, it should be understood that the present invention is not limited to the specific embodiments disclosed, or that the specific embodiments disclosed are not to be considered as a limitation to the scope of the present invention. Experimental procedures without specified conditions in the examples are conducted according to conventional conditions or conditions recommended by the manufacturers. Reagents or instruments without specified manufacturers used herein are conventional products that are commercially available.

### Example 1: Preparation of Control Antibodies and CD33 Proteins

### 1.1 Preparation of Control Antibodies

The extracellular region of CD33 protein has a conserved V-set immunoglobulin-like domain and a variable C2-set domain. SGN-33 and C33B904 are antibodies recognizing human CD33, wherein the antigen-binding epitope of SGN-33 is located at the V-set domain, and the antigen-binding epitope of C33B904 is located at the C2-set domain. The sequences of the heavy chain variable region and the light chain variable region of SGN-33 are obtained according to the U.S. Pat. No. 9,453,046, and the sequences of the heavy chain variable region and the light chain variable region of C33B904 are obtained according to the patent US20190382481A1. VH and VL of the antibody SGN-33, which recognizes human CD33, and human IgG1 Fc were linked in an order from N-terminus to C-terminus, wherein the VH and the VL were linked by 3 GGGGS (SEQ ID NO: 231) linkers to form scFv-hFc; VH and VL of the antibody C33B904 which recognizes human CD33 were formed into an intact antibody form of human IgG1. The information on the corresponding amino acid sequences is shown in Table 1 below, with the antibody variable region sequences underlined and italicized. The corresponding nucleotide sequences were separately cloned into a pTT5 vector (implemented by General Biosystems (Anhui) Corporation Limited), and plasmids were prepared according to established standard molecular biology methods. See Sambrook, J., Fritsch, E. F., and Maniatis, T. (1989). Molecular Cloning: A Laboratory Manual, Second Edition (Plainview, New York: Cold Spring Harbor Laboratory Press). HEK293E cells (purchased from Suzhou Yiyan Biotech Co., Ltd.) were transiently transfected with the expression vectors according to PEI (purchased from Polysciences, Cat. No. 24765-1) instructions, cultured at 37° C. for 5 consecutive days using FreeStyle™ 293 (Thermofisher scientific, Cat. No. 12338018), and centrifuged to remove cell components to obtain culture supernatants containing the antibodies. The culture supernatants were each loaded on a Protein A chromatography column (the Protein A packing AT Protein A Diamond and chromatography column BXK16/26 were purchased from Bestchrom, with Cat. Nos. of AA0273 and B-1620, respectively), washed with phosphate-buffered saline (PBs; pH 7.4), then washed with 20 mM PB, 1 M NaCL (pH 7.2), and finally subjected to elution with a citrate buffer at pH 3.4. An Fc-tagged antibody eluted from the Protein A chromatography column was collected, neutralized with 1/10 volumes of 1 M Tris at pH 8.0, and dialyzed with PBS at 4° C. overnight. The dialyzed protein was subjected to sterile filtration through a 0.22 μM filter membrane, subpackaged, and stored at −80′° C.

TABLE 1
Sequence information of anti-human CD33 antibodies
SGN-33 and C33B904

| Sequence | | |
|---|---|---|
| name | Sequence No. | Amino acid sequence |
| SGN-33 | SEQ ID NO: 1 | QVQLVQSGAEVKKPGASVKVSCKASGYTFTNYDINWVRQAPGQGLE |
| scFv- | | WIGWIYPGDGSTKYNEKFKAKATLTADTSTSTAYMELRSLRSDDTA |
| hIgG1Fc | | VYYCASGYEDAMDYWGQGTTVTVSSGGGGSGGGGGGGGSDIQMTQS |
| | | PSSLSASVGDRVTINCKASQDINSYLSWFQQKPGKAPKTLIYRANR |
| | | LVDGVPSRFSGSGSGQDYTLTISSLQPEDFATYYCLQYDEFPLTFG |
| | | GGTKVEIKEPKSADKTHTCPPCPAPELLGGPSVFLFPPKPKDTLMI |
| | | SRTPEVTCVVVDVSHEDPEVKFNWYVDGVEVHNAKTKPREEQYN |
| | | STYRVVSVLTVLHQDWLNGKEYKCKVSNKALPAPIEKTISKAKG |
| | | QPREPQVYTLPPSREEMTKNQVSLTCLVKGFYPSDIAVEWESNG |
| | | QPENNYKTTPPVLDSDGSFFLYSKLTVDKSRWQQGNVFSCSVMH |
| | | EALHNHYTQKSLSLSPGK |
| C33B904 | SEQ ID NO: 2 | EVQLVESGGGLVQPGRSLRLSCAASGFTEDDYAMHWVRQAPGKGLE |
| hIgG1- | | WVSGIGWSGGSIVYADSVKGRFTISRDNAKNSLYLQMNSLRAEDTA |
| heavy | | LYYCAKDSPYGDFFDYWGQGTLVTVSSASTKGPSVFPLAPSSKSTS |
| chain | | GGTAALGCLVKDYFPEPVTVSWNSGALTSGVHTFPAVLQSSGLY |
| | | SLSSVVTVPSSSLGTQTYICNVNHKPSNTKVDKKVEPKSCDKTH |
| | | TCPPCPAPELLGGPSVFLFPPKPKDTLMISRTPEVTCVVVDVSHE |
| | | DPEVKFNWYVDGVEVHNAKTKPREEQYNSTYRVVSVLTVLHQ |
| | | DWLNGKEYKCKVSNKALPAPIEKTISKAKGQPREPQVYTLPPSR |
| | | EEMTKNQVSLTCLVKGFYPSDIAVEWESNGQPENNYKTTPPVLD |
| | | SDGSFFLYSKLTVDKSRWQQGNVFSCSVMHEALHNHYTQKSLS |
| | | LSPGK |
| C33B904 | SEQ ID NO: 3 | DIVMTQSPDSLAVSLGERATINCKSSQTVFYSSNNKNYLAWYQQKP |
| hIgG1- | | GQPPKLLISWASTRKSGVPDRESGSGSGTDETLTVSSLQAEDVAVY |
| light | | YCQHYYSTPYTFGQGTKLEIKRTVAAPSVFIFPPSDEQLKSGTASV |
| chain | | VCLLNNFYPREAKVQWKVDNALQSGNSQESVTEQDSKDSTYSLSST |
| | | LTLSKADYEKHKVYACEVTHQGLSSPVTKSENRGEC |

### 1.2 Preparation of Human CD33-V-his and Human CD33-C2-his Proteins

A protein comprising the amino acid sequence Asp 18-His 259 encoding the extracellular domain (ECD) of human CD33 protein was purchased from ACROBiosystems (Cat. No. CD3-H5226) and was designated human CD33-His.

The nucleotide sequences encoding the amino acid sequences of human CD33-V domain Pro19-Ser135 (SEQ ID NO: 4) and C2 domain Pro145-Gln228 (SEQ ID NO: 5) with His tags were separately cloned into a pTT5 vector (implemented by General Biosystems (Anhui) Corporation Limited), and plasmids were prepared according to established standard molecular biology methods. The information on the corresponding amino acid sequences is shown in Table 2 below. See Sambrook, J., Fritsch, E. F., and Maniatis, T. (1989). Molecular Cloning: A Laboratory Manual, Second Edition (Plainview, New York: Cold Spring Harbor Laboratory Press). HEK293E cells (purchased from Suzhou Yiyan Biotech Co., Ltd.) were transiently transfected (PEI, Polysciences, Cat. No. 24765-1) and expanded at 37° C. using FreeStyle™ 293 (Thermofisher scientific, Cat. No. 12338018). After 6 days, the cell cultures were collected and centrifuged to remove cell components to obtain culture supernatants containing the V domain and the C2 domain of the human CD33 protein. The culture supernatants were each loaded on a nickel ion affinity chromatography column HisTrap™ Excel (GE Healthcare, Cat. No. GE17-3712-06), and meanwhile, the changes in UV absorption value (A280 nm) were monitored with an ultraviolet (UV) detector. After loading, the nickel ion affinity chromatography column was washed with 20 mM PB, 0.5 M NaCl (pH 7.4) until the UV absorption value returned to the baseline, and then gradient elution (2%, 4%, 8%, 16%, 50%, and 100%) was performed with buffer A (20 mM PB, 0.5 M NaCl (pH 7.4)) and buffer B (20 mM PB, 0.5 M NaCl, 500 mM imidazole). A His-tagged human CD33 protein eluted from the nickel ion affinity chromatography column was collected and dialyzed with phosphate-buffered saline (PBS; pH 7.4) in a refrigerator at 4° C. overnight. The dialyzed protein was subjected to sterile filtration through a 0.22 μM filter membrane, subpackaged, and stored at −80° C. to obtain a purified human CD33 protein. The target bands of the sample as assayed by SDS-PAGE reducing gel and non-reducing gel are shown in FIG. 1.

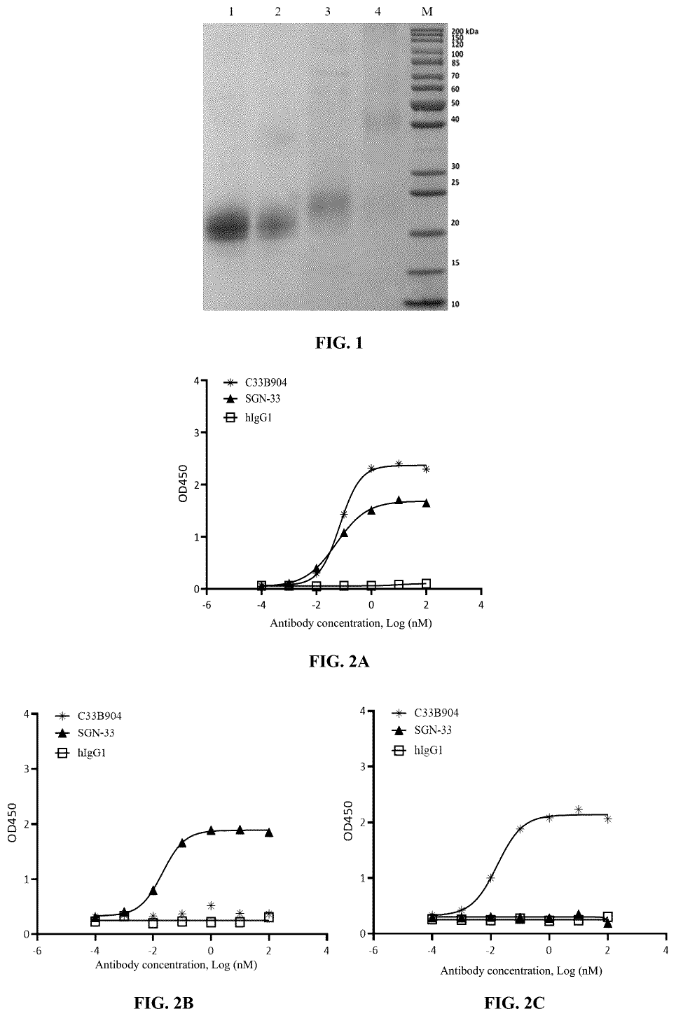

The above CD33 protein was assayed by ELISA using positive control antibodies recognizing different epitopes, and a negative control antibody hIgG1 was an anti-hel-hIgG1 antibody (purchased from Biointron, Cat. No. B117901) against hen egg lysozyme. The assay results are shown in Tables 3-5 and FIGS. 2A-2C, which show that both SGN-33 and C33B3904 can bind to human CD33-his protein, SGN-33 can bind to human CD33-V-his protein, while C33B904 can bind to human CD33-C2-his protein. The assay results are consistent with the binding epitopes reported in the literature, indicating that proteins with the above binding activities have been prepared.

TABLE 2
Amino acid sequences of human CD33 protein extracellular
domains, V-domain and C2-domain

| Sequence | | |
|---|---|---|
| name | Sequence No. | Amino acid sequence |
| Human CD33 | SEQ ID NO: 4 | DPNFWLQVQESVTVQEGLCVLVPCTFFHPIPYYDKNSPVHGYW |
| protein | | FREGAIISRDSPVATNKLDQEVQEETQGRFRLLGDPSRNNCSL |
| V-domain | | SIVDARRRDNGSYFFRMERGSTKYSYKSPQLSVHVTDLTHRGS |
| Human CD33 | SEQ ID NO: 5 | VHVTDLTHRPKILIPGTLEPGHSKNLTCSVSWACEQGTPPIFS |
| protein | | WLSAAPTSLGPRTTHSSVLIITPRPQDHGTNLTCQVKFAGAGV |
| C2-domain | | TTERTIQLNVTYVPQNPTTGIFPGDGSGKQETRAGVVHGS |

TABLE 3
Assay results for binding reactions of control
antibodies with human CD33-his protein by ELISA

| OD450 | | | | | | | | |
|---|---|---|---|---|---|---|---|---|
| Antibody | Antibody concentration (nM) | | | | | | | |
| name | 100 | 10 | 1 | 0.1 | 0.01 | 0.001 | 0.0001 | Blank |
| C33B904 | 2.30 | 2.40 | 2.31 | 1.43 | 0.30 | 0.08 | 0.05 | 0.05 |
| SGN-33 | 1.65 | 1.71 | 1.51 | 1.08 | 0.40 | 0.11 | 0.06 | 0.05 |
| hIgG1 | 0.10 | 0.08 | 0.06 | 0.06 | 0.05 | 0.06 | 0.06 | 0.08 |

TABLE 4
Assay results for binding reactions of control antibodies
with human CD33-V-his protein by ELISA

| OD450 | | | | | | | | |
|---|---|---|---|---|---|---|---|---|
| Antibody | Antibody concentration (nM) | | | | | | | |
| name | 100 | 10 | 1 | 0.1 | 0.01 | 0.001 | 0.0001 | Blank |
| C33B904 | 0.37 | 0.38 | 0.52 | 0.37 | 0.33 | 0.35 | 0.32 | 0.35 |
| SGN-33 | 1.85 | 1.90 | 1.89 | 1.66 | 0.80 | 0.41 | 0.31 | 0.32 |
| hIgG1 | 0.31 | 0.22 | 0.22 | 0.23 | 0.20 | 0.33 | 0.23 | 0.31 |

TABLE 5
Assay results for binding reactions of control antibodies
with human CD33-C2-his protein by ELISA

| OD450 | | | | | | | | |
|---|---|---|---|---|---|---|---|---|
| Antibody | Antibody concentration (nM) | | | | | | | |
| name | 100 | 10 | 1 | 0.1 | 0.01 | 0.001 | 0.0001 | Blank |
| C33B904 | 2.06 | 2.23 | 2.08 | 1.88 | 1.00 | 0.42 | 0.33 | 0.34 |
| SGN-33 | 0.19 | 0.35 | 0.28 | 0.29 | 0.30 | 0.29 | 0.29 | 0.32 |
| hIgG1 | 0.30 | 0.24 | 0.23 | 0.27 | 0.24 | 0.25 | 0.26 | 0.28 |

### Example 2: Identification of Endogenous Expression Cell Strain and Preparation of Over-Expression Cell Strain

### 2.1 Identification of Cell Strain Endogenously Expressing CD33

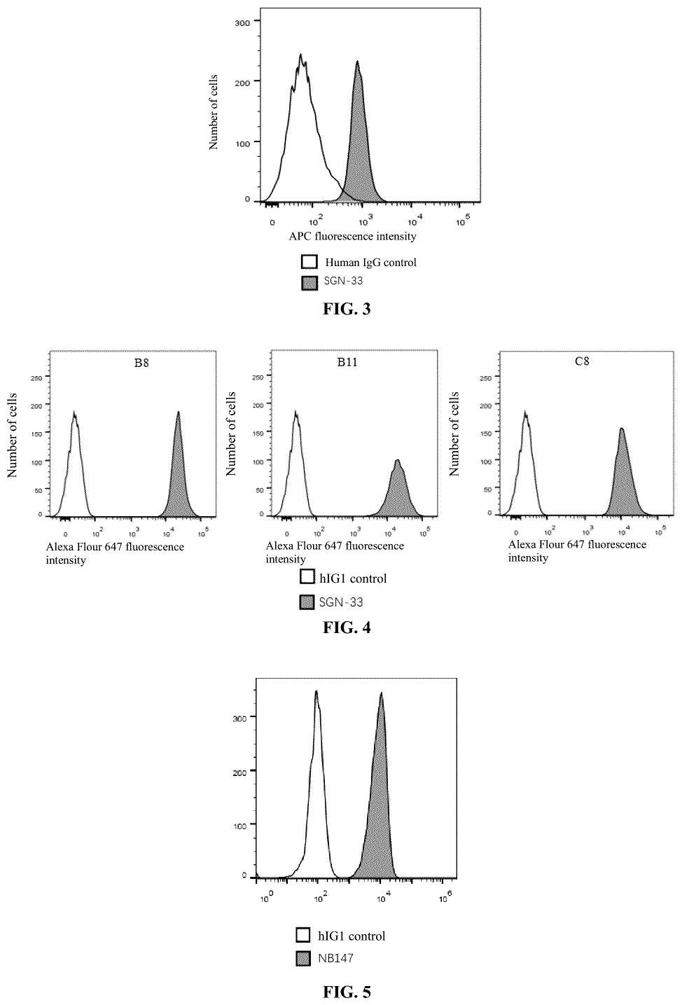

U937 cells were expanded to a logarithmic growth phase in a T-25 cell culture flask, and the medium supernatant was discarded after centrifugation. The cell pellet was washed twice with PBS, and incubated for 15 min with a buffer containing human Fc Block (purchased from BD, Cat. No. 564220). The results were assayed and analyzed by FACS (FACS Canto™, purchased from BD Biosciences) using the antibody SGN-33 as a primary antibody and an APC-labeled secondary antibody (purchased from Biolegend, Cat. No. 409306). The analysis results are shown in Table 6 and FIG. 3, which show that U937 cells can bind to SGN-33.

TABLE 6
Assay results for endogenous cell line U937 cells by FACS

| | | Mean fluorescence intensity of cells | |
|---|---|---|---|
| No. | Antibody name | IgG subtype control | CD33 antibody |
| 1 | SGN-33 | 114 | 1081 |

2.2 Preparation of CHO-K1 Monoclonal Cell Strains Stably Transfected with Human CD33

A nucleotide sequence encoding a full-length amino acid sequence of human CD33 (NCBI. XP_011525834.1) was cloned into a pcDNA3.1 vector, and a plasmid was prepared (implemented by General Biosystems (Anhui) Corporation Limited). After plasmid transfection (Lipofectamine® 3000 Transfection Kit, purchased from Invitrogen, Cat. No. L3000-015) of a CHO-K1 cell line (purchased from Chinese Academy of Sciences, Shanghai Institutes for Biological Sciences), the cells were selectively cultured in a DMEM/F12 medium containing 10 μg/mL puromycin and 10% (w/w) fetal bovine serum for 2 weeks, and positive monoclonal cells were sorted on a flow cytometer FACS AriaII (BD Biosciences) using the antibody SGN-33 as a primary antibody and an Alexa Fluor 647-labeled secondary antibody (Jackson, Cat. No. 109605088), added into a 96-well plate, and cultured at 37° C. with 5% (v/v) CO₂. After about 2 weeks, some of the single clones were selected for expansion. The amplified clones were screened by flow cytometry. Monoclonal cell lines with better growth and higher fluorescence intensity were selected for further expansion and cryopreserved in liquid nitrogen.

The specific results of selection are shown in Table 7 and FIG. 4, in which the IgG subtype control was a human IgG control. Table 7 shows that a series of CHO-K1 monoclonal cell lines with positive expression of human CD33 have been prepared. In FIG. 4, the abscissa represents the fluorescence intensity of the cells, and the ordinate represents the number of the cells. The results in FIG. 4 indicate that CHO-K1-human CD33 B8, CHO-K1-human CD33 B11, and CHO-K1-human CD33 C8 were cell strains with high-level expression of human CD33.

TABLE 7
Assay results for CHO-K1 cell lines stably transfected
with human CD33 protein by FACS

| | Clone No. of stably | Mean fluorescence intensity of cells | |
|---|---|---|---|
| No. | transfected cell line | IgG subtype control | CD33 antibody |
| 1 | CHO-K1-human CD33 B8 | 30.9 | 25413 |
| 2 | CHO-K1-human CD33 B11 | 30.9 | 23912 |
| 3 | CHO-K1-human CD33 C8 | 30.9 | 14175 |

2.3 Preparation of HEK293T Cell Strain Stably Transfected with Monkey CD33

A nucleotide sequence encoding a full-length amino acid sequence (NCBI: XP_014980179.1) of monkey CD33 was cloned into a pcDNA3.1 vector (purchased from Thermofisher scientific), and a plasmid was prepared. After plasmid transfection of an HEK293T cell line with FuGENE® HD (Promega, Cat. No. E2311), the cells were selectively cultured in a DMEM medium containing 10 μg/mL puromycin and 10% (w/w) fetal bovine serum for 2 weeks, subcloned in a 96-well culture plate by a limiting dilution method, and cultured at 37° C. with 5% (v/v) CO₂. About 2 weeks later, some of the polyclonal wells were selected for amplification in a 6-well plate. NB147 was a polyclonal antibody (prepared in Example 3) obtained from the serum of llama immunized with the human CD33 protein. The amplified clones were screened by flow cytometry using NB147 as a positive control, and monoclonal cell lines with better growth and higher fluorescence intensity were selected for further expansion and cryopreserved in liquid nitrogen. As shown in Table 8 and FIG. 5, the assay results for the HEK293T cell strain by flow cytometry shows that single positive cell peaks of the overexpressed monkey CD33 after puromycin screening can be used to assay the cross-activity of antibodies.

TABLE 8
Assay results for HEK293T cell line stably transfected
with monkey CD33 protein by FACS

| | | Mean fluorescence intensity of cells | |
|---|---|---|---|
| | Clone No. of stably | Secondary | |
| No. | transfected cell line | antibody control | NB147 llama serum |
| 1 | HEK293T-monkey CD33 | 99 | 9315 |

### Example 3: Screening of Single Domain Antibodies VHH Against CD33

### 3.1 Llama Immunization and Serum Titer Assay

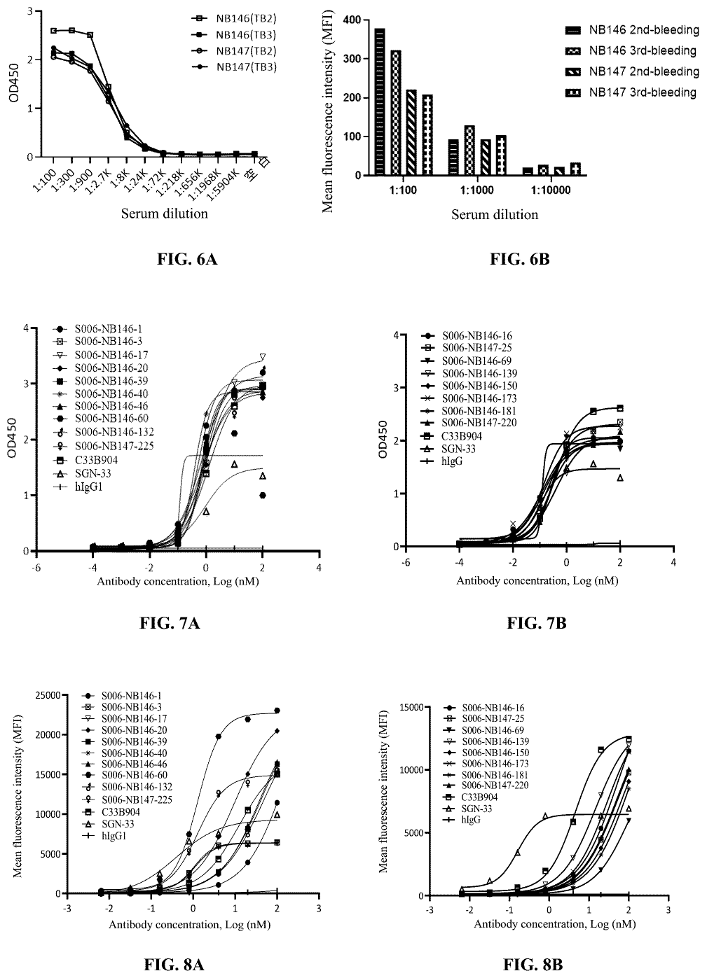

Two llamas were selected for immunization, with each llama being immunized four times at an interval of 3 weeks; the first and second immunizations were performed using human CD33(Asp18-His259)-Pol-his protein purchased from ACROBiosystems (Cat. No. CD3-H5226); the third immunization was performed using human CD33 (Asp18-His259)-Fc(Pro100-Lys330) protein purchased from ACROBiosystems (Cat. No. CD3-H5257); and the fourth immunization was performed using CHO-K1-human CD33-B8 prepared in Example 2. Peripheral blood was collected and serum was separated after the third and fourth immunizations, and then the titers and specificity of antibodies against human CD33 (hCD33) in the serum were assayed using enzyme-linked immunosorbent assay (ELISA) and flow cytometry assay (FACS). The results are shown in FIGS. 6A-6B and Table 9. Table 9 shows that the sera from the llamas immunized with human CD33 exhibited different degrees of binding to the immunogen, showing antigen-antibody reactions, wherein the maximum dilution factor was about 5.9 million. In this table, the blank control is 1% (w/w) BSA, and the batches refer to llama sera on day seven after the third (TB2, 2nd-bleeding) and fourth (TB3, 3rd-bleeding) immunizations. Data in the table are OD450 nm values. The results show that NB147 llama serum can bind to murine CD33 protein and can serve as a positive control antibody for subsequent cross-reaction experiments.

TABLE 9
Serum antibody titers of llamas after immunization
with a hCD33 protein assayed by ELISA
OD450nm

| | Batch | | | |
|---|---|---|---|---|
| Serum dilution | NB146(TB2) | NB146(TB3) | NB147(TB2) | NB147(TB3) |
| 1:100 | 2.60 | 2.14 | 2.05 | 2.25 |
| 1:300 | 2.60 | 2.13 | 1.96 | 2.03 |
| 1:900 | 2.51 | 1.88 | 1.77 | 1.85 |
| 1:2700 | 1.45 | 1.21 | 1.15 | 1.34 |
| 1:8100 | 0.50 | 0.40 | 0,46 | 0.65 |
| 1:24300 | 0.18 | 0.17 | 0.22 | 0.24 |
| 1:72900 | 0.08 | 0.08 | 0.09 | 0.10 |
| 1:218700 | 0.06 | 0.07 | 0.05 | 0.06 |
| 1:656100 | 0.06 | 0.06 | 0.05 | 0.05 |
| 1:1968300 | 0.06 | 0.06 | 0.05 | 0.05 |
| 1:5904900 | 0.07 | 0.07 | 0.05 | 0.05 |
| Blank control | 0.07 | 0.07 | 0.06 | 0.07 |

3.2 Library construction

A total of 100 mL peripheral blood was collected from the llamas after the seventh immunization, and PBMC was isolated with a lymphocyte isolation medium; total RNA was extracted with an RNAiso Plus reagent (Takara, Cat. No. 9108/9109), the extracted RNA was reversely transcribed into cDNA with a PrimeScript™ II 1st Strand cDN A Synthesis Kit (Takara, Cat. No. 6210A). Nested PCR was applied to amplify the nucleic acid fragment encoding a heavy chain antibody variable region:

The first round of PCR:
forward primer:
(SEQ ID NO: 6)
CTTGGTGGTCCTGGCTGC;
reverse primer:
(SEQ ID NO: 7)
GGTACGTGCTGTTGAACTGTTCC.
The second round of PCR:
products from the first round of PCR were used as
templates,
forward primer (SEQ ID NO: 8):
CATGCCATGACTGTGGCCCAGGCGGCCCAGKTGCAGCTCGTGGAGTC;
reverse primer-1 (SEQ ID NO: 9):
CATGCCATGACTCGCGGCCGGCCTGGCCATGGGGGTCTTCGCTGTGGTGC
G;
reverse primer-2 (SEQ ID NO: 10):
CATGCCATGACTCGCGGCCGGCCTGGCCGTCTTGTGGTTTTGGTGTCTTG
GG.

The nucleic acid fragment of the target single domain antibody was recovered and cloned into the phage display vector pcomb3XSS (purchased from Sichuan NB Biolab Co., Ltd.) using the restriction endonuclease SfiI (NEB, Cat. No. R0123S). The product was then electrotransformed into E. coli electroporation competent cells TG1 (purchased from Sichuan NB Biolab Co., Ltd.), and a single domain antibody phage display library against CD33 was constructed and assayed. The size of the reservoir was calculated to be 3.4×10⁹ by gradient dilution plating. To check the library for insertion rate, 48 clones were randomly selected for colony PCR. The results show that the insertion rate is up to 100%.

### 3.3 Panning of Single Domain Antibodies Against CD33

A plate was coated with a human CD33-Llama-Fc fusion protein (ACROBiosystems, Cat. No. CD3-H5259) with 0.5 μg/well, and placed overnight at 4° C.; the plate was blocked with 3% BSA-PBS for 1 h at 37° C. on the next day, and then 100 μL of the phage display library was added; after incubation for 1 h at 37° C., the plate was washed 6 times with PBST and washed twice with PBS, so as to wash out unbound phages. Finally, 100 μL of Gly-HCl eluent was added to elute the phages specifically binding to CD33, thereby enriching positive clones.

### 3.4 Screening of Specific Single Positive Clones Using Phage Enzyme-Linked Immunosorbent Assay

The CD33-binding positive phages obtained after panning were infected with blank E. coli and plated. 96 single colonies were then selected, amplified and cultured, separately. The plates were coated with the CD33-his protein at 4° C., and incubated overnight. The phage cultural supernatant was added, and then the plates were incubated for 1 h at 37° C. A TMB color-developing solution was added for color developing after washing the plates, and optical density was measured at a wavelength of 450 nm. CD33-his positive clones were selected and sequenced. The sequencing results were analyzed using MOE software and a phylogenetic tree was constructed according to the amino acid sequences of VHH-encoded protein, the sequences with closer distance on the phylogenetic tree were eliminated according to sequence similarity, and then 18 clones were obtained by screening, the CDRs of the sequences of the clones were analyzed separately using KABAT, Chothia, or IMGT software. The corresponding sequence information is shown in Table 10 below. The production and identification of single domain antibodies were then carried out.

TABLE 10
Sequence information of the VhH single domain antibodies obtained by
screening

| Antibody name | Sequence No. | Amino acid sequence |
|---|---|---|
| S006-NB146-1 | SEQ ID NO: 11 | EVQVVESGGGLVQAGGSLTLSCAASGNIENIQVMGWYRQAP |
| | | GSQRALVAQINLSGLESTNYLDSVKERFTISRDYTKNAVYLRM |
| | | NRLEPEDTAVYYCNARSYYGQYDYWGQGTQVTVSS |
| S006-NB146-3 | SEQ ID NO: 12 | EVQVVESGGGLVQSGGSLRLSCAASGAIFTSNAMDYAMGWY |
| | | RQVSGKQREWVATITSDDDTNYGDSAKGRFTISRDSAKNTAY |
| | | LQMNSLKPEDTAVYYCFANVRSDRSTLYRNYWGQGTQVTVS |
| | | S |
| S006-NB146-17 | SEQ ID NO: 13 | QVQLVESGGGLVQSGGSLRLSCAASGSIFTSDDMDYAMGWY |
| | | RQASGKQREWVATITSDEDTNYGDSVKGRFTISRDSAKNTAF |
| | | LQMNTLKPEDTAVYYCFANVRSDRSSLYRNYWGQGTQVTVS |
| | | S |
| S006-NB146-20 | SEQ ID NO: 14 | EVQLVESGGGSVQAGGSLRLSCGTPQSWFRLSSYVMNWYRQ |
| | | APGKRRELVAEITQDDTTNYTPSVKGRFAISRDNANNTVHLQ |
| | | MNSLKPEDTAVYYCNTIQIWGQGTQVTVSS |
| S006-NB146-39 | SEQ ID NO: 15 | EVQLVESGGGLVQPGESLRLSCTAPGIILDYYPIGWFRQAPGK |
| | | EREGVAFITNSDDSTIYTNYADSVKGRFTISREKNTVYLQMNS |
| | | LKPEDTALYYCAARSTYIVGRNENALNSSDYDYWGQGTQVT |
| | | VSS |
| S006-NB146-40 | SEQ ID NO: 16 | QVQLVESGGGLVQSGGSLRLSCAASGSIFTSNDMDYAMGWY |
| | | RQVSGKQREWVATITSDEDINYGDSVKGRFTISRDSAKNTAY |
| | | LQMNSLKPEDTAVYYCFANLRSDRSTLYRNYWGQGTQVTVS |
| | | S |
| S006-NB146-46 | SEQ ID NO: 17 | QLQLVESGGGLVQPGGSLRLSCTAPGIVLDYYPIGWFRQAPG |
| | | KEREGVSFITNSDDSTIYTNYADSVKGRFTISREKNTVYLQMN |
| | | SLKPEDTAVYYCAARSTYIVGRNENALNASDYDYWGQGTQV |
| | | TVSS |
| S006-NB146-60 | SEQ ID NO: 18 | EVQLVESGGGLVQAGGSLRLSCVASRSTSSLSLMSWHRQAPG |
| | | EQRELVASITSGGKANYVDSVKDRFIMTRDDAKNTVYLQMN |
| | | SLKPEDTAVYYCHAYDRAGTASGFGAAWGQGTQVTVSS |
| S006-NB146-132 | SEQ ID NO: 19 | QVQLVESGGGLVQAGGSLRLSCAASGGTFVINAAWYHQAPG |
| | | KQRELVANISYGGSTNYADRVAGRFAISRDNAKNTVYLQMNS |
| | | LKPEDTGVYYCRLDPGLALNNRPLYYWGQGTQVTVSS |
| S006-NB147-225 | SEQ ID NO: 20 | EVQVVESGGGSVQPGGSLRLSCVASGFTFSNYAMSWVRQTP |
| | | KKGLEWVAEINSGGDNTYYAYFVKGRFTISRDNAKNTLYLQ |
| | | MNSLKPEDTGTYYCAREEENAGTILQGQGTQVTVSS |
| S006-NB146-16 | SEQ ID NO: 21 | EVQLVESGGGLVPAGGSLRLSCAASGSIANIQVMGWYRQAPG |
| | | SQRALVAQITVTGDRATNYADSVKGRFTISRDNAKNAVYLSIN |
| | | PLKSEDTGVYYCNARSYYGQHNYWGQGTQVTVSS |
| S006-NB147-25 | SEQ ID NO: 22 | QLQLVESGGGLVQAGGSLRLSCKASGVTFSINALGWYRQAPG |
| | | KQREMVAVISSGGSTNYVDSVKGRFTISRDNAKNTVYLQMNS |
| | | LKPEDTAVYYCNADVVGSSWSPEYDYWGQGTQVTVST |
| S006-NB146-69 | SEQ ID NO: 23 | EVQVVESGGGLVQAGGSLSLSCAASGSIFTINIMGWYRQAPG |
| | | KQRELVAQINRVGDTNYPNSMAERFTISRDNAKNTMYLQMN |
| | | TLKPEDTAVYYCNADIVESGRYSGKDYWGKGTLVTVSL |
| S006-NB146-139 | SEQ ID NO: 24 | QLQLVESGGGLVQPGGSLRLSCAAPGIILDYYPIGWFRQAPGK |
| | | EREGVSYITSSDDSTVYTYYTDSVKGRFTISRGKNTVYLQMN |
| | | SLKPEDTAVYYCAARSTYIVGRNYHTLNSSDFDYWGQGTQV |
| | | TVSS |
| S006-NB146-150 | SEQ ID NO: 25 | QLQLVESGGGLVQAGGSLRLSCAASGNSLGVYNLGWYRQAP |
| | | GEQRELVATINSRGDTNYVNSVAGRFTISRVNARNTENTVYLQ |
| | | MNRLQPEDTAVYYCIAQRGWGREYDIWGPGTQVTVSS |
| S006-NB146-173 | SEQ ID NO: 26 | QVQLVESGGGLVQAGGSLRLSCAASGSTLSLHSMGWYRQAA |
| | | GRQRELVAGISRDGDSNYLSSLAGRFFISRDGTKNTVYLQMTS |
| | | LKPEDTAVYYCNARTSPWSAALWGQGTQVTVSS |
| S006-NB146-181 | SEQ ID NO: 27 | QVQLVESGGGLVQAGGSLRLSCAASGSIFSMGWYRQAPGKQ |
| | | RELVAAIANGGTTTNYANSVKDRFTISRDNAKNTVYLQMNSL |
| | | RPEDTAVYYCYAKRSWDSSSYWGQGTQVTVSS |
| S006-NB147-220 | SEQ ID NO: 28 | QLQLVESGGGMVQPGGSLRLSCAASGFTLDYYAIGWFRQAP |
| | | GKEREGVSCINSGDSSTYYADSVKGRFTISRDNAENTVYLQM |
| | | NSLKPEDTAVYYCATVDTLIVAGNLEECRYDMDYWGKGTLV |
| | | TVSS |

KABAT Analysis

| | | | |
|---|---|---|---|
| Antibody name | CD1 | CDR2 | CDR3 |
| Sequence No. | Sequence No. | Sequence No. | Sequence No. |

| S006-NB146-1 | IQVMG | QINLSGLESTNYLDSVKE | RSYYGQYDY |
|---|---|---|---|
| (SEQ ID NO: 11) | (SEQ ID NO: 29) | (SEQ ID NO: 30) | (SEQ ID NO: 31) |
| S006-NB146-3 | SNAMDYAMG | TITSDDDTNYGDSAKG | NVRSDRSTLYRNY |
| (SEQ ID NO: 12) | (SEQ ID NO: 32) | (SEQ ID NO: 33) | (SEQ ID NO: 34) |
| S006-NB146-17 | SDDMDYAMG | TITSDEDINYGDSVKG | NVRSDRSSLYRNY |
| (SEQ ID NO: 13) | (SEQ ID NO: 35) | (SEQ ID NO: 36) | (SEQ ID NO: 37) |
| S006-NB146-20 | LSSYVMN | EITQDDTTNYTPSVKG | IQI |
| (SEQ ID NO: 14) | (SEQ ID NO: 38) | (SEQ ID NO: 39) | (SEQ ID NO: 40) |
| Antibody name | Sequence No. | Amino acid sequence | |
| S006-NB146-39 | YYPIG | TITNSDDSTIYTNYADSVKG | RSTYIVGRNENALNSSDYDY |
| (SEQ ID NO: 15) | (SEQ ID NO: 41) | (SEQ ID NO: 42) | (SEQ ID NO: 43) |
| S006-NB146-40 | SNDMDYAMG | TITSDEDINYGDSVKG | NLRSDRSTLYRNY |
| (SEQ ID NO: 16) | (SEQ ID NO: 44) | (SEQ ID NO: 45) | (SEQ ID NO: 46) |
| S006-NB146-46 | YYPIG | FITNSDDSTIYTNYADSVKG | RSTYIVGRNENALNASDYDY |
| (SEQ ID NO: 17) | (SEQ ID NO: 47) | (SEQ ID NO: 48) | (SEQ ID NO: 49) |
| S006-NB146-60 | LSLMS | SITSGGKANYVDSVKD | YDRAGTASGFGAA |
| (SEQ ID NO: 18) | (SEQ ID NO: 50) | (SEQ ID NO: 51) | (SEQ ID NO: 52) |
| S006-NB146-132 | INAA | NISYGGSTNYADRVAG | DPGLALNNRPLYY |
| (SEQ ID NO: 19) | (SEQ ID NO: 53) | (SEQ ID NO: 54) | (SEQ ID NO: 55) |
| S006-NB147-225 | NYAMS | EINSGGDNTYYAYFVKG | EEENAGTIL |
| (SEQ ID NO: 20) | (SEQ ID NO: 56) | (SEQ ID NO: 57) | (SEQ ID NO: 58) |
| S006-NB146-16 | IQVMG | QITVTGDRATNYADSVKG | RSYYGQHNY |
| (SEQ ID NO: 21) | (SEQ ID NO: 59) | (SEQ ID NO: 60) | (SEQ ID NO: 61) |
| S006-NB147-25 | INALG | VISSGGSTNYVDSVKG | DVVGSSWSPEYDY |
| (SEQ ID NO: 22) | (SEQ ID NO: 62) | (SEQ ID NO: 63) | (SEQ ID NO: 64) |
| S006-NB146-69 | INIMG | QINRVGDTNYPNSMAE | DIVESGRYSGKDY |
| (SEQ ID NO: 23) | (SEQ ID NO: 65) | (SEQ ID NO: 66) | (SEQ ID NO: 67) |
| S006-NB146-139 | YYPIG | YITSSDDSTVYTYYTDSVKG | RSTYIVGRNYHTLNSSDFDY |
| (SEQ ID NO: 24) | (SEQ ID NO: 68) | (SEQ ID NO: 69) | (SEQ ID NO: 70) |
| S006-NB146-150 | VYNLG | TINSRGDTNYVNSVAG | QRGWGREYDI |
| (SEQ ID NO: 25) | (SEQ ID NO: 71) | (SEQ ID NO: 72) | (SEQ ID NO: 73) |
| S006-NB146-173 | LHSMG | GISRDGDSNYLSSLAG | RTSPWSAAL |
| (SEQ ID NO: 26) | (SEQ ID NO: 74) | (SEQ ID NO: 75) | (SEQ ID NO: 76) |
| S006-NB146-181 | MG | AIANGGTTTNYANSVKD | KRSWDSSSY |
| (SEQ ID NO: 27) | (SEQ ID NO: 77) | (SEQ ID NO: 78) | (SEQ ID NO: 79) |
| S006-NB147-220 | YYAIG | CINSGDSSTYYADSVKG | VDTLIVAGNLEECRYDMDY |
| (SEQ ID NO: 28) | (SEQ ID NO: 80) | (SEQ ID NO: 81) | (SEQ ID NO: 82) |

Chothia Analysis

| Antibody name | CDR1 | CDR2 | CDR3 |
|---|---|---|---|
| Sequence No. | Sequence No. | Sequence No. | Sequence No. |
| S006-NB146-1 | GNIENIQ | NLSGLES | RSYYGQYDY |
| (SEQ ID NO: 11) | (SEQ ID NO: 83) | (SEQ ID NO: 84) | (SEQ ID NO: 85) |
| S006-NB146-3 | GAIFTSNAMDY | TSDDD | NVRSDRSTLYRNY |
| (SEQ ID NO: 12) | (SEQ ID NO: 86) | (SEQ ID NO: 87) | (SEQ ID NO: 88) |
| S006-NB146-17 | GSIFTSDDMDY | TSDED | NVRSDRSSLYRNY |
| (SEQ ID NO: 13) | (SEQ ID NO: 89) | (SEQ ID NO: 90) | (SEQ ID NO: 91) |
| Antibody name | Sequence No. | Amino acid sequence | |
| S006-NB146-20 | QSWFRLSSY | TQDDT | IQI |
| (SEQ ID NO: 14) | (SEQ ID NO: 92) | (SEQ ID NO: 93) | (SEQ ID NO: 94) |
| S006-NB146-39 | GIILDYY | TNSDDSTIY | RSTYIVGRNENALNSSDYDY |
| (SEQ ID NO: 15) | (SEQ ID NO: 95) | (SEQ ID NO: 96) | (SEQ ID NO: 97) |
| S006-NB146-40 | GSIFTSNDMDY | TSDED | NLRSDRSTLYRNY |
| (SEQ ID NO: 16) | (SEQ ID NO: 98) | (SEQ ID NO: 99) | (SEQ ID NO: 100) |
| S006-NB146-46 | GIVLDYY | TNSDDSTIY | RSTYIVGRNENALNASDYDY |
| (SEQ ID NO: 17) | (SEQ ID NO: 101) | (SEQ ID NO: 102) | (SEQ ID NO: 103) |
| S006-NB146-60 | RSTSSLS | TSGGK | YDRAGTASGFGAA |
| (SEQ ID NO: 18) | (SEQ ID NO: 104) | (SEQ ID NO: 105) | (SEQ ID NO: 106) |
| S006-NB146-132 | GGTFVI | SYGGS | DPGLALNNRPLYY |
| (SEQ ID NO: 19) | (SEQ ID NO: 107) | (SEQ ID NO: 108) | (SEQ ID NO: 109) |
| S006-NB147-225 | GFTFSNY | NSGGDN | EEENAGTIL |
| (SEQ ID NO: 20) | (SEQ ID NO: 110) | (SEQ ID NO: 111) | (SEQ ID NO: 112) |
| S006-NB146-16 | GSIANIQ | TVTGDRA | RSYYGQHNY |
| (SEQ ID NO: 21) | (SEQ ID NO: 113) | (SEQ ID NO: 114) | (SEQ ID NO: 115) |
| S006-NB147-25 | GVTFSIN | SSGGS | DVVGSSWSPEYDY |
| (SEQ ID NO: 22) | (SEQ ID NO: 116) | (SEQ ID NO: 117) | (SEQ ID NO: 118) |
| S006-NB146-69 | GSIFTIN | NRVGD | DIVESGRYSGKDY |
| (SEQ ID NO: 23) | (SEQ ID NO: 119) | (SEQ ID NO: 120) | (SEQ ID NO: 121) |
| S006-NB146-139 | GIILDYY | TSSDDSTVY | RSTYIVGRNYHTLNSSDFDY |
| (SEQ ID NO: 24) | (SEQ ID NO: 122) | (SEQ ID NO: 123) | (SEQ ID NO: 124) |
| S006-NB146-150 | GNSLGVY | NSRGD | QRGWGREYDI |
| (SEQ ID NO: 25) | (SEQ ID NO: 125) | (SEQ ID NO: 126) | (SEQ ID NO: 127) |
| S006-NB146-173 | GSTLSLH | SRDGD | RTSPWSAAL |
| (SEQ ID NO: 26) | (SEQ ID NO: 128) | (SEQ ID NO: 129) | (SEQ ID NO: 130) |
| S006-NB146-181 | GSIF | ANGGTT | KRSWDSSSY |
| (SEQ ID NO: 27) | (SEQ ID NO: 131) | (SEQ ID NO: 132) | (SEQ ID NO: 133) |
| S006-NB147-220 | GFTLDYY | NSGDSS | VDTLIVAGNLEECRYDMDY |
| (SEQ ID NO: 28) | (SEQ ID NO: 134) | (SEQ ID NO: 135) | (SEQ ID NO: 136) |

IMGT Analysis

| Antibody name | CDR1 | CDR2 | CDR3 |
|---|---|---|---|
| Sequence No. | Sequence No. | Sequence No. | Sequence No. |
| S006-NB146-1 | GNIENIQV | INLSGLEST | NARSYYGQYDY |
| (SEQ ID NO: 11) | (SEQ ID NO: 137) | (SEQ ID NO: 138) | (SEQ ID NO: 139) |
| S006-NB146-3 | GAIFTSNAMDYA | ITSDDDT | FANVRSDRSTLYRNY |
| Antibody name | Sequence No. | Amino acid sequence | |
| (SEQ ID NO: 12) | (SEQ ID NO: 140) | (SEQ ID NO: 141) | (SEQ ID NO: 142) |
| S006-NB146-17 | GSIFTSDDMDYA | ITSDEDT | FANVRSDRSSLYRNY |
| (SEQ ID NO: 13) | (SEQ ID NO: 143) | (SEQ ID NO: 144) | (SEQ ID NO: 145) |
| S006-NB146-20 | QSWFRLSSYV | ITQDDTT | NTIQI |
| (SEQ ID NO: 14) | (SEQ ID NO: 146) | (SEQ ID NO: 147) | (SEQ ID NO: 148) |
| S006-NB146-39 | GULDYYP | ITNSDDSTIYT | AARSTYIVGRNENALNSSDY |
| (SEQ ID NO: 15) | (SEQ ID NO: 149) | (SEQ ID NO: 150) | DY (SEQ ID NO: 151) |
| S006-NB146-40 | GSIFTSNDMDYA | ITSDEDT | FANLRSDRSTLYRNY |
| (SEQ ID NO: 16) | (SEQ ID NO: 152) | (SEQ ID NO: 153) | (SEQ ID NO: 154) |
| S006-NB146-46 | GIVLDYYP | ITNSDDSTIYT | AARSTYIVGRNENALNASDY |
| (SEQ ID NO: 17) | (SEQ ID NO: 155) | (SEQ ID NO: 156) | DY (SEQ ID NO: 157) |
| S006-NB146-60 | RSTSSLSL | ITSGGKA | HAYDRAGTASGFGAA |
| (SEQ ID NO: 18) | (SEQ ID NO: 158) | (SEQ ID NO: 159) | (SEQ ID NO: 160) |
| S006-NB146-132 | GGTFVIN | ISYGGST | RLDPGLALNNRPLYY |
| (SEQ ID NO: 19) | (SEQ ID NO: 161) | (SEQ ID NO: 162) | (SEQ ID NO: 163) |
| S006-NB147-225 | GFTFSNYA | INSGGDNT | AREEENAGTIL |
| (SEQ ID NO: 20) | (SEQ ID NO: 164) | (SEQ ID NO: 165) | (SEQ ID NO: 166) |
| S006-NB146-16 | GSIANIQV | ITVTGDRAT | NARSYYGQHNY |
| (SEQ ID NO: 21) | (SEQ ID NO: 167) | (SEQ ID NO: 168) | (SEQ ID NO: 169) |
| S006-NB147-25 | GVTFSINA | ISSGGST | NADVVGSSWSPEYDY |
| (SEQ ID NO: 22) | (SEQ ID NO: 170) | (SEQ ID NO: 171) | (SEQ ID NO: 172) |
| S006-NB146-69 | GSIFTINI | INRVGDT | NADIVESGRYSGKDY |
| (SEQ ID NO: 23) | (SEQ ID NO: 173) | (SEQ ID NO: 174) | (SEQ ID NO: 175) |
| S006-NB146-139 | GULDYYP | ITSSDDSTVYT | AARSTYIVGRNYHTLNSSDF |
| (SEQ ID NO: 24) | (SEQ ID NO: 176) | (SEQ ID NO: 177) | DY (SEQ ID NO: 178) |
| S006-NB146-150 | GNSLGVYN | INSRGDT | LAQRGWGREYDI |
| (SEQ ID NO: 25) | (SEQ ID NO: 179) | (SEQ ID NO: 180) | (SEQ ID NO: 181) |
| S006-NB146-173 | GSTLSLHS | ISRDGDS | NARTSPWSAAL |
| (SEQ ID NO: 26) | (SEQ ID NO: 182) | (SEQ ID NO: 183) | (SEQ ID NO: 184) |
| S006-NB146-181 | GSIFS | IANGGTTT | YAKRSWDSSSY |
| (SEQ ID NO: 27) | (SEQ ID NO: 185) | (SEQ ID NO: 186) | (SEQ ID NO: 187) |
| S006-NB147-220 | GFTLDYYA | INSGDSST | ATVDTLIVAGNLEECRYDMD |
| (SEQ ID NO: 28) | (SEQ ID NO: 188) | (SEQ ID NO: 189) | Y (SEQ ID NO: 190) |

### Example 4: Preparation of VHH Single Domain Antibodies

### 4.1 Expression and Purification of VHH Single Domain Antibodies

The VHH variable region sequences were recombined into the expression vector BI3.4-huIgG1 containing a signal peptide and human IgG1 Fc (the human IgG1 Fc sequence set forth in SEQ ID NO: 191, and the hinge region sequence set forth in SEQ ID NO: 192) by Biointron (Taizhou City) Biological Inc., and plasmids were prepared according to established standard molecular biology methods. See Sambrook, J., Fritsch, E. F., and Maniatis, T. (1989). Molecular Cloning: A Laboratory Manual, Second Edition (Plainview, New York: Cold Spring Harbor Laboratory Press). HEK293E cells (purchased from Suzhou Yiyan Biotech Co., Ltd.) were transiently transfected with the expression vectors according to PEI (purchased from Polysciences, Cat. No. 24765-1) instructions, cultured at 37° C. for 5 consecutive days using FreeStyle™ 293 (Thermofisher scientific, Cat. No. 12338018), and centrifuged to remove cell components, so as to obtain culture supernatants containing the VHH single domain antibodies. The culture supernatants were each loaded on a Protein A chromatography column (the Protein A packing AT Protein A Diamond and chromatography column BXK16/26 were purchased from Bestchrom, with Cat. Nos. of AA0273 and B-1620, respectively), washed with phosphate-buffered saline (PBS; pH 7.4), then washed with 20 mM PB, 1 M NaCl (pH 7.2), and finally subjected to elution with a citrate buffer at pH 3.4. An Fc-tagged antibody eluted from the Protein A chromatography column was collected, neutralized with 1/10 volumes of 1 M Tris at pH 8.0, and dialyzed with PBS at 4° C. overnight. The dialyzed protein was subjected to sterile filtration through a 0.22 μM filter membrane, subpackaged, and stored at −80° C.

Sequence of human IgG1 Fc (SEQ ID NO: 191):
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHE
ALHNHYTQKSLSLSPGK
Hinge region sequence (SEQ ID NO: 192):
EPKSADKTHTCPPCP

### Example 5: Identification of VHH Single Domain Antibodies

### 5.1 Assay on Binding of VHH Antibodies to CD33 Protein by Enzyme-Linked Immunosorbent Assay (ELISA)

In order to assay the binding activity of VHH-Fc to human CD33 protein, a human CD33-his protein (purchased from ACROBiosystems, Cat. No. CD3-H5226) was diluted with PBS to make a final concentration of 2 μg/mL, and then added into a 96-well ELISA plate at 100 μL/well. The plate was sealed with a plastic film and incubated at 4° C. overnight, washed twice with PBS the next day, and a blocking solution [PBS+2% (w/w) BSA] was added for blocking at room temperature for 2 h. The blocking solution was discarded, and VHH-Fc antibody or negative control antibody serially diluted from 100 nM was added at 50 μL/well. After incubation at 37° C. for 2 h, the plate was washed 3 times with PBS. A secondary antibody labeled with HRP (horseradish peroxidase) (purchased from Sigma, Cat. No. A0170) was added. After incubation for 1 h at 37° C., the plate was washed 5 times with PBS. A TMB substrate was added at 50 μL/well. After incubation at room temperature for 10 min, a stop solution (1.0 N HCl) was added at 50 μL/well. The OD450 nm values were read on an ELISA plate reader (Multimode Plate Reader, EnSight, purchased from Perkin Elmer). The assay results for the binding of the VHH-Fc to hCD33-his by ELISA are shown in FIGS. 7A and 7B and Tables 11 and 12. Tables 11 and 12 show that all the purified antibodies can bind to the human CD33 protein at the ELISA level. The IgG control was hIgG1, and the data in the table are OD450 nm values.

TABLE 11
Assay results for binding reactions of VHH-Fc
antibodies with human CD33 protein by ELISA

| OD450 | | | | | | | | |
|---|---|---|---|---|---|---|---|---|
| Antibody | Antibody concentration (nM) | | | | | | | |
| name | 100 | 10 | 1 | 0.1 | 0.01 | 0.001 | 0.0001 | blank |
| S006-NB146-1 | 3.20 | 2.88 | 2.25 | 0.29 | 0.10 | 0.08 | 0.08 | 0.08 |
| S006-NB146-3 | 2.95 | 2.81 | 1.58 | 0.26 | 0.07 | 0.06 | 0.06 | 0.08 |
| S006-NB146-17 | 3.48 | 3.02 | 1.77 | 0.24 | 0.06 | 0.06 | 0.06 | 0.06 |
| S006-NB146-20 | 2.75 | 2.82 | 1.55 | 0.47 | 0.09 | 0.07 | 0.06 | 0.07 |
| S006-NB146-39 | 2.97 | 2.81 | 1.81 | 0.14 | 0.06 | 0.06 | 0.05 | 0.06 |
| S006-NB146-40 | 2.90 | 2.80 | 2.46 | 0.27 | 0.07 | 0.05 | 0.05 | 0.06 |
| S006-NB146-46 | 2.85 | 2.91 | 1.92 | 0.19 | 0.07 | 0.06 | 0.06 | 0.06 |
| S006-NB146-60 | N/A | 2.11 | 2.04 | 0.48 | 0.15 | 0.07 | 0.06 | 0.06 |
| S006-NB146-132 | 3.24 | 2.78 | 1.97 | 0.40 | 0.11 | 0.08 | 0.07 | 0.06 |
| S006-NB147-225 | 2.94 | 2.44 | 1.77 | 0.36 | 0.09 | 0.06 | 0.05 | 0.06 |
| C33B904 | 2.96 | 2.60 | 1.39 | 0.28 | 0.08 | 0.06 | 0.04 | 0.07 |
| SGN-33 | 1.35 | 1.56 | 0.71 | 0.39 | 0.08 | 0.06 | 0.05 | 0.06 |
| hIgG1 | 0.05 | 0.05 | 0.05 | 0.05 | 0.06 | 0.06 | 0.05 | 0.06 |

TABLE 12
Assay results for binding reactions of VHH-Fc
antibodies with human CD33 protein by ELISA

| OD450 | | | | | | | | |
|---|---|---|---|---|---|---|---|---|
| Antibody | Antibody concentration (nM) | | | | | | | |
| name | 100 | 10 | 1 | 0.1 | 0.01 | 0.001 | 0.0001 | blank |
| S006-NB146-16 | 1.99 | 2.05 | 1.79 | 0.92 | 0.32 | 0.08 | 0.05 | 0.04 |
| S006-NB147-25 | 2.35 | 2.18 | 1.87 | 0.73 | 0.19 | 0.06 | 0.05 | 0.04 |
| S006-NB146-69 | 1.84 | 1.92 | 2.06 | 0.50 | 0.30 | 0.09 | 0.05 | 0.04 |
| S006-NB146-139 | 1.95 | 2.16 | 1.39 | 0.57 | 0.14 | 0.06 | 0.04 | 0.04 |
| S006-NB146-150 | 1.96 | 1.93 | 1.72 | 0.87 | 0.23 | 0.06 | 0.05 | 0.04 |
| S006-NB146-173 | 2.25 | 2.21 | 2.13 | 0.91 | 0.43 | 0.09 | 0.06 | 0.04 |
| S006-NB146-181 | 1.91 | 1.93 | 1.65 | 0.58 | 0.17 | 0.06 | 0.04 | 0.04 |
| S006-NB147-220 | 2.17 | 1.92 | 1.86 | 0.46 | 0.17 | 0.06 | 0.04 | 0.04 |
| C33B904 | 2.61 | 2.55 | 1.92 | 0.53 | 0.14 | 0.05 | 0.04 | 0.04 |
| SGN-33 | 1.30 | 1.56 | 1.48 | 0.74 | 0.26 | 0.06 | 0.04 | 0.04 |
| hIgG1 | 0.06 | 0.04 | 0.03 | 0.03 | 0.04 | 0.04 | 0.04 | 0.04 |

### 5.2 Assay on Binding of Antibodies to Different CD33-Expressing Cells by Flow Cytometry Assay (FACS)

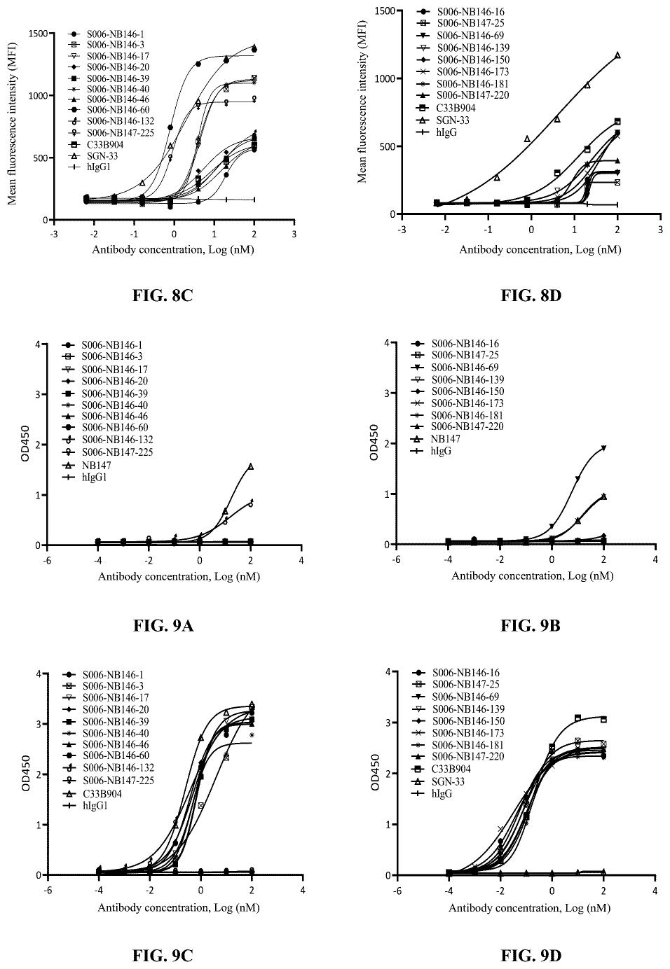

The desired cells were expanded to a logarithmic growth phase in a T-75 cell culture flask. For adherent cells CHO-K1, the culture medium was removed by pipetting, and the cells were washed twice with a PBS buffer, digested with trypsin, and washed twice with a PBS buffer again after the digestion was stopped. For suspension cells U937, the medium supernatant was discarded by direct centrifugation, and the cell pellet was washed twice with PBS, and then incubated with a buffer containing human Fc Block (purchased from BD, Cat. No. 564220) for 15 min. After the cells obtained in the last step were subjected to cell counting, the cell pellet was resuspended to 2×10⁶ cells/mL with a blocking solution [PBS+2% (w/w) BSA] and added to a 96-well FACS reaction plate at 50 μL/well. The VHH-Fc antibody (a sample to be tested) was added at 50 μL/well, and the plate was incubated on ice for 2 h. After the plate was centrifuged and washed 3 times with a PBS buffer, an Alexa Flour 488-labeled secondary antibody (purchased from Invitrogen, Cat. No. A-11013) was added at 50 μL/well, and the plate was incubated on ice for 1 h. After the plate was centrifuged and washed 5 times with a PBS buffer, the results were assayed and analyzed by FACS (FACS Canto™, purchased from BD Biosciences), so as to obtain the mean fluorescence intensity (MFI) of cells. Then, analysis was performed by software (GraphPad Prism8), data were fitted, and EC50 values were calculated. The analysis results are shown in Tables 13 and 14 and FIGS. 8A-8D, which show that all the VHH antibodies can bind to the human CD33 protein on the surface of U937 cells and CHO-K1-hCD33-B8 cells (FIGS. 8A-8D), but not to negative cells.

TABLE 13
Assay results for binding reactions of VHH-Fc antibodies
with U937 and CHO-K1-human CD33 cells by FACS

| | U937 | | CHO-K1-human CD33 | |
|---|---|---|---|---|
| | Maximum | | Maximum | |
| | mean | | mean | |
| | fluorescence | | fluorescence | |
| | intensity | EC50 | intensity | EC50 |
| Antibody name | (Max MFI) | (nM) | (Max MFI) | (nM) |
| S006-NB146-1 | 563 | 19.31 | 11417 | 177.40 |
| S006-NB146-3 | 1140 | 4.09 | 6418 | 1.04 |
| S006-NB146-17 | 1141 | 4.38 | 6342 | 1.09 |
| S006-NB146-20 | 668 | 5.32 | 20452 | 9.75 |
| S006-NB146-39 | 650 | 11.30 | 16264 | 44.44 |
| S006-NB146-40 | 1102 | 3.82 | 6432 | 1.09 |
| S006-NB146-46 | 589 | 14.73 | 16529 | 38.00 |
| S006-NB146-60 | 1367 | 0.80 | 23015 | 1.29 |
| S006-NB146-132 | 694 | 15.39 | 15236 | 48.53 |
| S006-NB147-225 | 967 | 0.92 | 15348 | 1.29 |
| C33B904 | 599 | 8.41 | 15004 | 12.52 |
| SGN-33 | 1387 | 1.99 | 9916 | 0.39 |
| hIgG1 | 162 | Negative | 389 | Negative |

TABLE 14
Assay results for binding reactions of VHH-Fc antibodies
with U937 and CHO-K1-human CD33 cells by FACS

| | U937 | | CHO-K1-human CD33 | |
|---|---|---|---|---|
| | Maximum | | Maximum | |
| | mean | | mean | |
| | fluorescence | | fluorescence | |
| | intensity | EC50 | intensity | EC50 |
| Antibody name | (Max MFI) | (nM) | (Max MFI) | (nM) |
| S006-NB146-16 | 598 | 40.35 | 11491 | 52.91 |
| S006-NB147-25 | 234 | ~18.51 | 9778 | 49.93 |
| S006-NB146-69 | 301 | ~22.66 | 5951 | 96.54 |
| S006-NB146-139 | 599 | 25.56 | 11941 | 14.60 |
| S006-NB146-150 | 307 | ~18.66 | 9075 | 76.77 |
| S006-NB146-173 | 575 | 30.92 | 11437 | 28.07 |
| S006-NB146-181 | 313 | ~20.95 | 8490 | 107.90 |
| S006-NB147-220 | 394 | 8.60 | 10047 | 52.01 |
| C33B904 | 682 | 12.63 | 12445 | 4.53 |
| SGN-33 | 1172 | 4.05 | 6923 | 0.16 |
| hIgG1 | 84 | Negative | 140 | Negative |

### Example 6: Assay on Species Cross-Binding Activity of VHH-Fc Antibodies

### 6.1 Assay on Binding of VHH-Fc Antibodies to CD33 Proteins of Different Species by ELISA

To assay the species cross-activity of the VHH-Fc antibodies, ELISA plates were coated with commercial murine CD33 (Sino Biological, Cat. No. 90303-C08H) and monkey CD33 (Sino Biological, Cat. No. 50712-M08H), respectively, and ELISA assays were performed according to the method in Example 5.1. The assay results for the binding of the VHH-Fc antibodies to murine CD33 by ELISA are shown in FIGS. 9A-9B and Tables 15 and 16, and the assay results for the binding of the VHH-Fc antibodies to monkey CD33 by ELISA are shown in FIGS. 9C and 9D and Tables 17 and 18. The IgG control was hIgG1, and the data in the table are OD450 nm values. Only S006-NB146-69 could bind well to the murine CD33 protein, and S006-NB146-132 and S006-NB146-173 bound weakly to the murine CD33 protein. Except for S006-NB146-60 and S006-NB147-225, VHH-Fc could bind to the monkey CD33 protein; SGN-33 did not bind to the monkey CD33 protein as shown in FIG. 9D.

TABLE 15
Assay results for binding reactions of VHH-Fc
antibodies with murine CD33 protein by ELISA

| OD450 | | | | | | | | |
|---|---|---|---|---|---|---|---|---|
| Antibody | Antibody concentration (nM) | | | | | | | |
| name | 100 | 10 | 1 | 0.1 | 0.01 | 0.001 | 0.0001 | blank |
| S006-NB146-1 | 0.08 | 0.06 | 0.06 | 0.06 | 0.07 | 0.07 | 0.07 | 0.07 |
| S006-NB146-3 | 0.07 | 0.07 | 0.06 | 0.06 | 0.06 | 0.06 | 0.06 | 0.06 |
| S006-NB146-17 | 0.07 | 0.06 | 0.06 | 0.07 | 0.06 | 0.06 | 0.06 | 0.08 |
| S006-NB146-20 | 0.06 | 0.06 | 0.06 | 0.06 | 0.06 | 0.06 | 0.06 | 0.12 |
| S006-NB146-39 | 0.06 | 0.06 | 0.06 | 0.06 | 0.06 | 0.06 | 0.06 | 0.06 |
| S006-NB146-40 | 0.06 | 0.06 | 0.06 | 0.06 | 0.06 | 0.06 | 0.06 | 0.07 |
| S006-NB146-46 | 0.06 | 0.06 | 0.06 | 0.06 | 0.06 | 0.06 | 0.06 | 0.07 |
| S006-NB146-60 | 0.07 | 0.07 | 0.06 | 0.06 | 0.06 | 0.07 | 0.06 | 0.06 |
| S006-NB146-132 | 0.84 | 0.49 | 0.17 | 0.14 | 0.06 | 0.06 | 0.06 | 0.06 |
| S006-NB147-225 | 0.06 | 0.06 | 0.06 | 0.05 | 0.11 | 0.06 | 0.06 | 0.06 |
| NB147* | 1.57 | 0.68 | 0.13 | 0.06 | 0.05 | 0.05 | 0.05 | 0.05 |
| hIgG1 | 0.06 | 0.06 | 0.06 | 0.06 | 0.06 | 0.06 | 0.05 | 0.06 |

TABLE 16
Assay results for binding reactions of VHH-Fc
antibodies with murine CD33 protein by ELISA

| OD450 | | | | | | | | |
|---|---|---|---|---|---|---|---|---|
| Antibody | Antibody concentration (nM) | | | | | | | |
| name | 100 | 10 | 1 | 0.1 | 0.01 | 0.001 | 0.0001 | blank |
| S006-NB146-16 | 0.07 | 0.06 | 0.05 | 0.05 | 0.06 | 0.10 | 0.06 | 0.05 |
| S006-NB147-25 | 0.07 | 0.06 | 0.05 | 0.05 | 0.05 | 0.06 | 0.06 | 0.05 |
| S006-NB146-69 | 1.90 | 1.29 | 0.35 | 0.10 | 0.07 | 0.06 | 0.06 | 0.06 |
| S006-NB146-139 | 0.07 | 0.06 | 0.09 | 0.06 | 0.06 | 0.06 | 0.06 | 0.05 |
| S006-NB146-150 | 0.17 | 0.08 | 0.07 | 0.06 | 0.06 | 0.06 | 0.06 | 0.06 |
| S006-NB146-173 | 0.94 | 0.46 | 0.13 | 0.07 | 0.06 | 0.06 | 0.06 | 0.06 |
| S006-NB146-181 | 0.07 | 0.06 | 0.06 | 0.05 | 0.05 | 0.06 | 0.06 | 0.05 |
| S006-NB147-220 | 0.07 | 0.06 | 0.06 | 0.06 | 0.06 | 0.06 | 0.06 | 0.05 |
| NB147* | 0.95 | 0.47 | 0.11 | 0.04 | 0.04 | 0.04 | 0.04 | 0.04 |
| hIgG1 | 0.08 | 0.07 | 0.06 | 0.06 | 0.06 | 0.06 | 0.06 | 0.06 |

*NB147 llama serum was subjected to a 10-fold gradient dilution starting at a 1:100 dilution ratio.

TABLE 17
Assay results for binding reactions of VHIHI-Fc
antibodies with monkey CD33 protein by ELISA

| OD450 | | | | | | | | |
|---|---|---|---|---|---|---|---|---|
| Antibody | Antibody concentration (nM) | | | | | | | |
| name | 100 | 10 | 1 | 0.1 | 0.01 | 0.001 | 0.0001 | blank |
| S006-NB146-1 | 3.22 | 2.78 | 2.15 | 0.63 | 0.16 | 0.10 | 0.10 | 0.09 |
| S006-NB146-3 | 3.33 | 2.33 | 1.38 | 0.33 | 0.11 | 0.06 | 0.06 | 0.07 |
| S006-NB146-17 | 3.28 | 3.06 | 2.05 | 0.43 | 0.07 | 0.06 | 0.05 | 0.06 |
| S006-NB146-20 | 3.05 | 2.92 | 2.23 | 0.66 | 0.11 | 0.07 | 0.06 | 0.06 |
| S006-NB146-39 | 3.09 | 2.89 | 1.95 | 0.21 | 0.07 | 0.06 | 0.06 | 0.06 |
| S006-NB146-40 | 2.78 | 2.40 | 2.00 | 0.34 | 0.06 | 0.06 | 0.05 | 0.06 |
| S006-NB146-46 | 3.00 | 2.93 | 1.98 | 0.25 | 0.08 | 0.06 | 0.06 | 0.06 |
| S006-NB146-60 | 0.06 | 0.06 | 0.05 | 0.05 | 0.05 | 0.05 | 0.05 | 0.06 |
| S006-NB146-132 | 3.27 | 2.93 | 2.16 | 1.09 | 0.27 | 0.14 | 0.11 | 0.06 |
| S006-NB147-225 | 0.07 | 0.06 | 0.05 | 0.06 | 0.06 | 0.06 | 0.05 | 0.06 |
| C33B904 | 3.40 | 3.23 | 2.73 | 0.99 | 0.15 | 0.06 | 0.06 | 0.10 |
| hIgG1 | 0.06 | 0.06 | 0.05 | 0.05 | 0.05 | 0.07 | 0.05 | 0.06 |

TABLE 18
Assay results for binding reactions of VHH-Fc
antibodies with monkey CD33 protein by ELISA

| OD450 | | | | | | | | |
|---|---|---|---|---|---|---|---|---|
| Antibody | Antibody concentration (nM) | | | | | | | |
| name | 100 | 10 | 1 | 0.1 | 0.01 | 0.001 | 0.0001 | blank |
| S006-NB146-16 | 2.34 | 2.42 | 2.26 | 1.53 | 0.67 | 0.13 | 0.06 | 0.04 |
| S006-NB147-25 | 2.58 | 2.64 | 2.44 | 1.39 | 0.47 | 0.10 | 0.06 | 0.05 |
| S006-NB146-69 | 2.33 | 2.45 | 2.21 | 1.18 | 0.41 | 0.09 | 0.05 | 0.05 |
| S006-NB146-139 | 2.46 | 2.52 | 2.27 | 1.39 | 0.45 | 0.09 | 0.05 | 0.04 |
| S006-NB146-150 | 2.37 | 2.44 | 2.41 | 1.50 | 0.57 | 0.10 | 0.05 | 0.04 |
| S006-NB146-173 | 2.49 | 2.50 | 2.15 | 1.60 | 0.91 | 0.17 | 0.07 | 0.04 |
| S006-NB146-181 | 2.31 | 2.33 | 2.21 | 1.02 | 0.26 | 0.07 | 0.05 | 0.04 |
| S006-NB147-220 | 2.47 | 2.46 | 2.23 | 1.19 | 0.34 | 0.08 | 0.05 | 0.04 |
| C33B904 | 3.06 | 3.09 | 2.52 | 1.15 | 0.29 | 0.07 | 0.04 | 0.04 |
| hIgG1 | 0.08 | 0.05 | 0.04 | 0.05 | 0.04 | 0.04 | 0.06 | 0.05 |

6.2 Assay on Binding Reactions of VHH-Fc Antibodies with Monkey CD33 Recombination Cell Line by FACS

HEK293T-monkey CD33 cell line was subjected to the FACS assay and data analysis according to the method in Example 5.2. The analysis results are shown in Tables 19 and 20 and FIGS. 10A-10B, which show that, except for S006-NB147-60 and S006-NB147-225, the remaining VHH-Fc antibodies had binding activity with HEK293T-monkey CD33 cells, but had no binding activity with HEK293T cells.

TABLE 19
Assay results for binding reactions of VHH antibodies
with HEK293T-monkey CD33 cell line by FACS

| | HEK293T-monkey CD33 | |
|---|---|---|
| | Maximum mean | |
| | fluorescence | |
| | intensity | |
| Antibody name | (Max MFI) | EC50 (nM) |
| S006-NB146-1 | 1476 | 237.10 |
| S006-NB146-3 | 2310 | 4.71 |
| S006-NB146-17 | 2293 | 4.02 |
| S006-NB146-20 | 2157 | 2.79 |
| S006-NB146-39 | 2179 | 5.92 |
| S006-NB146-40 | 2257 | 4.48 |
| S006-NB146-46 | 2152 | 6.13 |
| S006-NB146-60 | 104 | Negative |
| S006-NB146-132 | 2127 | 7.61 |
| S006-NB147-225 | 86 | Negative |
| C33B904 | 3592 | 5.70 |
| hIgG1 | 322 | Negative |

TABLE 20
Assay results for binding reactions of VHH antibodies
with HEK293T-monkey CD33 cell line by FACS

| | HEK293T-monkey CD33 | |
|---|---|---|
| | Maximum mean | |
| | fluorescence | |
| | intensity | |
| Antibody name | (Max MFI) | EC50 (nM) |
| S006-NB146-16 | 2800 | 120.60 |
| S006-NB147-25 | 4893 | 31.16 |
| S006-NB146-69 | 3455 | 66.10 |
| S006-NB146-139 | 3949 | 24.97 |
| S006-NB146-150 | 3313 | 45.15 |
| S006-NB146-173 | 5076 | 39.39 |
| S006-NB146-181 | 2905 | 121.30 |
| S006-NB147-220 | 6187 | 10.15 |
| C33B904 | 7877 | 4.80 |
| hIgG1 | 107 | Negative |

### Example 7: Assay on Affinity of CD33 Antibodies

### 7.1 Assay on Affinity of VHH-Fc Antibodies for Human CD33-his Protein

The human CD33 VHH-Fc antibodies were captured using a Protein A chip (GE Healthcare; 29-127-558). The sample and running buffer was HBS-EP+ (10 mM HEPES, 150 mM NaCl, 3 mM EDTA, 0.05% surfactant P20) (GE Healthcare; BR-1006-69). The flow cell was set at 25° C. The sample block was set at 16° C. Both were pretreated with the running buffer. In each cycle, first, the antibody to be tested was captured using the Protein A chip, and then a single concentration of CD33 antigen protein was injected. The association and dissociation processes of the antibody with the antigen protein were recorded, and finally, the chip was regenerated using Glycine pH 1.5 (GE Healthcare; BR-1003-54). The association was determined by injecting different concentrations of recombinant human CD33-his in the solution and maintaining for 240 s, wherein the flow rate was 30 μL/min, and the protein was diluted in a 1:1 dilution ratio from 200 nM to obtain 5 concentrations in total. The dissociation phase was monitored for up to 600 s and triggered by switching from the sample solution to the running buffer. The surface was regenerated by washing with 10 mM glycine solution (pH 1.5) at a flow rate of 30 μL/min for 30 s. The difference in bulk refractive index was corrected by subtracting the responses obtained from the goat anti-human Fc surface. Blank injections were also subtracted (=double reference). To calculate the apparent KD and other kinetic parameters, the Langmuir 1:1 model was used. The association rate (Kon), dissociation rate (Koff), and binding affinity (KD) of the VHH-Fc antibodies with the human CD33-his protein are shown in Tables 21 and 22, wherein the antibodies C331B904 and SGN-33 were used as positive controls. As shown in Tables 21 and 22, the affinity of VHH-Fc antibodies for human CD33 was superior to 1.40E-07 M.

TABLE 21
Assay on affinity of VHH-Fc antibodies
for human CD33 by SPR (biacore)

| Antibody name | Kon (1/Ms) | Koff (1/s) | KD (M) |
|---|---|---|---|
| S006-NB146-1 | 1.95E+05 | 4.38E−05 | 2.24E−10 |
| S006-NB146-3 | 2.49E+03 | 6.28E−06 | 2.52E−09 |
| S006-NB146-17 | 1.19E+05 | 2.18E−05 | 1.83E−10 |
| S006-NB146-20 | 4.12E+05 | 3.77E−04 | 9.17E−10 |
| S006-NB146-39 | 1.79E+05 | 2.95E−05 | 1.65E−10 |
| S006-NB146-40 | 1.46E+03 | 1.23E−05 | 8.39E−09 |
| S006-NB146-46 | 1.98E+05 | 3.39E−05 | 1.71E−10 |
| S006-NB146-60 | 1.49E+05 | 1.50E−04 | 1.01E−09 |
| S006-NB146-132 | 1.31E+05 | 6.96E−05 | 5.31E−10 |
| S006-NB147-225 | 2.93E+04 | 5.29E−04 | 1.80E−08 |
| C33B904 | 2.60E+05 | 4.08E−05 | 1.57E−10 |
| SGN-33 | 7.02E+05 | 5.57E−03 | 7.93E−09 |

TABLE 22
Assay on affinity of VHH-Fc antibodies
for human CD33 by SPR (biacore)

| Antibody name | Kon (1/Ms) | Koff (1/s) | KD (M) |
|---|---|---|---|
| S006-NB146-16 | 6.01E+04 | 2.24E−03 | 3.73E−08 |
| S006-NB147-25 | 1.41E+05 | 1.58E−04 | 1.12E−09 |
| S006-NB146-69 | 6.71E+04 | 3.26E−03 | 4.86E−08 |
| S006-NB146-139 | 2.97E+05 | 4.01E−03 | 1.35E−08 |
| S006-NB146-150 | 5.22E+04 | 7.33E−03 | 1.40E−07 |
| S006-NB146-173 | 7.25E+04 | 1.72E−03 | 2.38E−08 |
| S006-NB146-181 | 5.54E+04 | 2.87E−03 | 5.18E−08 |
| S006-NB147-220 | 1.81E+05 | 4.97E−04 | 2.75E−09 |
| C33B904 | 4.79E+05 | 7.44E−05 | 1.55E−10 |

### 7.2 Assay on Affinity of VHH-Fc Antibodies for Monkey CD33-his Protein

The assay on affinity of VHH-Fc antibodies for monkey CD33 protein (Sino Biological, Cat. No. 50712-M08H) was performed according to the method in Example 7.1. As shown in Tables 23 and 24, the affinity of S5006-NB146-20, S006-NB146-39, and S006-NB146-46 for monkey CD33 was superior to 1.0E-09 M.

TABLE 23
Assay on affinity of VHH-Fc antibodies
for monkey CD33 by SPR (biacore)

| Antibody name | Kon (1/Ms) | Koff (1/s) | KD (M) |
|---|---|---|---|
| S006-NB146-1 | 2.49E+05 | 2.68E−04 | 1.08E−09 |
| S006-NB146-3 | 1.08E+04 | 1.63E−04 | 1.51E−08 |
| S006-NB146-17 | 1.13E+05 | 1.28E−04 | 1.13E−09 |
| S006-NB146-20 | 9.02E+05 | 4.95E−04 | 5.48E−10 |
| S006-NB146-39 | 5.90E+05 | 6.98E−05 | 1.18E−10 |
| S006-NB146-40 | 1.39E+03 | 2.56E−04 | 1.83E−07 |
| S006-NB146-46 | 5.84E+05 | 9.36E−05 | 1.60E−10 |
| S006-NB146-132 | 2.16E+05 | 3.62E−04 | 1.67E−09 |
| C33B904 | 1.29E+06 | 6.32E−04 | 4.92E−10 |

TABLE 24
Assay on affinity of VHH-Fc antibodies
for monkey CD33 by SPR (biacore)

| Antibody name | Kon (1/Ms) | Koff (1/s) | KD (M) |
|---|---|---|---|
| S006-NB146-16 | 1.40E+05 | 1.54E−02 | 1.10E−07 |
| S006-NB147-25 | 4.06E+05 | 2.34E−03 | 5.76E−09 |
| S006-NB146-69 | 1.67E+05 | 1.19E−03 | 7.15E−09 |
| S006-NB146-139 | 9.25E+05 | 2.78E−02 | 3.01E−08 |
| S006-NB146-150 | 1.54E+05 | 6.40E−03 | 4.16E−08 |
| S006-NB146-173 | 1.55E+05 | 4.24E−03 | 2.74E−08 |
| S006-NB146-181 | 7.28E+04 | 1.98E−03 | 2.72E−08 |
| S006-NB147-220 | 6.08E+05 | 1.48E−03 | 2.43E−09 |
| C33B904 | 1.01E+06 | 4.97E−04 | 4.94E−10 |

### Example 8: Assay on Antibody-Antigen Binding Epitope

### 8.1 Identification of Antigen-Binding Regions of Antibodies

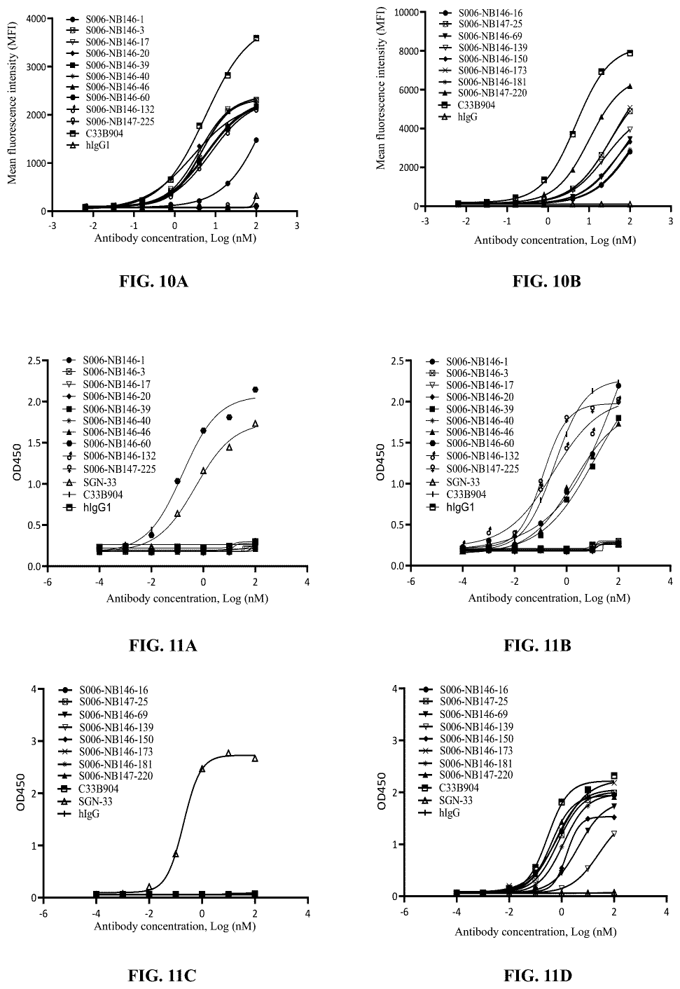

The extracellular region of CD33 protein has a conserved V-set immunoglobulin-like domain (V-domain) and a variable C2-set domain (C2-domain), wherein the V-domain is located at the membrane distal end and the C2-domain is located at the membrane proximal end. The antigen-binding epitope of SGN-33 is located at the V-domain, and the antigen-binding epitope of C331B904 is located at the C2-domain. In order to identify the distribution of antigen-binding epitopes of the VHH antibodies, plates were coated with human CD33-V-his (membrane distal end) and human CD33-C2-his (membrane proximal end), respectively, according to the ELISA method in Example 5.1. The VHH antibodies were classified based on the membrane distal end and the membrane proximal end, as shown in FIGS. 11A-11D and Table 25, which allowed the classification of the VHH antibodies into two classes:

TABLE 25
Classification of membrane distal
end and membrane proximal
end epitopes for VHH antibodies by ELISA

| | Binding region | |
|---|---|---|
| Antibody name | V-domain | C2-domain |
| S006-NB146-1 | − | + |
| S006-NB146-3 | − | − |
| S006-NB146-17 | − | − |
| S006-NB146-20 | − | − |
| S006-NB146-39 | − | + |
| S006-NB146-40 | − | − |
| S006-NB146-46 | − | + |
| S006-NB146-60 | + | − |
| S006-NB146-132 | − | + |
| S006-NB147-225 | − | + |
| S006-NB146-16 | − | + |
| S006-NB147-25 | − | + |
| S006-NB146-69 | − | + |
| S006-NB146-139 | − | + |
| S006-NB146-150 | − | + |
| S006-NB146-173 | − | + |
| S006-NB146-181 | − | + |
| S006-NB147-220 | − | + |

### 8.2 Antibody-Antigen Binding Epitope Binning

VHH antibodies and control antibodies with known epitopes were subjected to epitope classification with competitive ELISA. ELISA plates were coated with 2 μg/mL antibodies according to the method in Example 5.1; the human CD33-his protein was subjected to a gradient dilution from 30 μg/mL, and EC80 values (Tables 26-27) were calculated. ELISA plates were coated with 2 μg/mL antibodies; after 25 g/mL of antibodies to be tested were added, a human CD33-his protein having an EC80 concentration corresponding to each of the antibodies to be tested was then added for incubation for 2 h. The plates were washed 5 times with PBS, then HRP-labeled anti-His antibodies (purchased from GenScript, Cat. No. A00612) were added for incubation for 1 h, and the plates were washed 5 times. A TMB substrate was added at 50 μL/well and incubated at room temperature for 10 min, then a stop solution (1.0 M HCl) was added at 50 μL/well. OD_{450 nm} values were read using an ELISA plate reader (Insight, purchased from PerkinElmer), and the competition rate between the antibodies was calculated according to the OD_{450 nm} values using a formula. The results are shown in FIGS. 12A-12B, the higher the value of the competition rate, the closer the epitopes to which two antibodies bind.

TABLE 26
EC80 values of the human CD33-his
protein corresponding to VHH antibodies

| Antibody name | BC80 (MG/mL) |
|---|---|
| S006-NB146-1 | 0.08 |
| S006-NB146-3 | 0.12 |
| S006-NB146-17 | 0.09 |
| S006-NB146-20 | 0.07 |
| S006-NB146-39 | 0.06 |
| S006-NB146-40 | 0.10 |
| S006-NB146-46 | 0.07 |
| S006-NB146-60 | 0.05 |
| S006-NB146-132 | 0.06 |
| S006-NB147-225 | 0.09 |
| C33B904 | 0.03 |
| SGN-33 | 0.08 |

TABLE 27
EC80 values of the human CD33-his
protein corresponding to VHH antibodies

| Antibody name | EC80 (MG/mL) |
|---|---|
| S006-NB146-16 | 0.09 |
| S006-NB147-25 | 0.03 |
| S006-NB146-69 | 1.50 |
| S006-NB146-139 | 0.02 |
| S006-NB146-150 | 0.37 |
| S006-NB146-173 | 0.15 |
| S006-NB146-181 | 0.25 |
| S006-NB147-220 | 0.02 |
| C33B904 | 0.04 |
| SGN-33 | 0.03 |

### 8.3 Epitope Binning Between VHH-Fc Antibodies and Biotin-C331B904

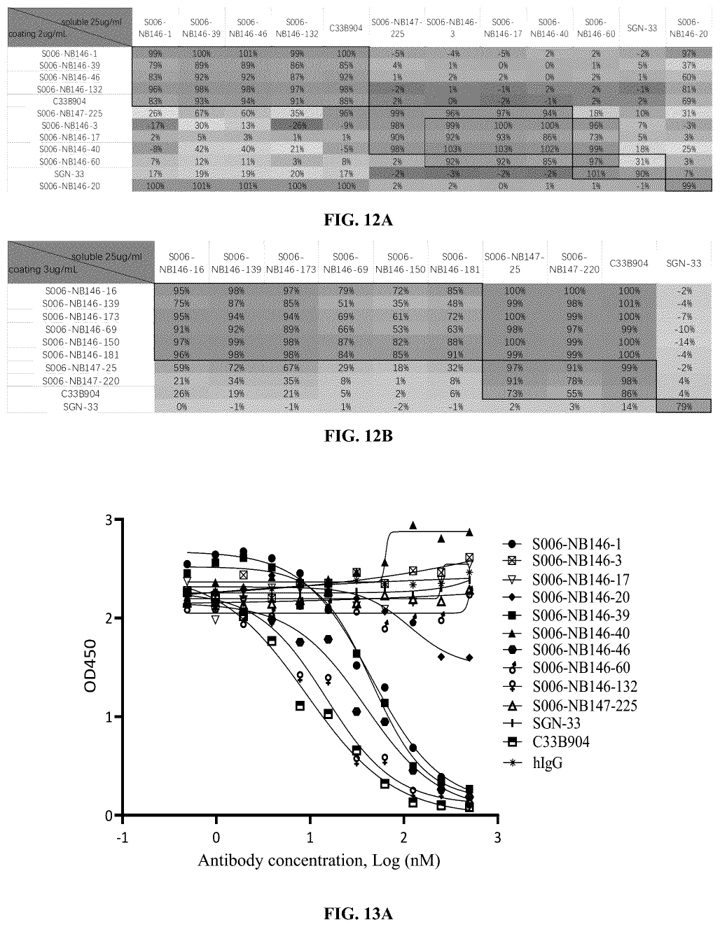

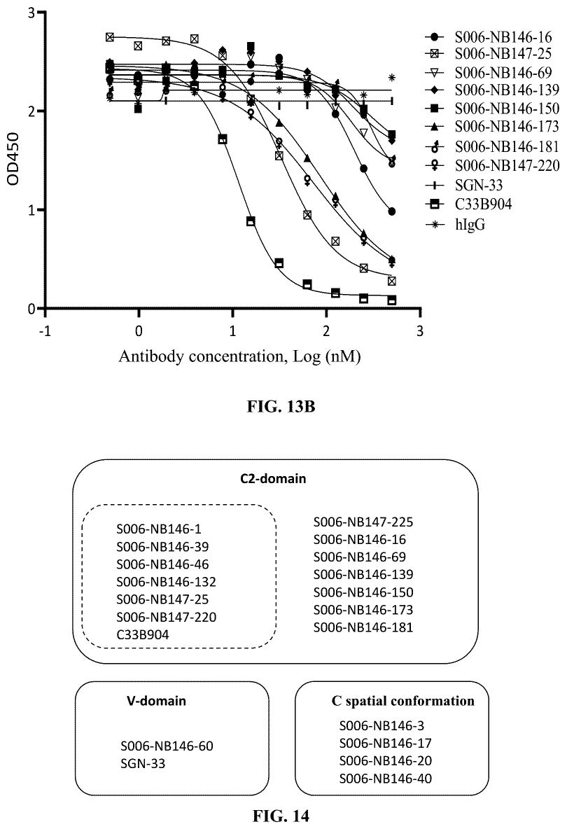

ELISA plates were coated with 2 μg/mL human CD33-his protein according to the method in Example 5.1; the Biotin-C331B904 antibody was subjected to a gradient dilution from 15 μg/mL, and EC80 values (0.18 μg/mL) were calculated. ELISA plates were coated with 2 μg/mL human CD33-his protein; starting from 40 μg/mL, the VHH antibodies to be tested were diluted in a 1:2 dilution ratio to obtain seven concentrations and added to the ELISA plates, and then Biotin-C331904 antibodies with the EC80 concentrations were added for incubation for 2 h. The plates were washed 5 times with PBS and then HRP-labeled anti-Streptavidin antibodies (purchased from Sigma, Cat. No. S2438) were added for incubation for 1 h, and the plates were washed 5 times. A TMB substrate was added at 50 μL/well and incubated at room temperature for 10 min, then a stop solution (1.0 M HCl) was added at 50 μL/well. OD_{450 nm} values were read using an ELISA plate reader (Insight, purchased from PerkinElmer). Then, analysis was performed by software (GraphPad Prism8), and data were fitted for curve plotting. The results are shown in FIGS. 13A-13B and Tables 28 and 29.

TABLE 28
Results of epitope binning between VHH-hFc antibodies and Biotin-C33B904

| Competitive | Concentration (nM) | | | | | | | | | | | |
|---|---|---|---|---|---|---|---|---|---|---|---|---|
| Antibodies | 500 | 250.00 | 125.00 | 62.50 | 31.25 | 15.63 | 7.81 | 3.91 | 1.95 | 0.98 | 0.49 | 0 |
| S006-NB146-1 | 0.26 | 0.39 | 0.68 | 1.30 | 1.52 | 2.08 | 2.45 | 2.61 | 2.68 | 2.64 | 2.55 | 2.67 |
| S006-NB146-3 | 2.61 | 2.46 | 2.48 | 2.35 | 2.46 | 2.35 | 2.35 | 2.20 | 2.44 | 2.12 | 2.28 | 2.35 |
| S006-NB146-17 | 2.55 | 2.38 | 2.16 | 2.09 | 2.12 | 2.25 | 2.30 | 2.30 | 2.18 | 1.98 | 2.37 | 2.45 |
| S006-NB146-20 | 1.60 | 1.61 | 1.96 | 2.07 | 2.20 | 2.33 | 2.33 | 2.43 | 2.29 | 2.27 | 2.25 | 2.33 |
| S006-NB146-39 | 0.26 | 0.34 | 0.50 | 1.14 | 1.64 | 2.25 | 2.13 | 2.51 | 2.61 | 2.56 | 2.45 | 2.51 |
| S006-NB146-40 | 2.87 | 2.81 | 2.94 | 2.57 | 2.47 | 2.39 | 2.40 | 2.42 | 2.31 | 2.36 | 2.21 | 2.29 |
| S006-NB146-46 | 0.19 | 0.26 | 0.45 | 0.95 | 1.05 | 1.78 | 1.76 | 1.99 | 2.07 | 2.16 | 2.14 | 2.24 |
| S006-NB146-60 | 2.26 | 2.00 | 1.98 | 1.92 | 2.09 | 2.13 | 2.15 | 2.05 | 1.96 | 2.11 | 2.11 | 2.18 |
| S006-NB146-132 | 0.11 | 0.22 | 0.23 | 0.56 | 0.55 | 1.37 | 1.40 | 1.97 | 2.17 | 2.12 | 2.23 | 2.27 |
| S006-NB147-225 | 2.29 | 2.17 | 2.19 | 2.23 | 2.15 | 2.27 | 2.18 | 2.14 | 2.13 | 2.16 | 2.16 | 2.27 |
| SGN-33 | 2.39 | 2.31 | 2.24 | 2.30 | 2.27 | 2.35 | 2.25 | 2.22 | 2.23 | 2.23 | 2.23 | 2.30 |
| C33B904 | 0.08 | 0.10 | 0.13 | 0.32 | 0.66 | 1.03 | 1.11 | 1.77 | 2.02 | 2.22 | 2.26 | 2.33 |
| hIgG1 | 2.46 | 2.35 | 2.34 | 2.35 | 2.38 | 2.35 | 2.33 | 2.30 | 2.30 | 2.27 | 2.22 | 2.24 |

TABLE 29
Results of epitope binning between VHH-hFc antibodies and Biotin-C33B904

| Competitive | Concentration (nM) | | | | | | | | | | | |
|---|---|---|---|---|---|---|---|---|---|---|---|---|
| Antibodies | 500 | 250.00 | 125.00 | 62.50 | 31.25 | 15.63 | 7.81 | 3.91 | 1.95 | 0.98 | 0.49 | 0 |
| S006-NB146-16 | 0.98 | 1.42 | 1.97 | 2.21 | 2.54 | 2.47 | 2.42 | 2.40 | 2.41 | 2.36 | 2.32 | 2.36 |
| S006-NB147-25 | 0.28 | 0.41 | 0.68 | 0.95 | 1.55 | 2.12 | 2.56 | 2.73 | 2.71 | 2.66 | 2.75 | 2.34 |
| S006-NB146-69 | 1.49 | 1.78 | 2.03 | 2.32 | 2.43 | 2.55 | 2.33 | 2.34 | 2.36 | 2.14 | 2.39 | 2.51 |
| S006-NB146-139 | 1.70 | 1.98 | 2.16 | 2.40 | 2.50 | 2.60 | 2.62 | 2.49 | 2.40 | 2.22 | 2.49 | 2.60 |
| S006-NB146-150 | 1.76 | 2.02 | 2.22 | 2.24 | 2.36 | 2.66 | 2.41 | 2.38 | 2.31 | 2.02 | 2.48 | 2.54 |
| S006-NB146-173 | 0.50 | 0.76 | 1.14 | 1.52 | 1.88 | 2.10 | 2.38 | 2.47 | 2.47 | 2.36 | 2.42 | 2.50 |
| S006-NB146-181 | 1.49 | 1.99 | 2.26 | 2.30 | 2.41 | 2.33 | 2.27 | 2.40 | 2.23 | 2.22 | 2.19 | 2.24 |
| S006-NB147-220 | 0.46 | 0.69 | 1.07 | 1.29 | 1.67 | 1.96 | 2.14 | 2.33 | 2.27 | 2.29 | 2.30 | 2.37 |
| SGN-33 | 2.10 | 2.07 | 2.09 | 2.06 | 2.05 | 2.08 | 2.13 | 2.20 | 2.10 | 2.02 | 2.19 | 2.27 |
| C33B904 | 0.08 | 0.10 | 0.16 | 0.24 | 0.46 | 0.88 | 1.72 | 2.25 | 2.43 | 2.31 | 2.44 | 2.35 |
| hIgG1 | 2.34 | 2.16 | 2.17 | 2.17 | 2.21 | 2.30 | 2.17 | 2.19 | 2.21 | 2.08 | 2.13 | 2.26 |

According to the results of the above two competitive methods and the binding experiment with two truncated proteins, VHH antibodies were classified, and the results are shown in FIG. 14. S006-N B146-1, S006-NB146-3 9, S006-NB146-46, S006-NB146-13 2, S006-NB147-25, and S006-NB147-220 competed with C331B904 (binding epitope in C2 domain), and thus they were classified into one class; S006-NB147-225 could bind to the human CD33-C2-his protein but did not compete with C331B904; S006-NB146-16, S006-NB146-69, S006-NB146-139, S006-NB146-150, S006-NB146-173, and S006-NB146-181 could bind to the human CD33-C2-his protein and competed with C33B904 incompletely, and thus, they were determined to belong to C2 domain but were not exactly the same as C33B904 in terms of epitope; S006-NB146-60 and SGN-33 could both bind to the human CD33-V-his protein, and thus they were classified into one class; S006-NB146-3, S006-NB1 46-17, S006-NB146-20, and S006-NB146-40 could neither bind to the human CD33-C2-his protein nor bind to the human CD33-V-his protein, and thus they were classified into one class (spatial conformation).

### Example 9: Humanization of CD33 VHH Single Domain Antibodies

### 9.1 Humanization Design of CD33 VHH Single Domain Antibodies

By comparing the IMGT database (website: imgt.cines.fr) for germline genes from heavy and light chain variable regions of human antibodies, heavy chain variable region germline genes IGHV3-7*01 and IGHJ3*01, with high homology with the CD33 VHH single domain antibodies, were selected as templates, and CDRs of the CD33 VHH single domain antibodies S006-NB146-17, S006-NB146-39, S006-NB146-60, S006-NB146-132, S006-NB146-173, and S006-NB147-225 were respectively grafted into corresponding humanized templates to form variable region sequences in the following order: FR1-CDR1-FR2-CDR2-FR3-CDR3-FR4. Key amino acids in the framework sequences were back-mutated into amino acids corresponding to the single domain antibodies to ensure the original affinity. Point mutations were performed on sites (e.g., DD, NSS) that were susceptible to chemical modification, existing in the antibodies, so as to eliminate modification risks. Compared to the heavy chain template IGHV3-7*01, two amino acids 77N and 78A were deleted in the FR3 sequence of the llama antibody S006-NB146-39, and the two newly introduced amino acids (NA) were removed in the humanized antibody S006-NB146-39. See Tables 30-35 for specific mutation design.

TABLE 30
Mutation design for S006-NB146-17 humanized antibodies

| | | CDR | CDR region |
|---|---|---|---|
| | | region | definition |
| VHH name | Framework region mutations | mutations | scheme |
| S006-NB146- | S39G, V41Y, G48Q, | None | IMGT |
| 17-H1 | L49R, N54T | | |
| S006-NB146- | S39G, V41Y, G48Q, | None | IMGT |
| 17-H2 | L49R, N54T, Y62N | | |
| S006-NB146- | S39G, V41Y, G48Q, L49R, | None | IMGT |
| 17-H3 | N54T, Y62N, L82A | | |
| S006-NB146- | S39G, V41Y, G48Q, L49R, | None | IMGT |
| 17-H4 | N54T, Y62N, L82A + E1Q | | |
| S006-NB146- | S39G, V41Y, G48Q, L49R, | D32Q | IMGT |
| 17-H5 | N54T, Y62N, L82A | | |
| S006-NB146- | S39G, V41Y, G48Q, L49R, | None | IMGT |
| 17-H6 | N54T, Y62N, V64G, L82A | | |
| S006-NB146- | S39G, V41Y, G48Q, L49R, | None | IMGT |
| 17-H7 | N54T, Y62N, N77S, L82A | | |
| S006-NB146- | S39G, V41Y, G48Q, L49R, | D32N | IMGT |
| 17-H8 | N54T, Y62N, N77S, L82A | | |
| S006-NB146- | S39G, V41Y, G48Q, L49R, | None | IMGT |
| 17-H9 | N54T, Y62N, L82A, R90K, | | |
| | A91P | | |

Note:
S39G indicates the mutation of S to G at position 39, others and so on, with the amino acids numbered in the natural order.

TABLE 31
Mutation design for S006-NB146-39 humanized antibodies

| | | | CDR |
|---|---|---|---|
| | | CDR | region |
| | | region | definition |
| VHH name | Framework region mutations | mutations | scheme |
| S006-NB146- | F27I/T28I/F29L/S30D/ | S115A | Kabat |
| 39-aH8 | V37F/G44E/L45R/W47G/ | | |
| | R99A/del77N/del78A | | |
| S006-NB146- | F27I/T28I/F29L/S30D/ | D56S/ | Kabat |
| 39-aH9 | V37F/G44E/L45R/W47G/ | S115A | |
| | R99A/del77N/del78A | | |
| S006-NB146- | A23T/S25P/F27I/T28I/F29L/ | S115A | Kabat |
| 39-aH10 | S30D/V37F/G44E/L45R/W47G/ | | |
| | R99A/del77N/del78A | | |
| S006-NB146- | A23T/S25P/F27I/T28I/F29L/ | D56S/ | Kabat |
| 39-aH11 | S30D/V37F/G44E/L45R/W47G/ | S115A | |
| | R99A/del77N/del78A | | |
| S006-NB146- | F271/T281/F29L/S30D/V37F/ | S115A | Kabat |
| 39-aH12 | G44E/L45R/W47G/D76E/S79T/ | | |
| | R99A/del77N/del78A | | |
| S006-NB146- | F27I/T281/F29L/S30D/V37F/ | D56S/ | Kabat |
| 39-aH13 | G44E/L45R/W47G/D76E/S79T/ | S115A | |
| | R99A/del77N/del78A/ | | |
| S006-NB146- | F271/T281/F29L/S30D/ | S115A | Kabat |
| 39-aH15 | V37F/L45R/W47G/R99A/ | | |
| | del77N/de178A | | |

Note:
F27I indicates the mutation of F to I at position 27, others and so on, del77N indicates the deletion of N at position 77, and del78A indicates the deletion of A at position 78, with the amino acids numbered in the natural order.

TABLE 32
Mutation design for S006-NB146-60 humanized antibodies

| | | CDR | CDR region |
|---|---|---|---|
| | | region | definition |
| VHH name | Framework region mutations | mutations | scheme |
| S006-NB146- | V37H/G44Q/L45R/ | None | IMGT |
| 60-H3 | W47L/N50S/Y58 | | |
| S006-NB146- | V37H/G44Q/L45R/W47L/ | None | IMGT |
| 60-H4 | N50S/Y58N/169M/L78V | | |
| S006-NB146- | V37H/G44Q/L4SR/W47L/ | None | IMGT |
| 60-H5 | NS0S/Y58N/N73D | | |
| S006-NB146- | V37H/G44Q/L45R/W47L/ | None | IMGT |
| 60-H6 | N50S/Y58N/N73D /D72N | | |
| S006-NB146- | V42H/G49Q/L50R/W52L/ | None | IMGT |
| 60-H7 | N55S/Y66N/R95K/A96P | | |

Note:
V37H indicates the mutation of V to H at position 37, others and so on, with the amino acids numbered in the natural order.

TABLE 33
Mutation design for S006-NB146-132 humanized antibodies

| | | CDR | CDR region |
|---|---|---|---|
| | | region | definition |
| VHH name | Framework region mutations | mutations | scheme |
| S006-NB146- | V36Y/G43Q/L44R/ | N49S | Kabat |
| 132-aH1 | W46L/A95R/R96L | | |
| S006-NB146- | F27G/S30V/V36Y/G43Q/ | N49S | Kabat |
| 132-aH2 | L44R/W46L/A95R/R96L | | |
| S006-NB146- | F27G/S30V/V36Y/G43Q/ | N49S | Kabat |
| 132-aH3 | L44R/W46L/A95R/R96L/EIQ | | |
| S006-NB146- | F27G/S30V/V36Y/G43Q/ | N49S | Kabat |
| 132-aH5 | L44R/W46L/L77V/A95R/R96L | | |

Note:
V36Y indicates the mutation of V to Y at position 36, others and so on, with the amino acids numbered in the natural order.

TABLE 34
Mutation design for S006-NB146-173 humanized antibodies

| | | CDR | CDR |
|---|---|---|---|
| | | | region |
| | | region | definition |
| VHH name | Framework region mutations | mutations | scheme |
| S006-NB146- | V37Y/G44Q/L45R/ | None | Kabat |
| 173-H2 | W47L/A96N/R97A | | |
| S006-NB146- | F27S/V37Y/G44Q/L45R/ | | Kabat |
| 173-H3 | W47L/A96N/R97A | | |
| S006-NB146- | F27S/F29L/V37Y/G44Q/ | None | Kabat |
| 173-H4 | L45R/W47L/A96N/R97A | | |
| S006-NB146- | F27S/F29L/V37Y/G44Q/L45R/ | None | Kabat |
| 173-H5 | W47L/N73G/A74T/A96N, R97A | | |
| S006-NB146- | F27S/F29L/V37Y/G44Q/L45R/ | None | Kabat |
| 173-H6 | W47L/N73G/A74T/L78V/ | | |
| | A96N/R97A | | |

Note:
V37Y indicates the mutation of V to Y at position 37, others and so on, with the amino acids numbered in the natural order.

TABLE 35
Back mutation design for S006-NB147-225 humanized antibodies

| | | CDR | CDR region |
|---|---|---|---|
| | | region | definition |
| VHH name | Back mutations | mutations | scheme |
| S006-NB147- | L4V/R95L/A96P/V101T/ | None | Kabat |
| 225-H5 | W118Q/M123Q | | |

Note:
LAV indicates the mutation of L to V at position 4, others and so on, with the amino acids numbered in the natural order.

### 9.2 Specific Sequences of the Variable Regions of S006-NB146-17 Humanized Antibodies

S006-NB146-17-H1:
(SEQ ID NO: 193)
EVQLVESGGGLVQPGGSLRLSCAASGSIFTSDDMDYAMGWYRQAPGKQRE
WVATITSDEDTYYVDSVKGRFTISRDNAKNSLYLQMNSLRAEDTAVYYCF
ANVRSDRSSLYRNYWGQGTMVTVSS.
S006-NB146-17-H2:
(SEQ ID NO: 194)
EVQLVESGGGLVQPGGSLRLSCAASGSIFTSDDMDYAMGWYRQAPGKQRE
WVATITSDEDTNYVDSVKGRFTISRDNAKNSLYLQMNSLRAEDTAVYYCF
ANVRSDRSSLYRNYWGQGTMVTVSS.
S006-NB146-17-H3:
(SEQ ID NO: 195)
EVQLVESGGGLVQPGGSLRLSCAASGSIFTSDDMDYAMGWYRQAPGKQRE
WVATITSDEDTNYVDSVKGRFTISRDNAKNSAYLQMNSLRAEDTAVYYCF
ANVRSDRSSLYRNYWGQGTMVTVSS.
S006-NB146-17-H4:
(SEQ ID NO: 196)
QVQLVESGGGLVQPGGSLRLSCAASGSIFTSDDMDYAMGWYRQAPGKQRE
WVATITSDEDTNYVDSVKGRFTISRDNAKNSAYLQMNSLRAEDTAVYYCF
ANVRSDRSSLYRNYWGQGTMVTVSS.
S006-NB146-17-H5:
(SEQ ID NO: 197)
EVQLVESGGGLVQPGGSLRLSCAASGSIFTSQDMDYAMGWYRQAPGKQRE
WVATITSDEDTNYVDSVKGRFTISRDNAKNSAYLQMNSLRAEDTAVYYCF
ANVRSDRSSLYRNYWGQGTMVTVSS.
S006-NB146-17-H6:
(SEQ ID NO: 198)
EVQLVESGGGLVQPGGSLRLSCAASGSIFTSDDMDYAMGWYRQAPGKQRE
WVATITSDEDTNYGDSVKGRFTISRDNAKNSAYLQMNSLRAEDTAVYYCF
ANVRSDRSSLYRNYWGQGTMVTVSS.
S006-NB146-17-H7:
(SEQ ID NO: 199)
EVQLVESGGGLVQPGGSLRLSCAASGSIFTSDDMDYAMGWYRQAPGKQRE
WVATITSDEDTNYVDSVKGRFTISRDSAKNSAYLQMNSLRAEDTAVYYCF
ANVRSDRSSLYRNYWGQGTMVTVSS.
S006-NB146-17-H8:
(SEQ ID NO: 200)
EVQLVESGGGLVQPGGSLRLSCAASGSIFTSNDMDYAMGWYRQAPGKQRE
WVATITSDEDTNYVDSVKGRFTISRDSAKNSAYLQMNSLRAEDTAVYYCF
ANVRSDRSSLYRNYWGQGTMVTVSS.
S006-NB146-17-H9:
(SEQ ID NO: 201)
EVQLVESGGGLVQPGGSLRLSCAASGSIFTSDDMDYAMGWYRQAPGKQRE
WVATITSDEDTNYVDSVKGRFTISRDNAKNSAYLQMNSLKPEDTAVYYCF
ANVRSDRSSLYRNYWGQGTMVTVSS.

### 9.3 Specific Sequences of the Variable Regions of S006-NB146-39 Humanized Antibodies

S006-NB146-39-aH8:
(SEQ ID NO: 202)
EVQLVESGGGLVQPGGSLRLSCAASGIILDYYPIGWFRQAPGKEREGVAF
ITNSDDSTIYTNYADSVKGRFTISRDKNSLYLQMNSLRAEDTAVYYCAAR
STYIVGRNENALNSADYDYWGQGTMVTVSS.
S006-NB146-39-aH9:
(SEQ ID NO: 203)
EVQVESGGGLVQPGGSLRLSCAASGIILDYYPIGWFRQAPGKEREGVAF
ITNSDSSTIYTNYADSVKGRFTISRDKNSLYLQMNSLRAEDTAVYYCAAR
STYIVGRNENALNSADYDYWGQGTMVTVSS.
S006-NB146-39-aH10:
(SEQ ID NO: 204)
EVQLVESGGGLVQPGGSLRLSCTAPGIILDYYPIGWFRQAPGKEREGVAF
ITNSDDSTIYTNYADSVKGRFTISRDKNSLYLQMNSLRAEDTAVYYCAAR
STYIVGRNENALNSADYDYWGQGTMVTVSS.
S006-NB146-39-aH11;
(SEQ ID NO: 205)
EVQLVESGGGLVQPGGSLRLSCTAPGIILDYYPIGWFRQAPGKEREGVAF
ITNSDSSTIYTNYADSVKGRFTISRDKNSLYLQMNSLRAEDTAVYYCAAR
STYIVGRNENALNSADYDYWGQGTMVTVSS.
S006-NB146-39-aH12:
(SEQ ID NO: 206)
EVQLVESGGGLVQPGGSLRLSCAASGIILDYYPIGWFRQAPGKEREGVAF
ITNSDDSTIYTNYADSVKGRFTISREKNTLYLQMNSLRAEDTAVYYCAAR
STYIVGRNENALNSADYDYWGQGTMVTVSS.
S006-NB146-39-aH13:
(SEQ ID NO: 207)
EVOLVESGGGLVQPGGSLRLSCAASGIILDYYPIGWFRQAPGKEREGVAF
ITNSDSSTIYTNYADSVKGRFTISREKNTLYLQMNSLRAEDTAVYYCAAR
STYIVGRNENALNSADYDYWGQGTMVTVSS.
S006-NB146-39-aH15:
(SEQ ID NO: 208)
EVQLVESGGGLVQPGGSLRLSCAASGIILDYYPIGWFRQAPGKGREGVAF
ITNSDDSTIYTNYADSVKGRFTISRDKNSLYLQMNSLRAEDTAVYYCAAR
STYIVGRNENALNSADYDYWGQGTMVTVSS.

### 9.4 Specific Sequences of the Variable Regions of S006-NB146-60 Humanized Antibodies

S006-NB146-60-H3:
(SEQ ID NO: 209)
EVQLVESGGGLVQPGGSLRLSCAASRSTSSLSLMSWHRQAPGKQRELVAS
ITSGGKANYVDSVKGRFTISRDNAKNSLYLQMNSLRAEDTAVYYCHAYDR
AGTASGFGAAWGQGTMVTVSS.
S006-NB146-60-H4:
(SEQ ID NO: 210)
EVQLVESGGGLVQPGGSLRLSCAASRSTSSLSLMSWHRQAPGKQRELVAS
ITSGGKANYVDSVKGRFTMSRDNAKNSVYLQMNSLRAEDTAVYYCHAYDR
AGTASGFGAAWGQGTMVTVSS.
S006-NB146-60-H5:
(SEQ ID NO: 211)
EVQLVESGGGLVQPGGSLRLSCAASRSTSSLSLMSWHRQAPGKQRELVAS
ITSGGKANYVDSVKGRFTISRDDAKNSLYLQMNSLRAEDTAVYYCHAYDR
AGTASGFGAAWGQGTMVTVSS.
S006-NB146-60-H6:
(SEQ ID NO: 212)
EVQLVESGGGLVQPGGSLRLSCAASRSTSSLSLMSWHRQAPGKQRELVAS
ITSGGKANYVDSVKGRFTISRNDAKNSLYLQMNSLRAEDTAVYYCHAYDR
AGTASGFGAAWGQGTMVTVSS.
S006-NB146-60-H7:
(SEQ ID NO: 213)
EVQLVESGGGLVQPGGSLRLSCAASRSTSSLSLMSWHRQAPGKQRELVAS
ITSGGKANYVDSVKGRFTISRDNAKNSLYLQMNSLKPEDTAVYYCHAYDR
AGTASGFGAAWGQGTMVTVSS.

### 9.5 Specific Sequences of the Variable Regions of S006-NB146-132 Humanized Antibodies

S006-NB146-132-aH1:
(SEQ ID NO: 214)
EVQLVESGGGLVQPGGSLRLSCAASGFTFSINAAWYRQAPGKQRELVASI
SYGGSTNYADRVAGRFTISRDNAKNSLYLQMNSLRAEDTAVYYCRLDPGL
ALNNRPLYYWGQGTMVTVSS.
S006-NB146-132-aH2:
(SEQ ID NO: 215)
EVQLVESGGGLVQPGGSLRLSCAASGGTFVINAAWYRQAPGKQRELVASI
SYGGSTNYADRVAGRFTISRDNAKNSLYLQMNSLRAEDTAVYYCRLDPGL
ALNNRPLYYWGQGTMVTVSS.
S006-NB146-132-aH3:
(SEQ ID NO: 216)
QVQLVESGGGLVQPGGSLRLSCAASGGTFVINAAWYRQAPGKQRELVASI
SYGGSTNYADRVAGRFTISRDNAKNSLYLQMNSLRAEDTAVYYCRLDPGL
ALNNRPLYYWGQGTMVTVSS.
S006-NB146-132-aH5:
(SEQ ID NO: 217)
EVQLVESGGGLVQPGGSLRLSCAASGGTFVINAAWYRQAPGKQRELVASI
SYGGSTNYADRVAGRFTISRDNAKNSVYLQMNSLRAEDTAVYYCRLDPGL
ALNNRPLYYWGQGTMVTVSS

### 9.6 Specific Sequences of the Variable Regions of S006-NB146-173 Humanized Antibodies

S006-NB146-173-H2:
(SEQ ID NO: 218)
EVQLVESGGGLVQPGGSLRLSCAASGFTFSLHSMGWYRQAPGKQRELVAG
ISRDGDSNYLSSLAGRFTISRDNAKNSLYLQMNSLRAEDTAVYYCNARTS
PWSAALWGQGTMVTVSS.
S006-NB146-173-H3:
(SEQ ID NO: 219)
EVQLVESGGGLVQPGGSLRLSCAASGSTFSLHSMGWYRQAPGKQRELVAG
ISRDGDSNYLSSLAGRFTISRDNAKNSLYLQMNSLRAEDTAVYYCNARTS
PWSAALWGQGTMVTVSS.
S006-NB146-173-H4;
(SEQ ID NO: 220)
EVQLVESGGGLVQPGGSLRLSCAASGSTLSLHSMGWYRQAPGKQRELVAG
ISRDGDSNYLSSLAGRFTISRDNAKNSLYLQMNSLRAEDTAVYYCNARTS
PWSAALWGQGTMVTVSS.
S006-NB146-173-H5:
(SEQ ID NO: 221)
EVQLVESGGGLVQPGGSLRLSCAASGSTLSLHSMGWYRQAPGKQRELVAG
ISRDGDSNYLSSLAGRFTISRDGTKNSLYLQMNSLRAEDTAVYYCNARTS
PWSAALWGQGTMVTVSS.
S006-NB146-173-H6:
(SEQ ID NO: 222)
EVQLVESGGGLVQPGGSLRLSCAASGSTLSLHSMGWYRQAPGKQRELVA
GISRDGDSNYLSSLAGRFTISRDGTKNSVYLQMNSLRAEDTAVYYCNAR
TSPWSAALWGQGTMVTVSS.

### 9.7 Specific Sequences of the Variable Regions of S006-NB146-225 Humanized Antibodies

S006-NB147-225-HS:
(SEQ ID NO: 223)
EVQVVESGGGLVQPGGSLRLSCAASGFTFSNYAMSWVRQAPGKGLEWVAE
INSGGDNTYYAYFVKGRFTISRDNAKNSLYLQMNSLKPEDTATYYCAREE
ENAGTILQGQGTQVTVSS

### 9.8 Humanized Template Sequences for the CD33 VHH Single Domain Antibodies

Humanized heavy chain template IGHV3-7*01:
(SEQ ID NO: 224)
EVQLVESGGGLVQPGGSLRLSCAASGFTFSSYWMSWVRQAPGKGLEWVAN
IKQDGSEKYYVDSVKGRFTISRDNAKNSLYLQMNSLRAEDTAVYYCAR
Humanized heavy chain template IGHJ3*01:
(SEQ ID NO: 225)
WGQGTMVTVSS.

### 9.9 CDR Region Sequences of CD33 Humanized Antibodies (Table 36-Table 41)

TABLE 36
IMGT analysis results for VHH sequences of S006-NB146-17
humanized antibodies

| Variable region | CDRI | CDR2 | CDR3 |
|---|---|---|---|
| S006-NB146-17-H1 | GSIFTSDDMDYA | ITSDEDT | FANVRSDRSSLYRNY |
| S006-NB146-17-H2 | (SEQ ID NO: 143) | (SEQ ID NO: 144) | (SEQ ID NO: 145) |
| S006-NB146-17-H3 | | | |
| S006-NB146-17-H4 | | | |
| S006-NB146-17-H6 | | | |
| S006-NB146-17-H7 | | | |
| S006-NB146-17-H9 | | | |
| S006-NB146-17-H5 | GSIFTSQDMDYA | ITSDEDT | FANVRSDRSSLYRNY |
| | (SEQ ID NO: 226) | (SEQ ID NO: 144) | (SEQ ID NO: 145) |
| S006-NB146-17-H8 | GSIFTSNDMDYA | ITSDEDT | FANVRSDRSSLYRNY |
| | (SEQ ID NO: 227) | (SEQ ID NO: 144) | (SEQ ID NO: 145) |

TABLE 37
Kabat analysis results for VH sequences of S006-NB146-39
humanized antibodies

| Variable region | CDRI | CDR2 | CDR3 |
|---|---|---|---|
| S006-NB146-39-aH8 | YYPIG | FITNSDDSTIYTNYADSV | RSTYIVGRNENALNSADYD |
| S006-NB146-39-aH10 | (SEQ ID NO: 41) | KG | Y |
| S006-NB146-39-aH12 | | (SEQ ID NO: 42) | (SEQ ID NO: 228) |
| S006-NB146-39-aH15 | | | |
| S006-NB146-39-aH9 | YYPIG | FITNSDSSTIYTNYADSVK | RSTYIVGRNENALNSADYD |
| S006-NB146-39-aH11 | (SEQ ID NO: 41) | G | Y |
| S006-NB146-39-aH13 | | (SEQ ID NO: 229) | (SEQ ID NO: 228) |

TABLE 38
IMGT analysis results for VH sequences of S006-NB146-60
humanized antibodies

| Variable region | CDR1 | CDR2 | CDR3 |
|---|---|---|---|
| S006-NB146-60-H3 | RSTSSLSL | ITSGGKA | HAYDRAGTASGVFGAA |
| S006-NB146-60-H4 | (SEQ ID NO: 158) | (SEQ ID NO: 159) | (SEQ ID NO: 160) |
| S006-NB146-60-H5 | | | |
| S006-NB146-60-H6 | | | |
| S006-NB146-60-H7 | | | |

TABLE 39
Kabat analysis results for VH sequences of S006-NB146-132
humanized antibodies

| Variable region | CDR1 | CDR2 | CDR3 |
|---|---|---|---|
| NB146-132-aHl | INAA | SISYGGSTNYADRVAG | DPGLALNNRPLYY |
| NB146-132-aH2 | (SEQ ID NO: 53) | (SEQ ID NO: 230) | (SEQ ID NO: 55) |
| NB146-132-aH3 | | | |
| NB146-132-aH5 | | | |

TABLE 40
Kabat analysis results for VH sequences of S006-NB146-173
humanized antibodies

| Variable region | CDR1 | CDR2 | CDR3 |
|---|---|---|---|
| S006-NB146-173-H2 | LHSMG | GISRDGDSNYLSSLAG | RTSPWSAAL |
| S006-NB146-173-H3 | (SEQ ID NO: 74) | (SEQ ID NO: 75) | (SEQ ID NO: 76) |
| S006-NB146-173-H4 | | | |
| S006-NB146-173-H5 | | | |
| S006-NB146-173-H6 | | | |

TABLE 41
Kabat analysis results for VH sequences of S006-NB147-225
humanized antibodies

| Variable region | CDR1 | CDR2 | CDR3 |
|---|---|---|---|
| S006-NB147-225-H5 | NYAMS | EINSGGDNTYYAYFVKG | EEENAGTIL |
| | (SEQ ID NO: 56) | (SEQ ID NO: 57) | (SEQ ID NO: 58) |

### 9.10 Preparation of CD33 VHH Humanized Antibodies

The variable region sequences of the aforementioned VHH humanized antibodies in this example were each recombined into an expression vector B13.4-huIgG1 containing a signal peptide and human IgG1 Fc, and humanized VHH-Fc antibodies were prepared according to the method described in Example 4.1.

### Example 10: Identification of VHH-Fc Humanized Antibodies

### 10.1 Assay on Binding of VHH-Fc Humanized Antibodies to CD33 Protein by Enzyme-Linked Immunosorbent Assay (ELISA)

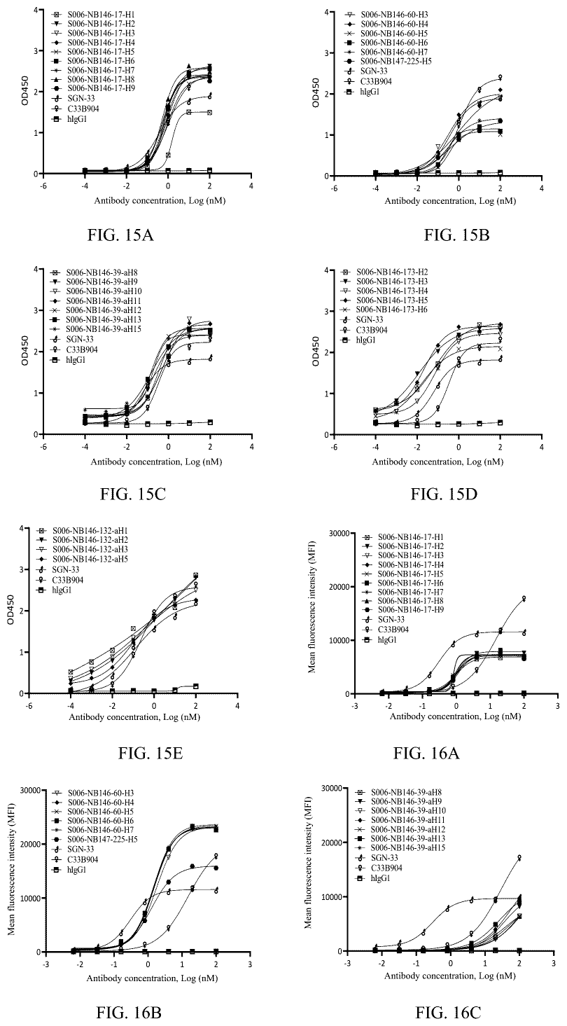

The binding activity of the VHH-Fc humanized antibodies to the human CD33 protein was assayed with reference to the method described in Example 5.1. The assay results for the binding of VHH humanized antibodies to hCD33-his by ELISA are shown in FIGS. 15A-15E. The results show that all the VHH humanized antibodies can bind to the human CD33 protein at the ELISA level.

### 10.2 Assay on Binding of VHH-Fc Humanized Antibodies to Different CD33-Expressing Cells by Flow Cytometry Assay (FACS)

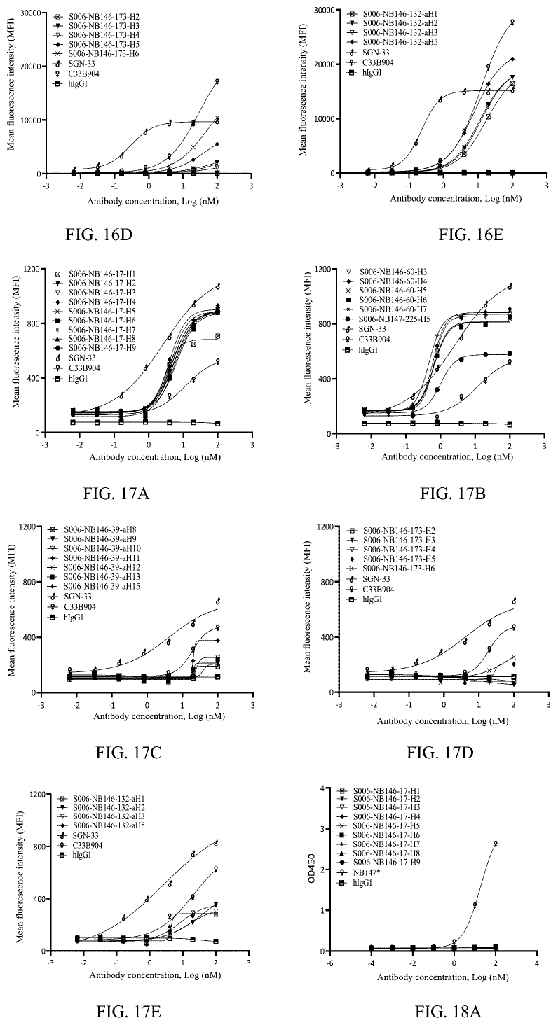

The binding of the VHH-Fc humanized antibodies to different CD33-expressing cells was assayed with reference to the method described in Example 5.2. The analysis results are shown in FIGS. 16A-16E and FIGS. 17A-17E, indicating that all the VHH humanized antibodies can bind to the human CD33 protein on the surface of CHO-K1-human CD33-B8 cells and U937 cells, and all the VHH humanized antibodies do not bind to negative cells Jurkat and CHO-K1 null cells.

### Example 11: Assay on Species Cross-Binding Activity of VHH-Fc Humanized Antibodies

### 11.1 Assay on Binding of VHH-Fc Humanized Antibodies to CD33 Proteins of Different Species by ELISA

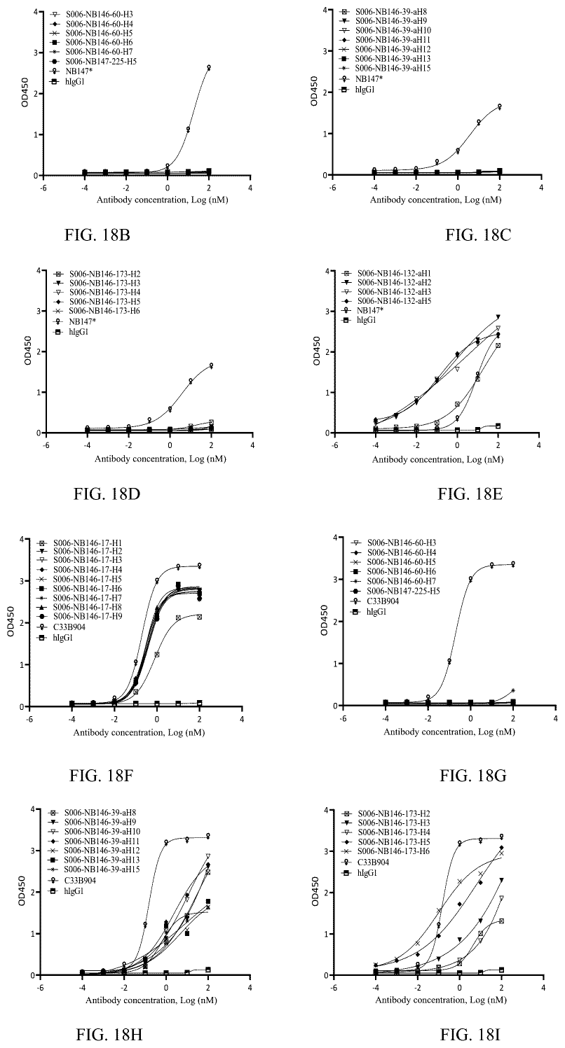

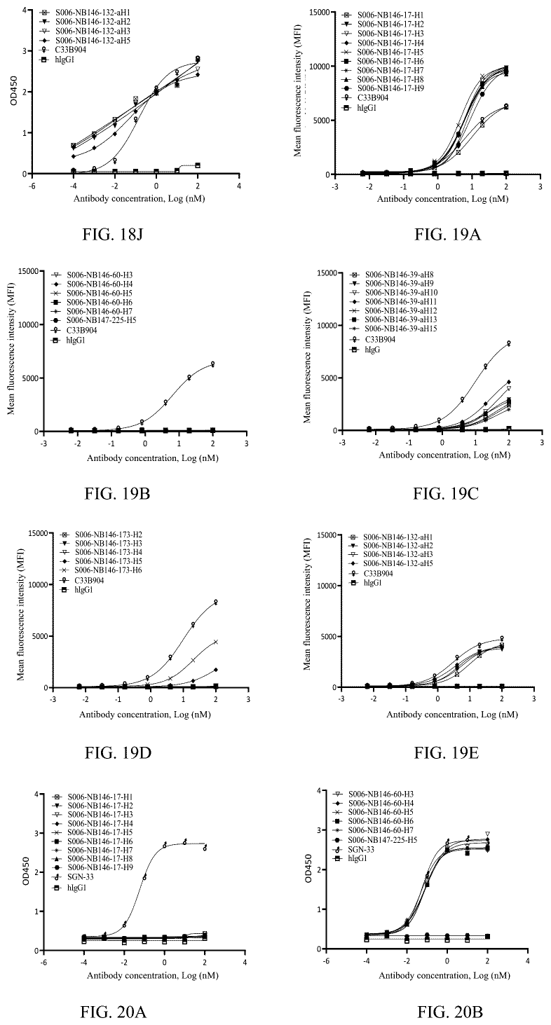

ELISA was performed according to the method in Example 5.1. The assay results for the binding of the VHH-Fc humanized antibodies to murine CD33 by ELISA are shown in FIGS. 18A-18E, and the assay results for the binding of the VHH-Fc humanized antibodies to monkey CD33 by ELISA are shown in FIGS. 18F-18J. Except for the humanized antibodies of S006-NB146-132, other humanized antibodies substantially did not bind to murine CD33; and humanized antibodies other than S006-NB146-60 and S006-NB147-225 bound to monkey CD33.

### 11.2 Assay on Binding of VHH-Fc Humanized Antibodies to Monkey CD33 Recombination Cell Line by FACS

HEK293T-monkey CD33 cell line was subjected to the FACS assay and data analysis according to the method in Example 5.2. The analysis results are shown in FIGS. 19A-19E, which show that, except for humanized antibodies of S006-NB146-60 and S006-NB147-225, the remaining humanized antibodies had binding activity with HEK293T-monkey CD33 cells.

In addition, all humanized antibodies were assayed to have no binding activity with HEK293T cells which served as a negative control.

### Example 12: Assay on Affinity of CD33 Antibodies

### 12.1 Assay on Affinity of VHH-Fc Humanized Antibodies for Human CD33-his Protein

The association rate (Kon), dissociation rate (Koff), and binding affinity (KD) of the VHH-Fc humanized antibodies with the human CD33-his protein were assayed with reference to the method described in Example 7.1; as shown in Table 42, only part of the humanized antibodies were assayed, wherein the antibodies C33B904 and SGN-33 were used as positive controls. As shown in Table 42, the affinity of the assayed VHH-Fc humanized antibodies for human CD33 was superior to 1.61E-07 M.

TABLE 42
Assay on affinity of VHH-Fc antibodies
for human CD33 by SPR (biacore)

| Antibody name | Kon (1/Ms) | Koff (1/s) | KD (M) |
|---|---|---|---|
| S006-NB146-17 | 1.18E+05 | 2.34E−05 | 1.98E−10 |
| S006-NB146-17-H1 | 2.34E+04 | 3.76E−03 | 1.61E−07 |
| S006-NB146-17-H2 | 4.87E+04 | 1.03E−04 | 2.11E−09 |
| S006-NB146-17-H3 | 7.30E+04 | 9.69E−05 | 1.33E−09 |
| S006-NB146-17-H4 | 7.83E+04 | 6.68E−05 | 8.53E−10 |
| S006-NB146-17-H5 | 7.73E+04 | 7.11E−05 | 9.20E−10 |
| S006-NB146-17-H6 | 7.49E+04 | 4.47E−05 | 5.98E−10 |
| S006-NB146-17-H7 | 7.16E+04 | 7.60E−05 | 1.06E−09 |
| S006-NB146-17-H8 | 7.17E+04 | 7.69E−05 | 1.07E−09 |
| S006-NB146-17-H9 | 7.32E+04 | 8.86E−05 | 1.21E−09 |
| S006-NB146-60 | 1.25E+05 | 1.47E−04 | 1.18E−09 |
| S006-NB146-60-H3 | 1.86E+04 | 5.07E−04 | 2.72E−08 |
| S006-NB146-60-H4 | 4.32E+04 | 1.51E−04 | 3.49E−09 |
| S006-NB146-60-H5 | 1.48E+04 | 8.17E−04 | 5.53E−08 |
| S006-NB146-60-H6 | 1.17E+04 | 8.15E−04 | 6.98E−08 |
| S006-NB146-60-H7 | 1.85E+04 | 4.25E−04 | 2.29E−08 |
| S006-NB147-225 | 8.19E+04 | 2.33E−04 | 2.84E−09 |
| S006-NB147-225-H5 | 3.59E+04 | 4.98E−04 | 1.39E−08 |
| S006-NB146-132 | 1.31E+05 | 6.96E−05 | 5.31B−10 |
| S006-NB146-132-aH1 | 3.17E+05 | 1.42E−03 | 4.50E−09 |
| S006-NB146-132-aH2 | 5.37E+05 | 2.80E−04 | 5.21E−10 |
| S006-NB146-132-aH3 | 4.77E+05 | 2.94E−04 | 6.17E−10 |
| S006-NB146-132-aH5 | 6.88E+05 | 2.34E−04 | 3.41E−10 |
| S006-NB146-39-aH8 | 2.04E+05 | 9.00E−04 | 4.42E−09 |
| S006-NB146-39-aH9 | 2.55E+05 | 1.34E−03 | 5.25E−09 |
| S006-NB146-39-aH10 | 1.99E+05 | 7.85E−05 | 3.95E−10 |
| S006-NB146-39-aH11 | 2.80E+05 | 1.51E−04 | 5.38E−10 |
| S006-NB146-39-aH12 | 2.08E+05 | 1.50E−03 | 7.21E−09 |
| S006-NB146-39-aH13 | 2.31E+05 | 1.95E−03 | 8.43E−09 |
| S006-NB146-39-aH15 | 1.58E+05 | 9.10E−04 | 5.77E−09 |
| S006-NB146-173 | 1.85E+05 | 1.79E−03 | 9.68E−09 |
| S006-NB146-173-H5 | 9.99E+04 | 6.11E−03 | 6.12E−08 |
| S006-NB146-173-H6 | 2.14E+05 | 1.63E−03 | 7.60E−09 |
| C33B904 | 8.28E+05 | 6.54E−05 | 7.90E−11 |
| SGN-33 | 7.02E+05 | 5.57E−03 | 7.93E−09 |

### 12.2 Assay on Affinity of VHH-Fc Antibodies for Monkey CD33-his Protein

The assay on affinity of VHH-Fc antibodies for monkey CD33 protein (Sino Biological, Cat. No. 50712-M08H) was performed according to the method in Example 7.2. As shown in Table 43, the affinity of VHH-Fc humanized antibodies for monkey CD33 was superior to 1.0-07 M.

TABLE 43
Assay on affinity of VHH-Fe antibodies
for monkey CD33 by SPR (biacore)

| Antibody name | Kon (1/Ms) | Koff (1/s) | KD (M) |
|---|---|---|---|
| S006-NB146-39-aH8 | 3.71E+05 | 1.77E−02 | 4.78E−08 |
| S006-NB146-39-aH9 | 4.34E+05 | 1.85E−02 | 4.26E−08 |
| S006-NB146-39-aH10 | 3.65E+05 | 8.29E−04 | 2.27E−09 |
| S006-NB146-39-aH11 | 5.34E+05 | 1.45E−03 | 2.72E−09 |
| S006-NB146-39-aH12 | 3.77E+05 | 2.65E−02 | 7.02E−08 |
| S006-NB146-39-aH13 | 3.83E+05 | 2.35E−02 | 6.14E−08 |
| S006-NB146-39-aH15 | 3.08E+05 | 1.71E−02 | 5.57E−08 |
| S006-NB146-173-H5 | 1.52E+05 | 1.44E−02 | 9.51E−08 |
| S006-NB146-173-H6 | 3.23E+05 | 5.20E−03 | 1.61E−08 |
| C33B904 | 1.11E+06 | 6.26E−04 | 5.65E−10 |

### Example 13: Identification of Antigen-binding Regions of Antibodies

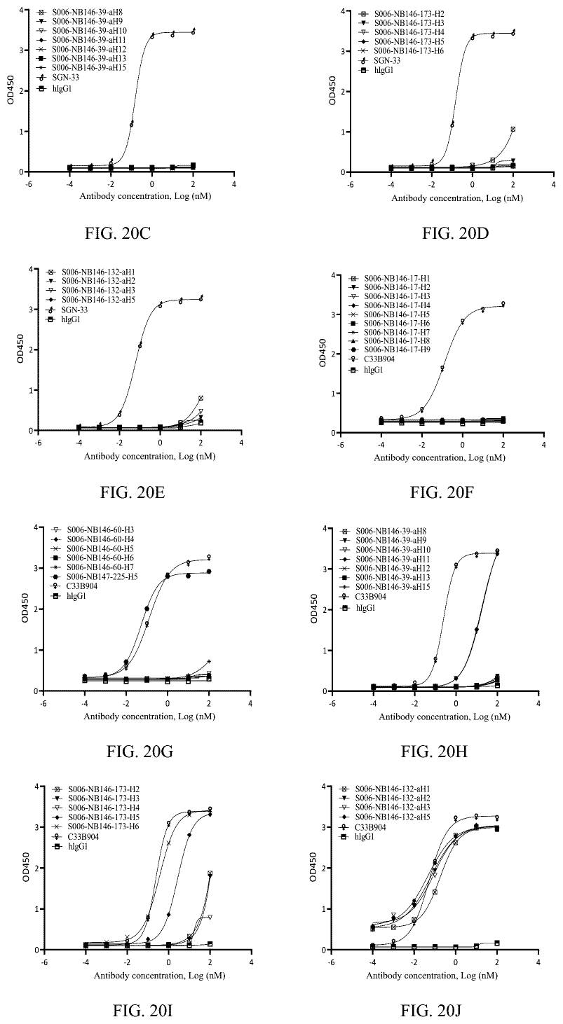

Plates were coated with human CD33-V-his (membrane distal end) and human CD33-C2-his (membrane proximal end), respectively, according to the ELISA method in Example 8. The VHH humanized antibodies were classified based on the membrane distal end and the membrane proximal end, as shown in FIGS. 20A-20J and Table 44, which allowed the classification of the VHH humanized antibodies into the following classes:

The antibodies obtained after humanization of molecule S006-NB146-17 could neither bind to the human CD33-C2-his protein nor bind to the human CD33-V-his protein, and therefore belong to spatial conformation, being consistent with the parent clone epitope. The antibodies obtained after humanization of molecule S006-NB146-60 could bind to the human CD33-V-his protein, so the antigen-binding epitopes thereof belong to V domain. The antibody obtained after humanization of molecule S006-NB147-225 could bind to the human CD33-C2-his protein, so the antigen-binding epitope thereof belongs to C2 domain. Among the antibodies obtained after humanization of molecule S006-NB146-39, only S006-NB146-39-aH10 and S006-NB146-39-aH11 could bind to the human CD33-C2-his protein, so the antigen-binding epitopes of these two belong to C2 domain. Except for S006-NB146-173-H2, the remaining antibodies obtained after humanization of molecule S006-NB146-173 belong to C2 domain, being consistent with the parent clone. The antibody obtained after humanization of molecule S006-NB146-132 could bind to the human CD33-C2-his protein, so the antigen-binding epitopes thereof belong to C2 domain.

TABLE 44
Classification of membrane distal end and
membrane proximal end epitopes for
VHH antibodies by ELISA

| | Binding region | |
|---|---|---|
| Antibody name | V-domain | C2-domain |
| S006-NB146-17-H1 | − | − |
| S006-NB146-17-H2 | − | − |
| S006-NB146-17-H3 | − | − |
| S006-NB146-17-H4 | − | − |
| S006-NB146-17-H5 | − | − |
| S006-NB146-17-H6 | − | − |
| S006-NB146-17-H7 | − | − |
| S006-NB146-17-H8 | − | − |
| S006-NB146-17-H9 | − | − |
| S006-NB146-60-H3 | + | − |
| S006-NB146-60-H4 | + | − |
| S006-NB146-60-H5 | + | − |
| S006-NB146-60-H6 | + | − |
| S006-NB146-60-H7 | + | − |
| S006-NB147-225-H5 | − | + |
| S006-NB146-39-aH8 | − | − |
| S006-NB146-39-aH9 | − | − |
| S006-NB146-39-aH10 | − | + |
| S006-NB146-39-aH11 | − | + |
| S006-NB146-39-aH12 | − | − |
| S006-NB146-39-aH13 | − | − |
| S006-NB146-39-aH15 | − | − |
| S006-NB146-173-H2 | + | + |
| S006-NB146-173-H3 | − | + |
| S006-NB146-173-H4 | − | + |
| S006-NB146-173-HS | − | + |
| S006-NB146-173-H6 | − | + |
| S006-NB146-132-aH1 | − | + |
| S006-NB146-132-aH2 | − | + |
| S006-NB146-132-aH3 | − | + |
| S006-NB146-132-aH5 | − | + |

### Reference to a “Sequence Listing,” a Table, or a Computer Program Listing Appendix Submitted as an Ascii Text File

The material in the ASCII text file, named “COTAL-68801-Sequence-Listing_ST25.txt”, created Aug. 2, 2023, file size of 110.592 bytes, is hereby incorporated by reference.

1. An antibody or an antigen-binding fragment specifically binding to CD33, comprising: a CDR1, a CDR2, and a CDR3, wherein the sequence of the CDR1, the CDR2, and the CDR3 are selected from any one of the following sequence combinations:
according to a universal analysis method of KABAT
(1) CDR1: SEQ ID NO: 53, CDR2: SEQ ID NO: 54 or 230, CDR3: SEQ ID NO: 55;
(2) CDR1: SEQ ID NO: 35, CDR2: SEQ ID NO: 36, CDR3: SEQ ID NO: 37; or
according to a universal analysis method of Chothia
(1) CDR1: SEQ ID NO: 107, CDR2: SEQ ID NO: 108, CDR3: SEQ ID NO: 109:
(2) CDR1: SEQ ID NO: 89, CDR2: SEQ ID NO: 90, CDR3: SEQ ID NO: 91; or
according to a universal analysis method of IMGT
(1) CDR1: SEQ ID NO: 161, CDR2: SEQ ID NO: 162, CDR3: SEQ ID NO: 163:
(2) CDR1: SEQ ID NO: 143 or 226 or 227, CDR2: SEQ ID NO: 144, CDR3: SEO ID NO: 145; or
a sequence compared with the above sequence combinations, have the substitutions with conserved amino acids.

2. The antibody or the antigen-binding fragment according to claim 1, having a VHH domain selected from:
(1) SEQ ID NO: 19 or 217:
(2) SEQ ID NO: 13 or 197; or
(3) a sequence with 1 to 3 amino acid substitutions compared with the sequences (1) or (2), wherein the substitutions are conservative amino acid substitutions.

3. The antibody or the antigen-binding fragment according to claim 1, comprising sequences having at least 95%, identity to the CDR1, the CDR2, and/or the CDR3.

4. The antibody or the antigen-binding fragment according to claim 2, wherein the antibody or the antigen-binding fragment comprises a sequence having at least 90% identity to the sequence set forth in any one of SEQ ID NOs: 19, 217, 13 or 197.

5. The antibody or the antigen-binding fragment according to claim 1, wherein the antibody or the antigen-binding fragment binds to human CD33 with a dissociation constant (KD) of not greater than 100 nM, and binds to monkey CD33 with a KD of not greater than 100 nM.

6. The antibody or the antigen-binding fragment according to claim 1, wherein the antibody or the antigen-binding fragment comprises or does not comprise an antibody heavy chain constant region; optionally, the antibody heavy chain constant region may be selected from human, Vicugna pacos, mouse, rat, rabbit, and sheep; optionally, the antibody heavy chain constant region may be selected from IgG, IgM, IgA, IgE, and IgD, and the IgG may be selected from IgG1, IgG2, IgG3, and IgG4; optionally, the heavy chain constant region may be selected from an Fc region, a CH3 region, a heavy chain constant region without a CH1 fragment, and an intact heavy chain constant region.

7. The antibody or the antigen-binding fragment according to claim 6, wherein the heavy chain constant region is a human Fc region, having an amino acid sequence set forth in SEQ ID NO: 191.

8. The antibody or the antigen-binding fragment according to claim 6, wherein the antibody or the antigen-binding fragment is a heavy chain antibody.

9. The antibody or the antigen-binding fragment according to claim 1, wherein the antibody or the antigen-binding fragment is:
(1) a chimeric antibody or a fragment thereof;
(2) a humanized antibody or a fragment thereof; or
(3) a full human antibody or a fragment thereof.

10. The antibody or the antigen-binding fragment according to claim 1, wherein the antibody or the antigen-binding fragment is further conjugated to a therapeutic agent or a tracer or the antigen-binding fragment is further conjugated to another functional molecule, wherein the functional molecule may be selected from one or more of a signal peptide, a protein tag, and a cytokine.

11. (canceled)

12. A multispecific antibody comprising the antibody or the antigen-binding fragment according to claim 1, wherein the multispecific antibody further comprises an antibody or an antigen-binding fragment specifically binding to an antigen other than CD33 or binding to an additional epitope of CD33, wherein the antigen other than CD33 may be selected from the group consisting of: CD3; CD16; CS32B; PD-1; PD-2; PD-L1; NKG2D; CD19; CD20; CD40; CD47; 4-1BB; CD137; EGFR; EGFRvIII; TNF-alpha; MSLN; HER2; HER3; HSA; CD5; CD27; EphA2; EpCAM: MUC1; MUC16; CEA; Claudin18.2; folate receptor; Claudin6; WT1; NY-ESO-1; MAGE3; ASGPR1; and CDH16.

13-14. (canceled)

15. A chimeric antigen receptor (CAR) comprising an extracellular antigen-binding domain, a transmembrane domain, and an intracellular signaling domain, wherein the extracellular antigen-binding domain comprises the antibody or the antigen-binding fragment according to claim 1.

16. An immune effector cell that expresses, or comprises a nucleic acid fragment encoding, a chimeric antigen receptor (CAR), comprising an extracellular antigen-binding domain, a transmembrane domain, and an intracellular signaling domain, wherein the extracellular antigen-binding domain comprises the antibody or the antigen-binding fragment according to claim 1, wherein the immune effector cell is selected from the group consisting of a T cell, a natural killer (NK) cell, a natural killer T (NKT) cell, a double negative T (DNT) cell, a monocyte, a macrophage, a dendritic cell, and a mast cell.

17. An isolated nucleic acid fragment, wherein the nucleic acid fragment encodes the antibody or the antigen-binding fragment according to claim 1.

18. A vector comprising the nucleic acid fragment according to claim 17.

19. A host cell comprising the vector according to claim 18.

20-21. (canceled)

22. A pharmaceutical composition comprising the antibody or the antigen-binding fragment according to claim 1; and, optionally, the pharmaceutical composition further comprises a pharmaceutically acceptable carrier, diluent, or adjuvant; and, optionally, the pharmaceutical composition further comprises an additional antineoplastic agent.

23. (canceled)

24. A method for preventing and/or treating a tumor, comprising: administering to a patient in need thereof an effective amount of the antibody or the antigen-binding fragment according to claim 1, wherein the tumor is selected from myelodysplastic syndrome (MDS), acute myelogenous leukemia (AML), chronic myelogenous leukemia (CML), and promyelocytic leukemia (PML).

25-27. (canceled)


SEQUENCE LISTING (231 sequences)

SEQ ID NO: 1 - protein, 471 aa, Artificial Sequence
  Synthetic sequence
QVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSSGGGGSGGGGSGGGGSDIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIKEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 449 aa, Artificial Sequence
  Synthetic sequence
EVQLVESGGGLVQPGRSLRLSCAASGFTFDDYAMHWVRQAPGKGLEWVSGIGWSGGSIVYADSVKGRFTISRDNAKNSLYLQMNSLRAEDTALYYCAKDSPYGDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 220 aa, Artificial Sequence
  Synthetic sequence
DIVMTQSPDSLAVSLGERATINCKSSQTVFYSSNNKNYLAWYQQKPGQPPKLLISWASTRKSGVPDRFSGSGSGTDFTLTVSSLQAEDVAVYYCQHYYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 129 aa, Artificial Sequence
  Synthetic sequence
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRGS

SEQ ID NO: 5 - protein, 126 aa, Artificial Sequence
  Synthetic sequence
VHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGS

SEQ ID NO: 6 - DNA, 18 bp, Artificial Sequence
  Synthetic sequence
cttggtggtcctggctgc

SEQ ID NO: 7 - DNA, 23 bp, Artificial Sequence
  Synthetic sequence
ggtacgtgctgttgaactgttcc

SEQ ID NO: 8 - DNA, 47 bp, Artificial Sequence
  Synthetic sequence
catgccatgactgtggcccaggcggcccagktgcagctcgtggagtc

SEQ ID NO: 9 - DNA, 51 bp, Artificial Sequence
  Synthetic sequence
catgccatgactcgcggccggcctggccatgggggtcttcgctgtggtgcg

SEQ ID NO: 10 - DNA, 52 bp, Artificial Sequence
  Synthetic sequence
catgccatgactcgcggccggcctggccgtcttgtggttttggtgtcttggg

SEQ ID NO: 11 - protein, 119 aa, Artificial Sequence
  Synthetic sequence
EVQVVESGGGLVQAGGSLTLSCAASGNIENIQVMGWYRQAPGSQRALVAQINLSGLESTNYLDSVKERFTISRDYTKNAVYLRMNRLEPEDTAVYYCNARSYYGQYDYWGQGTQVTVSS

SEQ ID NO: 12 - protein, 125 aa, Artificial Sequence
  Synthetic sequence
EVQVVESGGGLVQSGGSLRLSCAASGAIFTSNAMDYAMGWYRQVSGKQREWVATITSDDDTNYGDSAKGRFTISRDSAKNTAYLQMNSLKPEDTAVYYCFANVRSDRSTLYRNYWGQGTQVTVSS

SEQ ID NO: 13 - protein, 125 aa, Artificial Sequence
  Synthetic sequence
QVQLVESGGGLVQSGGSLRLSCAASGSIFTSDDMDYAMGWYRQASGKQREWVATITSDEDTNYGDSVKGRFTISRDSAKNTAFLQMNTLKPEDTAVYYCFANVRSDRSSLYRNYWGQGTQVTVSS

SEQ ID NO: 14 - protein, 113 aa, Artificial Sequence
  Synthetic sequence
EVQLVESGGGSVQAGGSLRLSCGTPQSWFRLSSYVMNWYRQAPGKRRELVAEITQDDTTNYTPSVKGRFAISRDNANNTVHLQMNSLKPEDTAVYYCNTIQIWGQGTQVTVSS

SEQ ID NO: 15 - protein, 130 aa, Artificial Sequence
  Synthetic sequence
EVQLVESGGGLVQPGESLRLSCTAPGIILDYYPIGWFRQAPGKEREGVAFITNSDDSTIYTNYADSVKGRFTISREKNTVYLQMNSLKPEDTALYYCAARSTYIVGRNENALNSSDYDYWGQGTQVTVSS

SEQ ID NO: 16 - protein, 125 aa, Artificial Sequence
  Synthetic sequence
QVQLVESGGGLVQSGGSLRLSCAASGSIFTSNDMDYAMGWYRQVSGKQREWVATITSDEDTNYGDSVKGRFTISRDSAKNTAYLQMNSLKPEDTAVYYCFANLRSDRSTLYRNYWGQGTQVTVSS

SEQ ID NO: 17 - protein, 130 aa, Artificial Sequence
  Synthetic sequence
QLQLVESGGGLVQPGGSLRLSCTAPGIVLDYYPIGWFRQAPGKEREGVSFITNSDDSTIYTNYADSVKGRFTISREKNTVYLQMNSLKPEDTAVYYCAARSTYIVGRNENALNASDYDYWGQGTQVTVSS

SEQ ID NO: 18 - protein, 121 aa, Artificial Sequence
  Synthetic sequence
EVQLVESGGGLVQAGGSLRLSCVASRSTSSLSLMSWHRQAPGEQRELVASITSGGKANYVDSVKDRFIMTRDDAKNTVYLQMNSLKPEDTAVYYCHAYDRAGTASGFGAAWGQGTQVTVSS

SEQ ID NO: 19 - protein, 120 aa, Artificial Sequence
  Synthetic sequence
QVQLVESGGGLVQAGGSLRLSCAASGGTFVINAAWYHQAPGKQRELVANISYGGSTNYADRVAGRFAISRDNAKNTVYLQMNSLKPEDTGVYYCRLDPGLALNNRPLYYWGQGTQVTVSS

SEQ ID NO: 20 - protein, 118 aa, Artificial Sequence
  Synthetic sequence
EVQVVESGGGSVQPGGSLRLSCVASGFTFSNYAMSWVRQTPKKGLEWVAEINSGGDNTYYAYFVKGRFTISRDNAKNTLYLQMNSLKPEDTGTYYCAREEENAGTILQGQGTQVTVSS

SEQ ID NO: 21 - protein, 119 aa, Artificial Sequence
  Synthetic sequence
EVQLVESGGGLVPAGGSLRLSCAASGSIANIQVMGWYRQAPGSQRALVAQITVTGDRATNYADSVKGRFTISRDNAKNAVYLSINPLKSEDTGVYYCNARSYYGQHNYWGQGTQVTVSS

SEQ ID NO: 22 - protein, 121 aa, Artificial Sequence
  Synthetic sequence
QLQLVESGGGLVQAGGSLRLSCKASGVTFSINALGWYRQAPGKQREMVAVISSGGSTNYVDSVKGRFTISRDNAKNTVYLQMNSLKPEDTAVYYCNADVVGSSWSPEYDYWGQGTQVTVST

SEQ ID NO: 23 - protein, 121 aa, Artificial Sequence
  Synthetic sequence
EVQVVESGGGLVQAGGSLSLSCAASGSIFTINIMGWYRQAPGKQRELVAQINRVGDTNYPNSMAERFTISRDNAKNTMYLQMNTLKPEDTAVYYCNADIVESGRYSGKDYWGKGTLVTVSL

SEQ ID NO: 24 - protein, 130 aa, Artificial Sequence
  Synthetic sequence
QLQLVESGGGLVQPGGSLRLSCAAPGIILDYYPIGWFRQAPGKEREGVSYITSSDDSTVYTYYTDSVKGRFTISRGKNTVYLQMNSLKPEDTAVYYCAARSTYIVGRNYHTLNSSDFDYWGQGTQVTVSS

SEQ ID NO: 25 - protein, 121 aa, Artificial Sequence
  Synthetic sequence
QLQLVESGGGLVQAGGSLRLSCAASGNSLGVYNLGWYRQAPGEQRELVATINSRGDTNYVNSVAGRFTISRVNARNTENTVYLQMNRLQPEDTAVYYCIAQRGWGREYDIWGPGTQVTVSS

SEQ ID NO: 26 - protein, 117 aa, Artificial Sequence
  Synthetic sequence
QVQLVESGGGLVQAGGSLRLSCAASGSTLSLHSMGWYRQAAGRQRELVAGISRDGDSNYLSSLAGRFFISRDGTKNTVYLQMTSLKPEDTAVYYCNARTSPWSAALWGQGTQVTVSS

SEQ ID NO: 27 - protein, 115 aa, Artificial Sequence
  Synthetic sequence
QVQLVESGGGLVQAGGSLRLSCAASGSIFSMGWYRQAPGKQRELVAAIANGGTTTNYANSVKDRFTISRDNAKNTVYLQMNSLRPEDTAVYYCYAKRSWDSSSYWGQGTQVTVSS

SEQ ID NO: 28 - protein, 128 aa, Artificial Sequence
  Synthetic sequence
QLQLVESGGGMVQPGGSLRLSCAASGFTLDYYAIGWFRQAPGKEREGVSCINSGDSSTYYADSVKGRFTISRDNAENTVYLQMNSLKPEDTAVYYCATVDTLIVAGNLEECRYDMDYWGKGTLVTVSS

SEQ ID NO: 29 - protein, 5 aa, Artificial Sequence
  Synthetic sequence
IQVMG

SEQ ID NO: 30 - protein, 18 aa, Artificial Sequence
  Synthetic sequence
QINLSGLESTNYLDSVKE

SEQ ID NO: 31 - protein, 9 aa, Artificial Sequence
  Synthetic sequence
RSYYGQYDY

SEQ ID NO: 32 - protein, 9 aa, Artificial Sequence
  Synthetic sequence
SNAMDYAMG

SEQ ID NO: 33 - protein, 16 aa, Artificial Sequence
  Synthetic sequence
TITSDDDTNYGDSAKG

SEQ ID NO: 34 - protein, 13 aa, Artificial Sequence
  Synthetic sequence
NVRSDRSTLYRNY

SEQ ID NO: 35 - protein, 9 aa, Artificial Sequence
  Synthetic sequence
SDDMDYAMG

SEQ ID NO: 36 - protein, 16 aa, Artificial Sequence
  Synthetic sequence
TITSDEDTNYGDSVKG

SEQ ID NO: 37 - protein, 13 aa, Artificial Sequence
  Synthetic sequence
NVRSDRSSLYRNY

SEQ ID NO: 38 - protein, 7 aa, Artificial Sequence
  Synthetic sequence
LSSYVMN

SEQ ID NO: 39 - protein, 16 aa, Artificial Sequence
  Synthetic sequence
EITQDDTTNYTPSVKG

SEQ ID NO: 40 - protein, 3 aa, Artificial Sequence
  Synthetic sequence
IQI

SEQ ID NO: 41 - protein, 5 aa, Artificial Sequence
  Synthetic sequence
YYPIG

SEQ ID NO: 42 - protein, 20 aa, Artificial Sequence
  Synthetic sequence
FITNSDDSTIYTNYADSVKG

SEQ ID NO: 43 - protein, 20 aa, Artificial Sequence
  Synthetic sequence
RSTYIVGRNENALNSSDYDY

SEQ ID NO: 44 - protein, 9 aa, Artificial Sequence
  Synthetic sequence
SNDMDYAMG

SEQ ID NO: 45 - protein, 16 aa, Artificial Sequence
  Synthetic sequence
TITSDEDTNYGDSVKG

SEQ ID NO: 46 - protein, 13 aa, Artificial Sequence
  Synthetic sequence
NLRSDRSTLYRNY

SEQ ID NO: 47 - protein, 5 aa, Artificial Sequence
  Synthetic sequence
YYPIG

SEQ ID NO: 48 - protein, 20 aa, Artificial Sequence
  Synthetic sequence
FITNSDDSTIYTNYADSVKG

SEQ ID NO: 49 - protein, 20 aa, Artificial Sequence
  Synthetic sequence
RSTYIVGRNENALNASDYDY

SEQ ID NO: 50 - protein, 5 aa, Artificial Sequence
  Synthetic sequence
LSLMS

SEQ ID NO: 51 - protein, 16 aa, Artificial Sequence
  Synthetic sequence
SITSGGKANYVDSVKD

SEQ ID NO: 52 - protein, 13 aa, Artificial Sequence
  Synthetic sequence
YDRAGTASGFGAA

SEQ ID NO: 53 - protein, 4 aa, Artificial Sequence
  Synthetic sequence
INAA

SEQ ID NO: 54 - protein, 16 aa, Artificial Sequence
  Synthetic sequence
NISYGGSTNYADRVAG

SEQ ID NO: 55 - protein, 13 aa, Artificial Sequence
  Synthetic sequence
DPGLALNNRPLYY

SEQ ID NO: 56 - protein, 5 aa, Artificial Sequence
  Synthetic sequence
NYAMS

SEQ ID NO: 57 - protein, 17 aa, Artificial Sequence
  Synthetic sequence
EINSGGDNTYYAYFVKG

SEQ ID NO: 58 - protein, 9 aa, Artificial Sequence
  Synthetic sequence
EEENAGTIL

SEQ ID NO: 59 - protein, 5 aa, Artificial Sequence
  Synthetic sequence
IQVMG

SEQ ID NO: 60 - protein, 18 aa, Artificial Sequence
  Synthetic sequence
QITVTGDRATNYADSVKG

SEQ ID NO: 61 - protein, 9 aa, Artificial Sequence
  Synthetic sequence
RSYYGQHNY

SEQ ID NO: 62 - protein, 5 aa, Artificial Sequence
  Synthetic sequence
INALG

SEQ ID NO: 63 - protein, 16 aa, Artificial Sequence
  Synthetic sequence
VISSGGSTNYVDSVKG

SEQ ID NO: 64 - protein, 13 aa, Artificial Sequence
  Synthetic sequence
DVVGSSWSPEYDY

SEQ ID NO: 65 - protein, 5 aa, Artificial Sequence
  Synthetic sequence
INIMG

SEQ ID NO: 66 - protein, 16 aa, Artificial Sequence
  Synthetic sequence
QINRVGDTNYPNSMAE

SEQ ID NO: 67 - protein, 13 aa, Artificial Sequence
  Synthetic sequence
DIVESGRYSGKDY

SEQ ID NO: 68 - protein, 5 aa, Artificial Sequence
  Synthetic sequence
YYPIG

SEQ ID NO: 69 - protein, 20 aa, Artificial Sequence
  Synthetic sequence
YITSSDDSTVYTYYTDSVKG

SEQ ID NO: 70 - protein, 20 aa, Artificial Sequence
  Synthetic sequence
RSTYIVGRNYHTLNSSDFDY

SEQ ID NO: 71 - protein, 5 aa, Artificial Sequence
  Synthetic sequence
VYNLG

SEQ ID NO: 72 - protein, 16 aa, Artificial Sequence
  Synthetic sequence
TINSRGDTNYVNSVAG

SEQ ID NO: 73 - protein, 10 aa, Artificial Sequence
  Synthetic sequence
QRGWGREYDI

SEQ ID NO: 74 - protein, 5 aa, Artificial Sequence
  Synthetic sequence
LHSMG

SEQ ID NO: 75 - protein, 16 aa, Artificial Sequence
  Synthetic sequence
GISRDGDSNYLSSLAG

SEQ ID NO: 76 - protein, 9 aa, Artificial Sequence
  Synthetic sequence
RTSPWSAAL

SEQ ID NO: 77 - protein, 2 aa, Artificial Sequence
  Synthetic sequence
MG

SEQ ID NO: 78 - protein, 17 aa, Artificial Sequence
  Synthetic sequence
AIANGGTTTNYANSVKD

SEQ ID NO: 79 - protein, 9 aa, Artificial Sequence
  Synthetic sequence
KRSWDSSSY

SEQ ID NO: 80 - protein, 5 aa, Artificial Sequence
  Synthetic sequence
YYAIG

SEQ ID NO: 81 - protein, 17 aa, Artificial Sequence
  Synthetic sequence
CINSGDSSTYYADSVKG

SEQ ID NO: 82 - protein, 19 aa, Artificial Sequence
  Synthetic sequence
VDTLIVAGNLEECRYDMDY

SEQ ID NO: 83 - protein, 7 aa, Artificial Sequence
  Synthetic sequence
GNIENIQ

SEQ ID NO: 84 - protein, 7 aa, Artificial Sequence
  Synthetic sequence
NLSGLES

SEQ ID NO: 85 - protein, 9 aa, Artificial Sequence
  Synthetic sequence
RSYYGQYDY

SEQ ID NO: 86 - protein, 11 aa, Artificial Sequence
  Synthetic sequence
GAIFTSNAMDY

SEQ ID NO: 87 - protein, 5 aa, Artificial Sequence
  Synthetic sequence
TSDDD

SEQ ID NO: 88 - protein, 13 aa, Artificial Sequence
  Synthetic sequence
NVRSDRSTLYRNY

SEQ ID NO: 89 - protein, 11 aa, Artificial Sequence
  Synthetic sequence
GSIFTSDDMDY

SEQ ID NO: 90 - protein, 5 aa, Artificial Sequence
  Synthetic sequence
TSDED

SEQ ID NO: 91 - protein, 13 aa, Artificial Sequence
  Synthetic sequence
NVRSDRSSLYRNY

SEQ ID NO: 92 - protein, 9 aa, Artificial Sequence
  Synthetic sequence
QSWFRLSSY

SEQ ID NO: 93 - protein, 5 aa, Artificial Sequence
  Synthetic sequence
TQDDT

SEQ ID NO: 94 - protein, 3 aa, Artificial Sequence
  Synthetic sequence
IQI

SEQ ID NO: 95 - protein, 7 aa, Artificial Sequence
  Synthetic sequence
GIILDYY

SEQ ID NO: 96 - protein, 9 aa, Artificial Sequence
  Synthetic sequence
TNSDDSTIY

SEQ ID NO: 97 - protein, 20 aa, Artificial Sequence
  Synthetic sequence
RSTYIVGRNENALNSSDYDY

SEQ ID NO: 98 - protein, 11 aa, Artificial Sequence
  Synthetic sequence
GSIFTSNDMDY

SEQ ID NO: 99 - protein, 5 aa, Artificial Sequence
  Synthetic sequence
TSDED

SEQ ID NO: 100 - protein, 13 aa, Artificial Sequence
  Synthetic sequence
NLRSDRSTLYRNY

SEQ ID NO: 101 - protein, 7 aa, Artificial Sequence
  Synthetic sequence
GIVLDYY

SEQ ID NO: 102 - protein, 9 aa, Artificial Sequence
  Synthetic sequence
TNSDDSTIY

SEQ ID NO: 103 - protein, 20 aa, Artificial Sequence
  Synthetic sequence
RSTYIVGRNENALNASDYDY

SEQ ID NO: 104 - protein, 7 aa, Artificial Sequence
  Synthetic sequence
RSTSSLS

SEQ ID NO: 105 - protein, 5 aa, Artificial Sequence
  Synthetic sequence
TSGGK

SEQ ID NO: 106 - protein, 13 aa, Artificial Sequence
  Synthetic sequence
YDRAGTASGFGAA

SEQ ID NO: 107 - protein, 6 aa, Artificial Sequence
  Synthetic sequence
GGTFVI

SEQ ID NO: 108 - protein, 5 aa, Artificial Sequence
  Synthetic sequence
SYGGS

SEQ ID NO: 109 - protein, 13 aa, Artificial Sequence
  Synthetic sequence
DPGLALNNRPLYY

SEQ ID NO: 110 - protein, 7 aa, Artificial Sequence
  Synthetic sequence
GFTFSNY

SEQ ID NO: 111 - protein, 6 aa, Artificial Sequence
  Synthetic sequence
NSGGDN

SEQ ID NO: 112 - protein, 9 aa, Artificial Sequence
  Synthetic sequence
EEENAGTIL

SEQ ID NO: 113 - protein, 7 aa, Artificial Sequence
  Synthetic sequence
GSIANIQ

SEQ ID NO: 114 - protein, 7 aa, Artificial Sequence
  Synthetic sequence
TVTGDRA

SEQ ID NO: 115 - protein, 9 aa, Artificial Sequence
  Synthetic sequence
RSYYGQHNY

SEQ ID NO: 116 - protein, 7 aa, Artificial Sequence
  Synthetic sequence
GVTFSIN

SEQ ID NO: 117 - protein, 5 aa, Artificial Sequence
  Synthetic sequence
SSGGS

SEQ ID NO: 118 - protein, 13 aa, Artificial Sequence
  Synthetic sequence
DVVGSSWSPEYDY

SEQ ID NO: 119 - protein, 7 aa, Artificial Sequence
  Synthetic sequence
GSIFTIN

SEQ ID NO: 120 - protein, 5 aa, Artificial Sequence
  Synthetic sequence
NRVGD

SEQ ID NO: 121 - protein, 13 aa, Artificial Sequence
  Synthetic sequence
DIVESGRYSGKDY

SEQ ID NO: 122 - protein, 7 aa, Artificial Sequence
  Synthetic sequence
GIILDYY

SEQ ID NO: 123 - protein, 9 aa, Artificial Sequence
  Synthetic sequence
TSSDDSTVY

SEQ ID NO: 124 - protein, 20 aa, Artificial Sequence
  Synthetic sequence
RSTYIVGRNYHTLNSSDFDY

SEQ ID NO: 125 - protein, 7 aa, Artificial Sequence
  Synthetic sequence
GNSLGVY

SEQ ID NO: 126 - protein, 5 aa, Artificial Sequence
  Synthetic sequence
NSRGD

SEQ ID NO: 127 - protein, 10 aa, Artificial Sequence
  Synthetic sequence
QRGWGREYDI

SEQ ID NO: 128 - protein, 7 aa, Artificial Sequence
  Synthetic sequence
GSTLSLH

SEQ ID NO: 129 - protein, 5 aa, Artificial Sequence
  Synthetic sequence
SRDGD

SEQ ID NO: 130 - protein, 9 aa, Artificial Sequence
  Synthetic sequence
RTSPWSAAL

SEQ ID NO: 131 - protein, 4 aa, Artificial Sequence
  Synthetic sequence
GSIF

SEQ ID NO: 132 - protein, 6 aa, Artificial Sequence
  Synthetic sequence
ANGGTT

SEQ ID NO: 133 - protein, 9 aa, Artificial Sequence
  Synthetic sequence
KRSWDSSSY

SEQ ID NO: 134 - protein, 7 aa, Artificial Sequence
  Synthetic sequence
GFTLDYY

SEQ ID NO: 135 - protein, 6 aa, Artificial Sequence
  Synthetic sequence
NSGDSS

SEQ ID NO: 136 - protein, 19 aa, Artificial Sequence
  Synthetic sequence
VDTLIVAGNLEECRYDMDY

SEQ ID NO: 137 - protein, 8 aa, Artificial Sequence
  Synthetic sequence
GNIENIQV

SEQ ID NO: 138 - protein, 9 aa, Artificial Sequence
  Synthetic sequence
INLSGLEST

SEQ ID NO: 139 - protein, 11 aa, Artificial Sequence
  Synthetic sequence
NARSYYGQYDY

SEQ ID NO: 140 - protein, 12 aa, Artificial Sequence
  Synthetic sequence
GAIFTSNAMDYA

SEQ ID NO: 141 - protein, 7 aa, Artificial Sequence
  Synthetic sequence
ITSDDDT

SEQ ID NO: 142 - protein, 15 aa, Artificial Sequence
  Synthetic sequence
FANVRSDRSTLYRNY

SEQ ID NO: 143 - protein, 12 aa, Artificial Sequence
  Synthetic sequence
GSIFTSDDMDYA

SEQ ID NO: 144 - protein, 7 aa, Artificial Sequence
  Synthetic sequence
ITSDEDT

SEQ ID NO: 145 - protein, 15 aa, Artificial Sequence
  Synthetic sequence
FANVRSDRSSLYRNY

SEQ ID NO: 146 - protein, 10 aa, Artificial Sequence
  Synthetic sequence
QSWFRLSSYV

SEQ ID NO: 147 - protein, 7 aa, Artificial Sequence
  Synthetic sequence
ITQDDTT

SEQ ID NO: 148 - protein, 5 aa, Artificial Sequence
  Synthetic sequence
NTIQI

SEQ ID NO: 149 - protein, 8 aa, Artificial Sequence
  Synthetic sequence
GIILDYYP

SEQ ID NO: 150 - protein, 11 aa, Artificial Sequence
  Synthetic sequence
ITNSDDSTIYT

SEQ ID NO: 151 - protein, 22 aa, Artificial Sequence
  Synthetic sequence
AARSTYIVGRNENALNSSDYDY

SEQ ID NO: 152 - protein, 12 aa, Artificial Sequence
  Synthetic sequence
GSIFTSNDMDYA

SEQ ID NO: 153 - protein, 7 aa, Artificial Sequence
  Synthetic sequence
ITSDEDT

SEQ ID NO: 154 - protein, 15 aa, Artificial Sequence
  Synthetic sequence
FANLRSDRSTLYRNY

SEQ ID NO: 155 - protein, 8 aa, Artificial Sequence
  Synthetic sequence
GIVLDYYP

SEQ ID NO: 156 - protein, 11 aa, Artificial Sequence
  Synthetic sequence
ITNSDDSTIYT

SEQ ID NO: 157 - protein, 22 aa, Artificial Sequence
  Synthetic sequence
AARSTYIVGRNENALNASDYDY

SEQ ID NO: 158 - protein, 8 aa, Artificial Sequence
  Synthetic sequence
RSTSSLSL

SEQ ID NO: 159 - protein, 7 aa, Artificial Sequence
  Synthetic sequence
ITSGGKA

SEQ ID NO: 160 - protein, 15 aa, Artificial Sequence
  Synthetic sequence
HAYDRAGTASGFGAA

SEQ ID NO: 161 - protein, 7 aa, Artificial Sequence
  Synthetic sequence
GGTFVIN

SEQ ID NO: 162 - protein, 7 aa, Artificial Sequence
  Synthetic sequence
ISYGGST

SEQ ID NO: 163 - protein, 15 aa, Artificial Sequence
  Synthetic sequence
RLDPGLALNNRPLYY

SEQ ID NO: 164 - protein, 8 aa, Artificial Sequence
  Synthetic sequence
GFTFSNYA

SEQ ID NO: 165 - protein, 8 aa, Artificial Sequence
  Synthetic sequence
INSGGDNT

SEQ ID NO: 166 - protein, 11 aa, Artificial Sequence
  Synthetic sequence
AREEENAGTIL

SEQ ID NO: 167 - protein, 8 aa, Artificial Sequence
  Synthetic sequence
GSIANIQV

SEQ ID NO: 168 - protein, 9 aa, Artificial Sequence
  Synthetic sequence
ITVTGDRAT

SEQ ID NO: 169 - protein, 11 aa, Artificial Sequence
  Synthetic sequence
NARSYYGQHNY

SEQ ID NO: 170 - protein, 8 aa, Artificial Sequence
  Synthetic sequence
GVTFSINA

SEQ ID NO: 171 - protein, 7 aa, Artificial Sequence
  Synthetic sequence
ISSGGST

SEQ ID NO: 172 - protein, 15 aa, Artificial Sequence
  Synthetic sequence
NADVVGSSWSPEYDY

SEQ ID NO: 173 - protein, 8 aa, Artificial Sequence
  Synthetic sequence
GSIFTINI

SEQ ID NO: 174 - protein, 7 aa, Artificial Sequence
  Synthetic sequence
INRVGDT

SEQ ID NO: 175 - protein, 15 aa, Artificial Sequence
  Synthetic sequence
NADIVESGRYSGKDY

SEQ ID NO: 176 - protein, 8 aa, Artificial Sequence
  Synthetic sequence
GIILDYYP

SEQ ID NO: 177 - protein, 11 aa, Artificial Sequence
  Synthetic sequence
ITSSDDSTVYT

SEQ ID NO: 178 - protein, 22 aa, Artificial Sequence
  Synthetic sequence
AARSTYIVGRNYHTLNSSDFDY

SEQ ID NO: 179 - protein, 8 aa, Artificial Sequence
  Synthetic sequence
GNSLGVYN

SEQ ID NO: 180 - protein, 7 aa, Artificial Sequence
  Synthetic sequence
INSRGDT

SEQ ID NO: 181 - protein, 12 aa, Artificial Sequence
  Synthetic sequence
IAQRGWGREYDI

SEQ ID NO: 182 - protein, 8 aa, Artificial Sequence
  Synthetic sequence
GSTLSLHS

SEQ ID NO: 183 - protein, 7 aa, Artificial Sequence
  Synthetic sequence
ISRDGDS

SEQ ID NO: 184 - protein, 11 aa, Artificial Sequence
  Synthetic sequence
NARTSPWSAAL

SEQ ID NO: 185 - protein, 5 aa, Artificial Sequence
  Synthetic sequence
GSIFS

SEQ ID NO: 186 - protein, 8 aa, Artificial Sequence
  Synthetic sequence
IANGGTTT

SEQ ID NO: 187 - protein, 11 aa, Artificial Sequence
  Synthetic sequence
YAKRSWDSSSY

SEQ ID NO: 188 - protein, 8 aa, Artificial Sequence
  Synthetic sequence
GFTLDYYA

SEQ ID NO: 189 - protein, 8 aa, Artificial Sequence
  Synthetic sequence
INSGDSST

SEQ ID NO: 190 - protein, 21 aa, Artificial Sequence
  Synthetic sequence
ATVDTLIVAGNLEECRYDMDY

SEQ ID NO: 191 - protein, 217 aa, Artificial Sequence
  Synthetic sequence
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 15 aa, Artificial Sequence
  Synthetic sequence
EPKSADKTHTCPPCP

SEQ ID NO: 193 - protein, 125 aa, Artificial Sequence
  Synthetic sequence
EVQLVESGGGLVQPGGSLRLSCAASGSIFTSDDMDYAMGWYRQAPGKQREWVATITSDEDTYYVDSVKGRFTISRDNAKNSLYLQMNSLRAEDTAVYYCFANVRSDRSSLYRNYWGQGTMVTVSS

SEQ ID NO: 194 - protein, 125 aa, Artificial Sequence
  Synthetic sequence
EVQLVESGGGLVQPGGSLRLSCAASGSIFTSDDMDYAMGWYRQAPGKQREWVATITSDEDTNYVDSVKGRFTISRDNAKNSLYLQMNSLRAEDTAVYYCFANVRSDRSSLYRNYWGQGTMVTVSS

SEQ ID NO: 195 - protein, 125 aa, Artificial Sequence
  Synthetic sequence
EVQLVESGGGLVQPGGSLRLSCAASGSIFTSDDMDYAMGWYRQAPGKQREWVATITSDEDTNYVDSVKGRFTISRDNAKNSAYLQMNSLRAEDTAVYYCFANVRSDRSSLYRNYWGQGTMVTVSS

SEQ ID NO: 196 - protein, 125 aa, Artificial Sequence
  Synthetic sequence
QVQLVESGGGLVQPGGSLRLSCAASGSIFTSDDMDYAMGWYRQAPGKQREWVATITSDEDTNYVDSVKGRFTISRDNAKNSAYLQMNSLRAEDTAVYYCFANVRSDRSSLYRNYWGQGTMVTVSS

SEQ ID NO: 197 - protein, 125 aa, Artificial Sequence
  Synthetic sequence
EVQLVESGGGLVQPGGSLRLSCAASGSIFTSQDMDYAMGWYRQAPGKQREWVATITSDEDTNYVDSVKGRFTISRDNAKNSAYLQMNSLRAEDTAVYYCFANVRSDRSSLYRNYWGQGTMVTVSS

SEQ ID NO: 198 - protein, 125 aa, Artificial Sequence
  Synthetic sequence
EVQLVESGGGLVQPGGSLRLSCAASGSIFTSDDMDYAMGWYRQAPGKQREWVATITSDEDTNYGDSVKGRFTISRDNAKNSAYLQMNSLRAEDTAVYYCFANVRSDRSSLYRNYWGQGTMVTVSS

SEQ ID NO: 199 - protein, 125 aa, Artificial Sequence
  Synthetic sequence
EVQLVESGGGLVQPGGSLRLSCAASGSIFTSDDMDYAMGWYRQAPGKQREWVATITSDEDTNYVDSVKGRFTISRDSAKNSAYLQMNSLRAEDTAVYYCFANVRSDRSSLYRNYWGQGTMVTVSS

SEQ ID NO: 200 - protein, 125 aa, Artificial Sequence
  Synthetic sequence
EVQLVESGGGLVQPGGSLRLSCAASGSIFTSNDMDYAMGWYRQAPGKQREWVATITSDEDTNYVDSVKGRFTISRDSAKNSAYLQMNSLRAEDTAVYYCFANVRSDRSSLYRNYWGQGTMVTVSS

SEQ ID NO: 201 - protein, 125 aa, Artificial Sequence
  Synthetic sequence
EVQLVESGGGLVQPGGSLRLSCAASGSIFTSDDMDYAMGWYRQAPGKQREWVATITSDEDTNYVDSVKGRFTISRDNAKNSAYLQMNSLKPEDTAVYYCFANVRSDRSSLYRNYWGQGTMVTVSS

SEQ ID NO: 202 - protein, 130 aa, Artificial Sequence
  Synthetic sequence
EVQLVESGGGLVQPGGSLRLSCAASGIILDYYPIGWFRQAPGKEREGVAFITNSDDSTIYTNYADSVKGRFTISRDKNSLYLQMNSLRAEDTAVYYCAARSTYIVGRNENALNSADYDYWGQGTMVTVSS

SEQ ID NO: 203 - protein, 130 aa, Artificial Sequence
  Synthetic sequence
EVQLVESGGGLVQPGGSLRLSCAASGIILDYYPIGWFRQAPGKEREGVAFITNSDSSTIYTNYADSVKGRFTISRDKNSLYLQMNSLRAEDTAVYYCAARSTYIVGRNENALNSADYDYWGQGTMVTVSS

SEQ ID NO: 204 - protein, 130 aa, Artificial Sequence
  Synthetic sequence
EVQLVESGGGLVQPGGSLRLSCTAPGIILDYYPIGWFRQAPGKEREGVAFITNSDDSTIYTNYADSVKGRFTISRDKNSLYLQMNSLRAEDTAVYYCAARSTYIVGRNENALNSADYDYWGQGTMVTVSS

SEQ ID NO: 205 - protein, 130 aa, Artificial Sequence
  Synthetic sequence
EVQLVESGGGLVQPGGSLRLSCTAPGIILDYYPIGWFRQAPGKEREGVAFITNSDSSTIYTNYADSVKGRFTISRDKNSLYLQMNSLRAEDTAVYYCAARSTYIVGRNENALNSADYDYWGQGTMVTVSS

SEQ ID NO: 206 - protein, 130 aa, Artificial Sequence
  Synthetic sequence
EVQLVESGGGLVQPGGSLRLSCAASGIILDYYPIGWFRQAPGKEREGVAFITNSDDSTIYTNYADSVKGRFTISREKNTLYLQMNSLRAEDTAVYYCAARSTYIVGRNENALNSADYDYWGQGTMVTVSS

SEQ ID NO: 207 - protein, 130 aa, Artificial Sequence
  Synthetic sequence
EVQLVESGGGLVQPGGSLRLSCAASGIILDYYPIGWFRQAPGKEREGVAFITNSDSSTIYTNYADSVKGRFTISREKNTLYLQMNSLRAEDTAVYYCAARSTYIVGRNENALNSADYDYWGQGTMVTVSS

SEQ ID NO: 208 - protein, 130 aa, Artificial Sequence
  Synthetic sequence
EVQLVESGGGLVQPGGSLRLSCAASGIILDYYPIGWFRQAPGKGREGVAFITNSDDSTIYTNYADSVKGRFTISRDKNSLYLQMNSLRAEDTAVYYCAARSTYIVGRNENALNSADYDYWGQGTMVTVSS

SEQ ID NO: 209 - protein, 121 aa, Artificial Sequence
  Synthetic sequence
EVQLVESGGGLVQPGGSLRLSCAASRSTSSLSLMSWHRQAPGKQRELVASITSGGKANYVDSVKGRFTISRDNAKNSLYLQMNSLRAEDTAVYYCHAYDRAGTASGFGAAWGQGTMVTVSS

SEQ ID NO: 210 - protein, 121 aa, Artificial Sequence
  Synthetic sequence
EVQLVESGGGLVQPGGSLRLSCAASRSTSSLSLMSWHRQAPGKQRELVASITSGGKANYVDSVKGRFTMSRDNAKNSVYLQMNSLRAEDTAVYYCHAYDRAGTASGFGAAWGQGTMVTVSS

SEQ ID NO: 211 - protein, 121 aa, Artificial Sequence
  Synthetic sequence
EVQLVESGGGLVQPGGSLRLSCAASRSTSSLSLMSWHRQAPGKQRELVASITSGGKANYVDSVKGRFTISRDDAKNSLYLQMNSLRAEDTAVYYCHAYDRAGTASGFGAAWGQGTMVTVSS

SEQ ID NO: 212 - protein, 121 aa, Artificial Sequence
  Synthetic sequence
EVQLVESGGGLVQPGGSLRLSCAASRSTSSLSLMSWHRQAPGKQRELVASITSGGKANYVDSVKGRFTISRNDAKNSLYLQMNSLRAEDTAVYYCHAYDRAGTASGFGAAWGQGTMVTVSS

SEQ ID NO: 213 - protein, 121 aa, Artificial Sequence
  Synthetic sequence
EVQLVESGGGLVQPGGSLRLSCAASRSTSSLSLMSWHRQAPGKQRELVASITSGGKANYVDSVKGRFTISRDNAKNSLYLQMNSLKPEDTAVYYCHAYDRAGTASGFGAAWGQGTMVTVSS

SEQ ID NO: 214 - protein, 120 aa, Artificial Sequence
  Synthetic sequence
EVQLVESGGGLVQPGGSLRLSCAASGFTFSINAAWYRQAPGKQRELVASISYGGSTNYADRVAGRFTISRDNAKNSLYLQMNSLRAEDTAVYYCRLDPGLALNNRPLYYWGQGTMVTVSS

SEQ ID NO: 215 - protein, 120 aa, Artificial Sequence
  Synthetic sequence
EVQLVESGGGLVQPGGSLRLSCAASGGTFVINAAWYRQAPGKQRELVASISYGGSTNYADRVAGRFTISRDNAKNSLYLQMNSLRAEDTAVYYCRLDPGLALNNRPLYYWGQGTMVTVSS

SEQ ID NO: 216 - protein, 120 aa, Artificial Sequence
  Synthetic sequence
QVQLVESGGGLVQPGGSLRLSCAASGGTFVINAAWYRQAPGKQRELVASISYGGSTNYADRVAGRFTISRDNAKNSLYLQMNSLRAEDTAVYYCRLDPGLALNNRPLYYWGQGTMVTVSS

SEQ ID NO: 217 - protein, 120 aa, Artificial Sequence
  Synthetic sequence
EVQLVESGGGLVQPGGSLRLSCAASGGTFVINAAWYRQAPGKQRELVASISYGGSTNYADRVAGRFTISRDNAKNSVYLQMNSLRAEDTAVYYCRLDPGLALNNRPLYYWGQGTMVTVSS

SEQ ID NO: 218 - protein, 117 aa, Artificial Sequence
  Synthetic sequence
EVQLVESGGGLVQPGGSLRLSCAASGFTFSLHSMGWYRQAPGKQRELVAGISRDGDSNYLSSLAGRFTISRDNAKNSLYLQMNSLRAEDTAVYYCNARTSPWSAALWGQGTMVTVSS

SEQ ID NO: 219 - protein, 117 aa, Artificial Sequence
  Synthetic sequence
EVQLVESGGGLVQPGGSLRLSCAASGSTFSLHSMGWYRQAPGKQRELVAGISRDGDSNYLSSLAGRFTISRDNAKNSLYLQMNSLRAEDTAVYYCNARTSPWSAALWGQGTMVTVSS

SEQ ID NO: 220 - protein, 117 aa, Artificial Sequence
  Synthetic sequence
EVQLVESGGGLVQPGGSLRLSCAASGSTLSLHSMGWYRQAPGKQRELVAGISRDGDSNYLSSLAGRFTISRDNAKNSLYLQMNSLRAEDTAVYYCNARTSPWSAALWGQGTMVTVSS

SEQ ID NO: 221 - protein, 117 aa, Artificial Sequence
  Synthetic sequence
EVQLVESGGGLVQPGGSLRLSCAASGSTLSLHSMGWYRQAPGKQRELVAGISRDGDSNYLSSLAGRFTISRDGTKNSLYLQMNSLRAEDTAVYYCNARTSPWSAALWGQGTMVTVSS

SEQ ID NO: 222 - protein, 117 aa, Artificial Sequence
  Synthetic sequence
EVQLVESGGGLVQPGGSLRLSCAASGSTLSLHSMGWYRQAPGKQRELVAGISRDGDSNYLSSLAGRFTISRDGTKNSVYLQMNSLRAEDTAVYYCNARTSPWSAALWGQGTMVTVSS

SEQ ID NO: 223 - protein, 118 aa, Artificial Sequence
  Synthetic sequence
EVQVVESGGGLVQPGGSLRLSCAASGFTFSNYAMSWVRQAPGKGLEWVAEINSGGDNTYYAYFVKGRFTISRDNAKNSLYLQMNSLKPEDTATYYCAREEENAGTILQGQGTQVTVSS

SEQ ID NO: 224 - protein, 98 aa, Artificial Sequence
  Synthetic sequence
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAR

SEQ ID NO: 225 - protein, 11 aa, Artificial Sequence
  Synthetic sequence
WGQGTMVTVSS

SEQ ID NO: 226 - protein, 12 aa, Artificial Sequence
  Synthetic sequence
GSIFTSQDMDYA

SEQ ID NO: 227 - protein, 12 aa, Artificial Sequence
  Synthetic sequence
GSIFTSNDMDYA

SEQ ID NO: 228 - protein, 20 aa, Artificial Sequence
  Synthetic sequence
RSTYIVGRNENALNSADYDY

SEQ ID NO: 229 - protein, 20 aa, Artificial Sequence
  Synthetic sequence
FITNSDSSTIYTNYADSVKG

SEQ ID NO: 230 - protein, 16 aa, Artificial Sequence
  Synthetic sequence
SISYGGSTNYADRVAG

SEQ ID NO: 231 - protein, 5 aa, Artificial Sequence
  Synthetic sequence
GGGGS